(19) United States
(12) Patent Application Publication
(10) Pub. No.: US 2022/0064665 A1
(43) Pub. Date: Mar. 3, 2022

(54) METHODS OF GENETICALLY ALTERING A PLANT NIN-GENE TO BE RESPONSIVE TO CYTOKININ

(71) Applicant: Wageningen Universiteit, Wageningen (NL)

(72) Inventors: Rene GEURTS, Wageningen (NL); Jieyu LIU, Wageningen (NL); Luuk RUTTEN, Wageningen (NL); Olga KULIKOVA, Wageningen (NL); Ton BISSELING, Wageningen (NL)

(73) Assignee: Wageningen Universiteit, Wageningen (NL)

(21) Appl. No.: 17/298,916

(22) PCT Filed: Dec. 5, 2019
(86) PCT No.: PCT/EP2019/083770

§ 371 Date: Jun. 1, 2021

Related U.S. Application Data
(60) Provisional application No. 62/776,325, filed on Dec. 6, 2018.

(51) Int. Cl. C12N 15/82

(52) U.S. Cl. CPC C12N 15/8295; C12N 15/8227

(57) ABSTRACT

Aspects of the present disclosure relate to genetically modified plants comprising NODULE INCEPTION (NIN) and NIN-LIKE PROTEIN (NLP) that have been genetically altered to be responsive to cytokinin so that the NIN or NLP protein can induce root nodulation upon appropriate signaling.

### CROSS-REFERENCE TO RELATED APPLICATION

This application claims the benefit of U.S. Provisional Application No. 62/776,325, filed Dec. 6, 2018, which is hereby incorporated by reference in its entirety.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: 794542000540SEQLIST.TXT, date recorded: Nov. 25, 2019, size: 3,802 KB).

### TECHNICAL FIELD

The present disclosure relates to genetically altered plants. In particular, the present disclosure relates to plants with NODULE INCEPTION (NIN) and NIN-LIKE PROTEIN (NLP) genes that have been genetically altered to be responsive to cytokinin so that the NIN or NLP protein can induce root nodulation upon appropriate signaling.

### BACKGROUND

Nodulating plant species, such as legumes, Parasponia spp., and actinorhizal plants, have evolved to form symbiotic relationships with nitrogen fixing bacteria. They form specialized organs called nodules to house these bacteria, which provide an optimal environment for the bacteria to fix nitrogen and provide it to the plant. In turn, the plant provides the bacteria with carbohydrates and other resources. The initial step of nodule formation is the recognition of the presence of symbiotic bacteria, for example by the detection of lipo-chitooligosaccharides (also known as Nod factors in the case of rhizobial bacteria) produced by the bacteria. Recognition of such symbiotic signals induces nodule organogenesis and allows bacterial infection.

Genetic screens have identified genes involved in the nodule organogenesis process. Chief among these is the transcription factor NODULE INCEPTION (NIN), which has been shown to have a key role in nodule organogenesis across multiple plant species, such as legumes and Casuarina glauca (Clavijo et al., New Phytol. 208: 887-903 (2015)). While the essential role of NIN has long been known, attempts to complement nin mutant plants have consistently failed. This is attested to by numerous examples in the literature. Clavijo et al. used a 2175 bp Medicago truncatula NIN promoter, and reported, “unfortunately it was not possible to complement M. truncatula nin mutants for infection even with the ProMtNIN:MtNIN construct” (Clavijo et al., New Phytol. 208: 887-903 (2015); quote from pg. 898). In characterizing the Lotus japonicus daphne mutant, Yoro et al. stated, “although NIN is a key transcription factor in nodule development, the functional NIN promoter region necessary for nodule organogenesis has not yet been elucidated” and observed “only IT formation, and not nodule formation, was rescued in the L. japonicus nin-9 mutant by the introduction of the L. japonicus based construct ProNIN(˜4 kb)::NIN::TerNIN” (Yoro et al., Plant Physiol. 165:747-758 (2014); quotes from pg. 756). Finally, Vernie et al. found that expression of M. truncatula NIN using a 2.18 kb MtNIN promoter was only partially able to complement a M. truncatula nin null mutant, which when transformed produced a very low number of apparently non-functional nodules a long time (fifty days) after inoculation (Vernie et al., The Plant Cell, 27:3410-3424 (2015)).

This inability to complement legume nin mutant plants has meant that the precise mechanism of NIN involvement in organogenesis has remained elusive, because important components of NIN regulation and integration with nodule organogenesis processes were not known. More importantly, as NIN is the key player in nodule organogenesis, a successful nodule engineering approach needs to incorporate NIN. The failures to complement nin mutants over the past 20 years have been a huge roadblock to developing nodule engineering strategies. In order to successfully engineer nodulation, the ability to complement nin mutants, and the knowledge of NIN regulation that comes with that ability, will be required. There exists a clear need for identifying NIN regulatory regions that in combination with the NIN coding sequence will allow full complementation of nin mutants.

### BRIEF SUMMARY

In order to meet these needs, the present disclosure provides means of fully complementing legume nin mutants by introduction of cytokinin-responsive elements into a regulatory region operably linked with the NIN coding sequence. The present disclosure further provides means of introducing cytokinin-responsive elements into plants operably linked with a NIN or NLP coding sequence that may be endogenous or heterologous.

An aspect of the disclosure includes a genetically altered plant, wherein the plant or a part thereof includes one or more genetic alterations that increase activity of a NODULE INCEPTION (NIN) protein or a NIN-like protein (NLP protein) in response to cytokinin signaling as compared to a wild type (WT) plant without the one or more genetic alterations, and wherein the plant or the part thereof includes a nucleic acid encoding the NIN protein or the NLP protein. An additional embodiment of this aspect includes the one or more genetic alterations being addition of one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, or twenty-four or more cytokinin response elements operably linked to the nucleic acid encoding the NIN protein or the NLP protein. Yet another embodiment of this aspect includes at least one of the cytokinin response elements being a B-type cytokinin signaling RESPONSE REGULATOR (RR) binding site. A further embodiment of this aspect includes at least one of the B-type cytokinin signaling RR binding sites having the sequence of SEQ ID NO:613 or SEQ ID NO:614. Still another embodiment of this aspect includes at least one of the B-type cytokinin signaling RR binding sites having the sequence selected from the group of SEQ ID NO:551, SEQ ID NO:552, SEQ ID NO:553, SEQ ID NO:554, SEQ ID NO:555, SEQ ID NO:556, SEQ ID NO:557, SEQ ID NO:558, SEQ ID NO:559, SEQ ID NO:560, SEQ ID NO:561, SEQ ID NO:562, SEQ ID NO:563, SEQ ID NO:564, SEQ ID NO:565, SEQ ID NO:566, SEQ ID NO:567, SEQ ID NO:568, SEQ ID NO:569, SEQ ID NO:570, SEQ ID NO:571, SEQ ID NO:572, SEQ ID NO:573, SEQ ID NO:574, SEQ ID NO:575, SEQ ID NO:576, SEQ ID NO:577, SEQ ID NO:578, SEQ ID NO:579, SEQ ID NO:580, SEQ ID NO:581, SEQ ID NO:582, SEQ ID NO:583, SEQ ID NO:584, SEQ ID NO:585, SEQ ID NO:586, SEQ ID NO:587, SEQ ID NO:588, SEQ ID NO:589, SEQ ID NO:590, SEQ ID NO:591, SEQ ID NO:592, SEQ ID NO:593, SEQ ID NO:594, SEQ ID NO:595, SEQ ID NO:596, SEQ ID NO:597, SEQ ID NO:598, SEQ ID NO:599, SEQ ID NO:600, SEQ ID NO:601, SEQ ID NO:602, SEQ ID NO:603, SEQ ID NO:604, SEQ ID NO:605, SEQ ID NO:606, SEQ ID NO:607, SEQ ID NO:608, SEQ ID NO:609, SEQ ID NO:610, SEQ ID NO:611, SEQ ID NO:612, SEQ ID NO:615, SEQ ID NO:616, SEQ ID NO:617, SEQ ID NO:618, SEQ ID NO:619, SEQ ID NO:620, SEQ ID NO:621, SEQ ID NO:622, SEQ ID NO:623, SEQ ID NO:624, SEQ ID NO:625, or SEQ ID NO:626.

In yet another embodiment, which may be combined with any of the preceding embodiments, the cytokinin response elements are within 100 nucleotides, within 90 nucleotides, within 86 nucleotides, within 80 nucleotides, within 70 nucleotides, within 60 nucleotides, within 50 nucleotides, within 40 nucleotides, within 30 nucleotides, within 25 nucleotides, within 20 nucleotides, within 19 nucleotides, within 18 nucleotides, within 17 nucleotides, within 16 nucleotides, within 15 nucleotides, within 14 nucleotides, within 13 nucleotides, within 12 nucleotides, within 11 nucleotides, within 10 nucleotides, within 9 nucleotides, within 8 nucleotides, within 7 nucleotides, or within 6 nucleotides of each other. In an additional embodiment of this aspect, the cytokinin response elements are within 11 nucleotides of each other. In yet another embodiment, which may be combined with any of the preceding embodiments, the nucleic acid encoding the NIN protein or the NLP protein is operably linked to a promoter that is operably linked to the cytokinin response elements. In an additional embodiment of this aspect, the promoter and the cytokinin response elements are within 60,000 nucleotides, within 55,000 nucleotides, within 50,000 nucleotides, within 45,000 nucleotides, within 42,000 nucleotides, within 40,000 nucleotides, within 35,000 nucleotides, within 30,000 nucleotides, within 25,000 nucleotides, within 20,000 nucleotides, within 15,000 nucleotides, within 10,000 nucleotides, within 9,000 nucleotides, within 8,000 nucleotides, within 7,000 nucleotides, within 6,000 nucleotides, within 5,000 nucleotides, within 4,000 nucleotides, within 3,000 nucleotides, within 2,000 nucleotides, within 1,000 nucleotides, within 500 nucleotides, within 400 nucleotides, within 300 nucleotides, within 200 nucleotides, or within 100 nucleotides of each other.

Still another embodiment of this aspect that can be combined with any of the preceding embodiments includes the nucleic acid encoding a NIN/NLP1 orthogroup protein. An additional embodiment of this aspect includes the NIN/NLP1 orthogroup protein having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group of SEQ ID NO:1, SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22; SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:108, SEQ ID NO:109, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, and SEQ ID NO:236. A further embodiment of this aspect includes the NIN/NLP1 orthogroup protein being a NIN protein and having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO:22 (i.e., CsaNIN; Cannabis sativa), SEQ ID NO:78 (i.e., HluNIN; Humulus lupulus), SEQ ID NO:89 (i.e., LjNIN; Lotus japonicus), SEQ ID NO:108 (i.e., MtNIN; Medicago truncatula); SEQ ID NO:136 (i.e., PanNIN; Parasponia andersonii), SEQ ID NO:139 (i.e., PriNIN; Parasponia rigida), SEQ ID NO:142 (i.e., PruNIN; Parasponia rugosa), SEQ ID NO:185 (i.e., TleNIN; Trema levigata), SEQ ID NO:187 (i.e., TorNIN; Trema orientalis), SEQ ID NO:190 (i.e., TtoNIN; Trema tomentosa), and SEQ ID NO:236 (i.e., ZjuNIN; Ziziphus jujuba). Still another embodiment of this aspect includes the NIN/NLP1 orthogroup protein being a NIN protein and having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group of SEQ ID NO:89 (i.e., LjNIN; Lotus japonicus) or SEQ ID NO:108 (i.e., MtNIN; Medicago truncatula).

Yet another embodiment of this aspect that can be combined with any of the preceding embodiments that has a genetically altered plant includes the nucleic acid encoding a NLP2-3 orthogroup protein, a NLP4 orthogroup protein, or a basal NIN/NLP orthogroup protein. An additional embodiment of this aspect includes the NLP2-3 orthogroup protein having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group of SEQ ID NO:237, SEQ ID NO:238, SEQ ID NO:239, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, SEQ ID NO:282, SEQ ID NO:283, SEQ ID NO:284, SEQ ID NO:285, SEQ ID NO:286, SEQ ID NO:287, SEQ ID NO:288, SEQ ID NO:289, SEQ ID NO:290, SEQ ID NO:291, SEQ ID NO:292, SEQ ID NO:293, SEQ ID NO:294, SEQ ID NO:295, SEQ ID NO:296, SEQ ID NO:297, SEQ ID NO:298, SEQ ID NO:299, SEQ ID NO:300, SEQ ID NO:301, SEQ ID NO:302, SEQ ID NO:303, SEQ ID NO:304, SEQ ID NO:305, SEQ ID NO:306, SEQ ID NO:307, SEQ ID NO:308, SEQ ID NO:309, SEQ ID NO:310, SEQ ID NO:311, SEQ ID NO:312, SEQ ID NO:313, SEQ ID NO:314, SEQ ID NO:315, SEQ ID NO:316, SEQ ID NO:317, SEQ ID NO:318, SEQ ID NO:319, SEQ ID NO:320, SEQ ID NO:321, SEQ ID NO:322, SEQ ID NO:323, SEQ ID NO:324, SEQ ID NO:325, SEQ ID NO:326, SEQ ID NO:327, SEQ ID NO:328, SEQ ID NO:329, SEQ ID NO:332, SEQ ID NO:333, SEQ ID NO:334, SEQ ID NO:335, SEQ ID NO:336, SEQ ID NO:337, SEQ ID NO:338, SEQ ID NO:339, SEQ ID NO:340, SEQ ID NO:341, SEQ ID NO:342, SEQ ID NO:343, SEQ ID NO:344, SEQ ID NO:345, SEQ ID NO:346, SEQ ID NO:347, SEQ ID NO:348, SEQ ID NO:349, SEQ ID NO:350, SEQ ID NO:351, SEQ ID NO:352, SEQ ID NO:353, SEQ ID NO:354, SEQ ID NO:355, SEQ ID NO:356, SEQ ID NO:357, SEQ ID NO:358, SEQ ID NO:359, SEQ ID NO:360, SEQ ID NO:361, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:371, SEQ ID NO:372, SEQ ID NO:373, SEQ ID NO:374, SEQ ID NO:375, SEQ ID NO:376, and SEQ ID NO:377. Yet another embodiment of this aspect includes the NLP4 orthogroup protein having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group of SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:391, SEQ ID NO:392, SEQ ID NO:393, SEQ ID NO:394, SEQ ID NO:395, SEQ ID NO:396, SEQ ID NO:397, SEQ ID NO:398, SEQ ID NO:399, SEQ ID NO:400, SEQ ID NO:401, SEQ ID NO:402, SEQ ID NO:403, SEQ ID NO:404, SEQ ID NO:405, SEQ ID NO:406, SEQ ID NO:408, SEQ ID NO:409, SEQ ID NO:410, SEQ ID NO:411, SEQ ID NO:412, SEQ ID NO:413, SEQ ID NO:414, SEQ ID NO:415, SEQ ID NO:417, SEQ ID NO:418, SEQ ID NO:419, SEQ ID NO:420, SEQ ID NO:421, SEQ ID NO:422, SEQ ID NO:423, SEQ ID NO:424, SEQ ID NO:425, SEQ ID NO:426, SEQ ID NO:427, SEQ ID NO:428, SEQ ID NO:429, SEQ ID NO:430, SEQ ID NO:431, SEQ ID NO:432, SEQ ID NO:433, SEQ ID NO:434, SEQ ID NO:435, SEQ ID NO:436, SEQ ID NO:437, SEQ ID NO:438, SEQ ID NO:439, SEQ ID NO:440, SEQ ID NO:441, SEQ ID NO:442, SEQ ID NO:443, SEQ ID NO:444, SEQ ID NO:445, SEQ ID NO:446, SEQ ID NO:447, SEQ ID NO:448, SEQ ID NO:449, SEQ ID NO:450, SEQ ID NO:451, SEQ ID NO:452, SEQ ID NO:453, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, SEQ ID NO:484, SEQ ID NO:485, SEQ ID NO:486, SEQ ID NO:487, SEQ ID NO:488, SEQ ID NO:489, SEQ ID NO:490, SEQ ID NO:491, SEQ ID NO:492, SEQ ID NO:493, SEQ ID NO:494, SEQ ID NO:495, SEQ ID NO:496, SEQ ID NO:497, SEQ ID NO:498, SEQ ID NO:499, SEQ ID NO:500, SEQ ID NO:501, SEQ ID NO:502, SEQ ID NO:504, SEQ ID NO:505, SEQ ID NO:506, SEQ ID NO:507, SEQ ID NO:508, SEQ ID NO:509, SEQ ID NO:510, SEQ ID NO:511, SEQ ID NO:512, SEQ ID NO:513, SEQ ID NO:514, SEQ ID NO:515, SEQ ID NO:516, SEQ ID NO:517, SEQ ID NO:518, SEQ ID NO:519, SEQ ID NO:520, SEQ ID NO:521, SEQ ID NO:522, SEQ ID NO:523, and SEQ ID NO: 524. A further embodiment of this aspect includes the basal NIN/NLP orthogroup protein having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group of SEQ ID NO:637, SEQ ID NO:638, SEQ ID NO:639, SEQ ID NO:640, SEQ ID NO:641, SEQ ID NO:642, SEQ ID NO:643, SEQ ID NO:644, SEQ ID NO:645, SEQ ID NO:646, SEQ ID NO:647, SEQ ID NO:648, SEQ ID NO:649, SEQ ID NO:650, SEQ ID NO:651, SEQ ID NO:652, SEQ ID NO:653, SEQ ID NO:654, SEQ ID NO:655, SEQ ID NO:656, SEQ ID NO:657, SEQ ID NO:658, SEQ ID NO:659, SEQ ID NO:660, SEQ ID NO:661, or SEQ ID NO:662.

In yet another embodiment, which may be combined with any of the preceding embodiments, the nucleic acid encoding the NIN protein or the NLP protein is endogenous. Still another embodiment of this aspect that can be combined with any of the preceding embodiments includes the nucleic acid encoding the NIN protein or the NLP protein being heterologous. Yet another embodiment of this present aspect that can be combined with any of the preceding aspects that has the nucleic acid encoding the NIN protein or the NLP protein operably linked to a promoter includes the promoter being endogenous. Still another embodiment of this aspect that can be combined with any of the preceding aspects that has the nucleic acid encoding the NIN protein or the NLP protein operably linked to a promoter includes the promoter being heterologous.

In still another embodiment, which may be combined with any of the preceding embodiments, cytokinin signaling or induction of the cytokinin signaling pathway in a root pericycle cell layer, a root endodermis cell layer (i.e., endodermal cell layer), root cortex cell layers (i.e., cortical cell layer), and/or a root epidermis cell layer (i.e., epidermal cell layer) induces nodule organogenesis. Still another embodiment of this aspect that can be combined with any of the preceding embodiments further includes one or more CYCLOPS response elements operably linked to the nucleic acid. An additional embodiment of this aspect includes CYCLOPS expression in a root epidermis cell layer (i.e., epidermal cell layer) inducing rhizobium infection.

In a further embodiment, which may be combined with any of the preceding embodiments, the genetically altered plant is a monocot. An additional embodiment of this aspect includes the genetically altered plant being selected from the group of corn, rice, wheat, barley, sorghum, millet, oat, or rye. Still another embodiment of this aspect that can be combined with any of the preceding embodiments further includes the genetically altered plant being selected from the group of apple, pear, plum, apricot, peach, almond, walnut, cherry, strawberry, raspberry, blackberry, red currant, black currant, melon, cucumber, pumpkin, squash, grape, hemp, hops, birch, beech, jujube, cassava, poplar, chestnut, citrus, potato, tomato, sweet potato, Trema spp., and Jatropha spp. In yet another embodiment, which may be combined with any of the preceding embodiments, the WT plant is not a legume, does not form nodules for symbiosis with nitrogen fixing bacteria, or both is not a legume and does not form nodules for symbiosis with nitrogen fixing bacteria.

Still another embodiment of this aspect that can be combined with any of the preceding embodiments includes a genetically altered plant part of the genetically altered plant of any one of the preceding embodiments with respect to plant parts, wherein the plant part is a leaf, a stem, a root, a tuber, a flower, a seed, a kernel, a grain, a fruit, a cell, or a portion thereof and the genetically altered plant part includes the one or more genetic alterations. An additional embodiment of this aspect includes the plant part being a fruit, a tuber, a kernel, or a grain. Yet another embodiment of this aspect that can be combined with any of the preceding embodiments with respect to pollen grain or ovules includes a genetically altered pollen grain or a genetically altered ovule of the plant of any one of the preceding embodiments, wherein the genetically altered pollen grain or the genetically altered ovule includes the one or more genetic alterations. A further embodiment of this aspect that can be combined with any of the preceding embodiments includes a genetically altered protoplast produced from the genetically altered plant of any of the preceding embodiments, wherein the genetically altered protoplast includes the one or more genetic alterations. An additional embodiment of this aspect that can be combined with any of the preceding embodiments includes a genetically altered tissue culture produced from protoplasts or cells from the genetically altered plant of any one of the preceding embodiments, wherein the cells or protoplasts are produced from a plant part selected from the group of leaf, leaf mesophyll cell, anther, pistil, stem, petiole, root, root tip, tuber, fruit, seed, kernel, grain, flower, cotyledon, hypocotyl, embryo, or meristematic cell, wherein the genetically altered tissue culture includes the one or more genetic alterations. An additional embodiment of this aspect includes a genetically altered plant regenerated from the genetically altered tissue culture that includes the one or more genetic alterations. Still another embodiment of this aspect that can be combined with any of the preceding embodiments that has a genetically altered plant includes the genetically altered plant having all the physiological and morphological characteristics of the plant of any of the preceding embodiments. Yet another embodiment of this aspect that can be combined with any of the preceding embodiments that has a genetically altered plant includes a genetically altered plant seed produced from the genetically altered plant of any one of the preceding embodiments. A further embodiment of this aspect that can be combined with any of the preceding embodiments that has a genetically altered plant includes the seed of the plant producing a plant having all the physiological and morphological characteristics of the plant of any of the above embodiments.

An additional aspect of the disclosure includes methods of producing the genetically altered plant of any of the preceding embodiments that has a genetically altered plant, including the steps of: (a) introducing the one or more genetic alterations into a plant cell, tissue, or other explant; (b) regenerating the plant cell, tissue, or other explant into a genetically altered plantlet; and (c) growing the genetically altered plantlet into a genetically altered plant with the one or more genetic alterations that increase activity of the NIN protein or the NLP protein in response to cytokinin signaling as compared to an untransformed WT plant. An additional embodiment of this aspect further includes identifying successful introduction of the one or more genetic alterations by screening or selecting the plant cell, tissue, or other explant prior to step (b); screening or selecting plantlets between step (b) and (c); or screening or selecting plants after step (c). In yet another embodiment, which may be combined with any of the preceding embodiments, transformation is done using a transformation method selected from the group of particle bombardment (i.e., biolistics, gene gun), Agrobacterium-mediated transformation, Rhizobium-mediated transformation, or protoplast transfection or transformation.

Still another embodiment of this aspect that can be combined with any of the preceding embodiments includes genetic alterations being introduced with a vector. An additional embodiment of this aspect includes the vector including a promoter operably linked to a nucleotide encoding a NIN or NLP protein and one or more cytokinin response elements operably linked to the promoter. Yet another embodiment of this aspect includes the promoter and the one or more cytokinin response elements being selected from the group of a NIN gene promoter comprising a 5′-upstream sequence comprising a CYCLOPS-binding box through a transcription start site of the NIN gene operably linked to a 3C region, the NIN gene promoter comprising a 5′-upstream sequence comprising the CYCLOPS-binding box through to the transcription start site of the NIN gene operably linked to a CE region, a minimal promoter operably linked to a CYCLOPS-binding box operably linked to a CE region, and a minimal promoter operably linked to a CYCLOPS-binding box operably linked to one or more cytokinin response elements. In a further embodiment of this aspect, the vector includes one or more gene editing components that target a nuclear genome sequence operably linked to an endogenous NIN protein or NLP protein. Yet another embodiment of this aspect includes the nuclear genome sequence being edited by the one or more gene editing components to introduce a cis-regulatory element selected from the group of one or more cytokinin response elements, a 3C region, or a CE region. Yet another embodiment of this present aspect that can be combined with any of the preceding aspects that has a vector including one or more gene editing components includes one or more gene editing components being selected from the group of a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (ODN), wherein the ODN targets the nuclear genome sequence; or a vector including a CRISPR/Cas enzyme encoding sequence and a targeting sequence, wherein the targeting sequence targets the nuclear genome sequence.

A further embodiment of this present aspect that can be combined with any of the preceding aspects that has genetic alterations being introduced with a vector includes the NIN gene promoter, 3C region, CE region, CYCLOPS-binding box, or one or more cytokinin response elements being from a nodulating legume species. An additional embodiment of this aspect includes the nodulating legume species being selected from the group of peanut, pigeon pea, chickpea, soybean, velvet bean, bean, pea, adzuki bean, mung bean, clover, lupine, Lotus japonicus, and Medicago truncatula. Yet another embodiment of this present aspect that can be combined with any of the preceding aspects that has genetic alterations being introduced with a vector wherein the vector includes the NIN gene promoter, 3C region, CE region, CYCLOPS-binding box, or one or more cytokinin response elements from a nodulating legume species includes cytokinin response elements being selected from the group of SEQ ID NO:551, SEQ ID NO:552, SEQ ID NO:553, SEQ ID NO:554, SEQ ID NO:555, SEQ ID NO:556, SEQ ID NO:557, SEQ ID NO:558, SEQ ID NO:559, SEQ ID NO:560, SEQ ID NO:561, SEQ ID NO:562, SEQ ID NO:563, SEQ ID NO:564, SEQ ID NO:565, SEQ ID NO:566, SEQ ID NO:567, SEQ ID NO:568, SEQ ID NO:569, SEQ ID NO:570, SEQ ID NO:571, SEQ ID NO:572, SEQ ID NO:573, SEQ ID NO:574, SEQ ID NO:575, SEQ ID NO:576, SEQ ID NO:577, SEQ ID NO:578, SEQ ID NO:579, SEQ ID NO:580, SEQ ID NO:581, SEQ ID NO:582, SEQ ID NO:583, SEQ ID NO:584, SEQ ID NO:585, SEQ ID NO:586, SEQ ID NO:587, SEQ ID NO:588, SEQ ID NO:589, SEQ ID NO:590, SEQ ID NO:591, SEQ ID NO:592, SEQ ID NO:593, SEQ ID NO:594, SEQ ID NO:595, SEQ ID NO:596, SEQ ID NO:597, SEQ ID NO:598, SEQ ID NO:599, SEQ ID NO:600, SEQ ID NO:601, SEQ ID NO:602, SEQ ID NO:603, SEQ ID NO:604, SEQ ID NO:605, SEQ ID NO:606, SEQ ID NO:607, SEQ ID NO:608, SEQ ID NO:609, SEQ ID NO:610, SEQ ID NO:611, or SEQ ID NO:612. Still another embodiment of this present aspect that can be combined with any of the preceding aspects that has genetic alterations being introduced with a vector includes cytokinin response elements being selected from the group of SEQ ID NO:613, SEQ ID NO:614, SEQ ID NO:615, SEQ ID NO:616, SEQ ID NO:617, SEQ ID NO:618, SEQ ID NO:619, SEQ ID NO:620, SEQ ID NO:621, SEQ ID NO:622, SEQ ID NO:623, SEQ ID NO:624, SEQ ID NO:625, and SEQ ID NO:626.

A further embodiment of this present aspect that can be combined with any of the preceding aspects that has genetic alterations being introduced with a vector includes the NIN or NLP protein being a NIN/NLP1 orthogroup protein and having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group of SEQ ID NO:1, SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22; SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:108, SEQ ID NO:109, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, and SEQ ID NO:236. Yet another embodiment of this present aspect that can be combined with any of the preceding aspects that has genetic alterations being introduced with a vector includes the NIN/NLP1 orthogroup protein being a NIN protein and having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group SEQ ID NO:22 (i.e., CsaNIN; Cannabis sativa), SEQ ID NO:78 (i.e., HluNIN; Humulus lupulus), SEQ ID NO:89 (i.e., LjNIN; Lotus japonicus), SEQ ID NO:108 (i.e., MtNIN; Medicago truncatula); SEQ ID NO:136 (i.e., PanNIN; Parasponia andersonii), SEQ ID NO:139 (i.e., PriNIN; Parasponia rigida), SEQ ID NO:142 (i.e., PruNIN; Parasponia rugosa), SEQ ID NO:185 (i.e., TleNIN; Trema levigata), SEQ ID NO:187 (i.e., TorNIN; Trema orientalis), SEQ ID NO:190 (i.e., TtoNIN; Trema tomentosa), and SEQ ID NO:236 (i.e., ZjuNIN; Ziziphus jujuba). A further embodiment of this present aspect that can be combined with any of the preceding aspects that has genetic alterations being introduced with a vector includes the NIN/NLP1 orthogroup protein being a NIN protein and having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group SEQ ID NO:89 (i.e., LjNIN; Lotus japonicus) or SEQ ID NO:108 (i.e., MtNIN; Medicago truncatula). Still another embodiment of this present aspect that can be combined with any of the preceding aspects that has genetic alterations being introduced with a vector includes the NIN or NLP protein being a NLP2-3 orthogroup protein and having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group of SEQ ID NO:237, SEQ ID NO:238, SEQ ID NO:239, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, SEQ ID NO:282, SEQ ID NO:283, SEQ ID NO:284, SEQ ID NO:285, SEQ ID NO:286, SEQ ID NO:287, SEQ ID NO:288, SEQ ID NO:289, SEQ ID NO:290, SEQ ID NO:291, SEQ ID NO:292, SEQ ID NO:293, SEQ ID NO:294, SEQ ID NO:295, SEQ ID NO:296, SEQ ID NO:297, SEQ ID NO:298, SEQ ID NO:299, SEQ ID NO:300, SEQ ID NO:301, SEQ ID NO:302, SEQ ID NO:303, SEQ ID NO:304, SEQ ID NO:305, SEQ ID NO:306, SEQ ID NO:307, SEQ ID NO:308, SEQ ID NO:309, SEQ ID NO:310, SEQ ID NO:311, SEQ ID NO:312, SEQ ID NO:313, SEQ ID NO:314, SEQ ID NO:315, SEQ ID NO:316, SEQ ID NO:317, SEQ ID NO:318, SEQ ID NO:319, SEQ ID NO:320, SEQ ID NO:321, SEQ ID NO:322, SEQ ID NO:323, SEQ ID NO:324, SEQ ID NO:325, SEQ ID NO:326, SEQ ID NO:327, SEQ ID NO:328, SEQ ID NO:329, SEQ ID NO:332, SEQ ID NO:333, SEQ ID NO:334, SEQ ID NO:335, SEQ ID NO:336, SEQ ID NO:337, SEQ ID NO:338, SEQ ID NO:339, SEQ ID NO:340, SEQ ID NO:341, SEQ ID NO:342, SEQ ID NO:343, SEQ ID NO:344, SEQ ID NO:345, SEQ ID NO:346, SEQ ID NO:347, SEQ ID NO:348, SEQ ID NO:349, SEQ ID NO:350, SEQ ID NO:351, SEQ ID NO:352, SEQ ID NO:353, SEQ ID NO:354, SEQ ID NO:355, SEQ ID NO:356, SEQ ID NO:357, SEQ ID NO:358, SEQ ID NO:359, SEQ ID NO:360, SEQ ID NO:361, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:371, SEQ ID NO:372, SEQ ID NO:373, SEQ ID NO:374, SEQ ID NO:375, SEQ ID NO:376, and SEQ ID NO:377. A further embodiment of this present aspect that can be combined with any of the preceding aspects that has genetic alterations being introduced with a vector includes the NIN or NLP protein being a NLP4 orthogroup protein and having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group of SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:391, SEQ ID NO:392, SEQ ID NO:393, SEQ ID NO:394, SEQ ID NO:395, SEQ ID NO:396, SEQ ID NO:397, SEQ ID NO:398, SEQ ID NO:399, SEQ ID NO:400, SEQ ID NO:401, SEQ ID NO:402, SEQ ID NO:403, SEQ ID NO:404, SEQ ID NO:405, SEQ ID NO:406, SEQ ID NO:408, SEQ ID NO:409, SEQ ID NO:410, SEQ ID NO:411, SEQ ID NO:412, SEQ ID NO:413, SEQ ID NO:414, SEQ ID NO:415, SEQ ID NO:417, SEQ ID NO:418, SEQ ID NO:419, SEQ ID NO:420, SEQ ID NO:421, SEQ ID NO:422, SEQ ID NO:423, SEQ ID NO:424, SEQ ID NO:425, SEQ ID NO:426, SEQ ID NO:427, SEQ ID NO:428, SEQ ID NO:429, SEQ ID NO:430, SEQ ID NO:431, SEQ ID NO:432, SEQ ID NO:433, SEQ ID NO:434, SEQ ID NO:435, SEQ ID NO:436, SEQ ID NO:437, SEQ ID NO:438, SEQ ID NO:439, SEQ ID NO:440, SEQ ID NO:441, SEQ ID NO:442, SEQ ID NO:443, SEQ ID NO:444, SEQ ID NO:445, SEQ ID NO:446, SEQ ID NO:447, SEQ ID NO:448, SEQ ID NO:449, SEQ ID NO:450, SEQ ID NO:451, SEQ ID NO:452, SEQ ID NO:453, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, SEQ ID NO:484, SEQ ID NO:485, SEQ ID NO:486, SEQ ID NO:487, SEQ ID NO:488, SEQ ID NO:489, SEQ ID NO:490, SEQ ID NO:491, SEQ ID NO:492, SEQ ID NO:493, SEQ ID NO:494, SEQ ID NO:495, SEQ ID NO:496, SEQ ID NO:497, SEQ ID NO:498, SEQ ID NO:499, SEQ ID NO:500, SEQ ID NO:501, SEQ ID NO:502, SEQ ID NO:504, SEQ ID NO:505, SEQ ID NO:506, SEQ ID NO:507, SEQ ID NO:508, SEQ ID NO:509, SEQ ID NO:510, SEQ ID NO:511, SEQ ID NO:512, SEQ ID NO:513, SEQ ID NO:514, SEQ ID NO:515, SEQ ID NO:516, SEQ ID NO:517, SEQ ID NO:518, SEQ ID NO:519, SEQ ID NO:520, SEQ ID NO:521, SEQ ID NO:522, SEQ ID NO:523, and SEQ ID NO:524. An additional embodiment of this present aspect that can be combined with any of the preceding aspects that has genetic alterations being introduced with a vector includes the NIN or NLP protein being a basal NIN/NLP orthogroup protein and having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO:637, SEQ ID NO:638, SEQ ID NO:639, SEQ ID NO:640, SEQ ID NO:641, SEQ ID NO:642, SEQ ID NO:643, SEQ ID NO:644, SEQ ID NO:645, SEQ ID NO:646, SEQ ID NO:647, SEQ ID NO:648, SEQ ID NO:649, SEQ ID NO:650, SEQ ID NO:651, SEQ ID NO:652, SEQ ID NO:653, SEQ ID NO:654, SEQ ID NO:655, SEQ ID NO:656, SEQ ID NO:657, SEQ ID NO:658, SEQ ID NO:659, SEQ ID NO:660, SEQ ID NO:661, or SEQ ID NO:662.

A further aspect of the disclosure includes methods of cultivating the genetically altered plant of any of the preceding embodiments that has a genetically altered plant, including the steps of: planting a genetically altered seedling, a genetically altered plantlet, a genetically altered cutting, a genetically altered tuber, a genetically altered root, or a genetically altered seed in soil to produce the genetically altered plant or grafting the genetically altered seedling, the genetically altered plantlet, or the genetically altered cutting to a root stock or a second plant grown in soil to produce the genetically altered plant; cultivating the plant to produce harvestable seed, harvestable leaves, harvestable roots, harvestable cuttings, harvestable wood, harvestable fruit, harvestable kernels, harvestable tubers, and/or harvestable grain; and harvesting the harvestable seed, harvestable leaves, harvestable roots, harvestable cuttings, harvestable wood, harvestable fruit, harvestable kernels, harvestable tubers, and/or harvestable grain.

An aspect of the disclosure includes a genetically altered plant, wherein the plant or a part thereof includes one or more genetic alterations that increase activity of a NODULE INCEPTION (NIN) protein or a NIN-like protein (NLP protein) in response to cytokinin signaling as compared to a wild type (WT) plant without the one or more genetic alterations, and wherein the plant or the part thereof includes a nucleic acid encoding the NIN protein or the NLP protein. An additional embodiment of this aspect includes the one or more genetic alterations being addition of one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, or twenty-four or more cytokinin response elements operably linked to the nucleic acid encoding the NIN protein or the NLP protein. Yet another embodiment of this aspect includes at least one of the cytokinin response elements being a B-type cytokinin signaling RESPONSE REGULATOR (RR) binding site. A further embodiment of this aspect includes at least one of the B-type cytokinin signaling RR binding sites having the sequence of SEQ ID NO:613 or SEQ ID NO:614. Yet another embodiment of this aspect includes at least one of the B-type cytokinin signaling RR binding sites having the sequence of SEQ ID NO:679, SEQ ID NO:680, SEQ ID NO:681, SEQ ID NO:682, SEQ ID NO:683, SEQ ID NO:684, SEQ ID NO:685, or SEQ ID NO:686. Still another embodiment of this aspect includes at least one of the B-type cytokinin signaling RR binding sites having the sequence selected from the group of SEQ ID NO:551, SEQ ID NO:552, SEQ ID NO:553, SEQ ID NO:554, SEQ ID NO:555, SEQ ID NO:556, SEQ ID NO:557, SEQ ID NO:558, SEQ ID NO:559, SEQ ID NO:560, SEQ ID NO:561, SEQ ID NO:562, SEQ ID NO:563, SEQ ID NO:564, SEQ ID NO:565, SEQ ID NO:566, SEQ ID NO:567, SEQ ID NO:568, SEQ ID NO:569, SEQ ID NO:570, SEQ ID NO:571, SEQ ID NO:572, SEQ ID NO:573, SEQ ID NO:574, SEQ ID NO:575, SEQ ID NO:576, SEQ ID NO:577, SEQ ID NO:578, SEQ ID NO:579, SEQ ID NO:580, SEQ ID NO:581, SEQ ID NO:582, SEQ ID NO:583, SEQ ID NO:584, SEQ ID NO:585, SEQ ID NO:586, SEQ ID NO:587, SEQ ID NO:588, SEQ ID NO:589, SEQ ID NO:590, SEQ ID NO:591, SEQ ID NO:592, SEQ ID NO:593, SEQ ID NO:594, SEQ ID NO:595, SEQ ID NO:596, SEQ ID NO:597, SEQ ID NO:598, SEQ ID NO:599, SEQ ID NO:600, SEQ ID NO:601, SEQ ID NO:602, SEQ ID NO:603, SEQ ID NO:604, SEQ ID NO:605, SEQ ID NO:606, SEQ ID NO:607, SEQ ID NO:608, SEQ ID NO:609, SEQ ID NO:610, SEQ ID NO:611, SEQ ID NO:612, SEQ ID NO:615, SEQ ID NO:616, SEQ ID NO:617, SEQ ID NO:618, SEQ ID NO:619, SEQ ID NO:620, SEQ ID NO:621, SEQ ID NO:622, SEQ ID NO:623, SEQ ID NO:624, SEQ ID NO:625, SEQ ID NO:626, SEQ ID NO:679, SEQ ID NO:680, SEQ ID NO:681, SEQ ID NO:682, SEQ ID NO:683, SEQ ID NO:684, SEQ ID NO:685, or SEQ ID NO:686.

In yet another embodiment, which may be combined with any of the preceding embodiments, the cytokinin response elements are within 100 nucleotides, within 90 nucleotides, within 86 nucleotides, within 80 nucleotides, within 70 nucleotides, within 60 nucleotides, within 50 nucleotides, within 40 nucleotides, within 30 nucleotides, within 25 nucleotides, within 20 nucleotides, within 19 nucleotides, within 18 nucleotides, within 17 nucleotides, within 16 nucleotides, within 15 nucleotides, within 14 nucleotides, within 13 nucleotides, within 12 nucleotides, within 11 nucleotides, within 10 nucleotides, within 9 nucleotides, within 8 nucleotides, within 7 nucleotides, within 6 nucleotides, within 5 nucleotides, within 4 nucleotides, within 3 nucleotides, within 2 nucleotides, or within 1 nucleotide of each other. In an additional embodiment of this aspect, the cytokinin response elements are within 11 nucleotides of each other. In yet another embodiment, which may be combined with any of the preceding embodiments, the nucleic acid encoding the NIN protein or the NLP protein is operably linked to a promoter that is operably linked to the cytokinin response elements. In an additional embodiment of this aspect, the promoter and the cytokinin response elements are within 110,000 nucleotides, within 105,000 nucleotides, within 100,000 nucleotides, within 95,000 nucleotides, within 90,000 nucleotides, within 85,000 nucleotides, within 80,000 nucleotides, within 75,000 nucleotides, within 70,000 nucleotides, within 65,000 nucleotides, within 60,000 nucleotides, within 55,000 nucleotides, within 50,000 nucleotides, within 45,000 nucleotides, within 42,000 nucleotides, within 40,000 nucleotides, within 35,000 nucleotides, within 30,000 nucleotides, within 25,000 nucleotides, within 20,000 nucleotides, within 15,000 nucleotides, within 10,000 nucleotides, within 9,000 nucleotides, within 8,000 nucleotides, within 7,000 nucleotides, within 6,000 nucleotides, within 5,000 nucleotides, within 4,000 nucleotides, within 3,000 nucleotides, within 2,000 nucleotides, within 1,000 nucleotides, within 500 nucleotides, within 400 nucleotides, within 300 nucleotides, within 200 nucleotides, or within 100 nucleotides of each other.

Still another embodiment of this aspect that can be combined with any of the preceding embodiments includes the nucleic acid encoding a NIN/NLP1 orthogroup protein. An additional embodiment of this aspect includes the NIN/NLP1 orthogroup protein having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group of SEQ ID NO:1, SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22; SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:108, SEQ ID NO:109, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:687, SEQ ID NO:688, SEQ ID NO:689, SEQ ID NO:690, SEQ ID NO:691, SEQ ID NO:692, or SEQ ID NO:693. A further embodiment of this aspect includes the NIN/NLP1 orthogroup protein being a NIN protein and having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group of SEQ ID NO:22 (i.e., CsaNIN; Cannabis sativa), SEQ ID NO:78 (i.e., HluNIN; Humulus lupulus), SEQ ID NO:89 (i.e., LjNIN; Lotus japonicus), SEQ ID NO:108 (i.e., MtNIN; Medicago truncatula); SEQ ID NO:136 (i.e., PanNIN; Parasponia andersonii), SEQ ID NO:139 (i.e., PriNIN; Parasponia rigida), SEQ ID NO:142 (i.e., PruNIN; Parasponia rugosa), SEQ ID NO:185 (i.e., TleNIN; Trema levigata), SEQ ID NO:187 (i.e., TorNIN; Trema orientalis), SEQ ID NO:190 (i.e., TtoNIN; Trema tomentosa), SEQ ID NO:236 (i.e., ZjuNIN; Ziziphus jujuba), SEQ ID NO:687 (i.e., AglNIN; Alnus glutinosa), SEQ ID NO:688 (i.e., CglNIN; Casuarina glauca), SEQ ID NO:689 (i.e., DglNIN.1; Datisca glomerata), SEQ ID NO:690 (i.e., DglNIN.2; Datisca glomerata), SEQ ID NO:691 (i.e., DtrNIN; Discaria trinervis), SEQ ID NO:692 (i.e., DdrNIN; Dryas drummondii), or SEQ ID NO:693 (i.e., PtrNIN; Purshia tridentata). Still another embodiment of this aspect includes the NIN/NLP1 orthogroup protein being a NIN protein and having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group of SEQ ID NO:89 (i.e., LjNIN; Lotus japonicus) or SEQ ID NO:108 (i.e., MtNIN; Medicago truncatula).

Yet another embodiment of this aspect that can be combined with any of the preceding embodiments that has a genetically altered plant includes the nucleic acid encoding a NLP2-3 orthogroup protein, a NLP4 orthogroup protein, or a basal NIN/NLP orthogroup protein. An additional embodiment of this aspect includes the NLP2-3 orthogroup protein having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group of SEQ ID NO:237, SEQ ID NO:238, SEQ ID NO:239, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, SEQ ID NO:282, SEQ ID NO:283, SEQ ID NO:284, SEQ ID NO:285, SEQ ID NO:286, SEQ ID NO:287, SEQ ID NO:288, SEQ ID NO:289, SEQ ID NO:290, SEQ ID NO:291, SEQ ID NO:292, SEQ ID NO:293, SEQ ID NO:294, SEQ ID NO:295, SEQ ID NO:296, SEQ ID NO:297, SEQ ID NO:298, SEQ ID NO:299, SEQ ID NO:300, SEQ ID NO:301, SEQ ID NO:302, SEQ ID NO:303, SEQ ID NO:304, SEQ ID NO:305, SEQ ID NO:306, SEQ ID NO:307, SEQ ID NO:308, SEQ ID NO:309, SEQ ID NO:310, SEQ ID NO:311, SEQ ID NO:312, SEQ ID NO:313, SEQ ID NO:314, SEQ ID NO:315, SEQ ID NO:316, SEQ ID NO:317, SEQ ID NO:318, SEQ ID NO:319, SEQ ID NO:320, SEQ ID NO:321, SEQ ID NO:322, SEQ ID NO:323, SEQ ID NO:324, SEQ ID NO:325, SEQ ID NO:326, SEQ ID NO:327, SEQ ID NO:328, SEQ ID NO:329, SEQ ID NO:332, SEQ ID NO:333, SEQ ID NO:334, SEQ ID NO:335, SEQ ID NO:336, SEQ ID NO:337, SEQ ID NO:338, SEQ ID NO:339, SEQ ID NO:340, SEQ ID NO:341, SEQ ID NO:342, SEQ ID NO:343, SEQ ID NO:344, SEQ ID NO:345, SEQ ID NO:346, SEQ ID NO:347, SEQ ID NO:348, SEQ ID NO:349, SEQ ID NO:350, SEQ ID NO:351, SEQ ID NO:352, SEQ ID NO:353, SEQ ID NO:354, SEQ ID NO:355, SEQ ID NO:356, SEQ ID NO:357, SEQ ID NO:358, SEQ ID NO:359, SEQ ID NO:360, SEQ ID NO:361, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:371, SEQ ID NO:372, SEQ ID NO:373, SEQ ID NO:374, SEQ ID NO:375, SEQ ID NO:376, and SEQ ID NO:377. Yet another embodiment of this aspect includes the NLP4 orthogroup protein having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group of SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:391, SEQ ID NO:392, SEQ ID NO:393, SEQ ID NO:394, SEQ ID NO:395, SEQ ID NO:396, SEQ ID NO:397, SEQ ID NO:398, SEQ ID NO:399, SEQ ID NO:400, SEQ ID NO:401, SEQ ID NO:402, SEQ ID NO:403, SEQ ID NO:404, SEQ ID NO:405, SEQ ID NO:406, SEQ ID NO:408, SEQ ID NO:409, SEQ ID NO:410, SEQ ID NO:411, SEQ ID NO:412, SEQ ID NO:413, SEQ ID NO:414, SEQ ID NO:415, SEQ ID NO:417, SEQ ID NO:418, SEQ ID NO:419, SEQ ID NO:420, SEQ ID NO:421, SEQ ID NO:422, SEQ ID NO:423, SEQ ID NO:424, SEQ ID NO:425, SEQ ID NO:426, SEQ ID NO:427, SEQ ID NO:428, SEQ ID NO:429, SEQ ID NO:430, SEQ ID NO:431, SEQ ID NO:432, SEQ ID NO:433, SEQ ID NO:434, SEQ ID NO:435, SEQ ID NO:436, SEQ ID NO:437, SEQ ID NO:438, SEQ ID NO:439, SEQ ID NO:440, SEQ ID NO:441, SEQ ID NO:442, SEQ ID NO:443, SEQ ID NO:444, SEQ ID NO:445, SEQ ID NO:446, SEQ ID NO:447, SEQ ID NO:448, SEQ ID NO:449, SEQ ID NO:450, SEQ ID NO:451, SEQ ID NO:452, SEQ ID NO:453, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, SEQ ID NO:484, SEQ ID NO:485, SEQ ID NO:486, SEQ ID NO:487, SEQ ID NO:488, SEQ ID NO:489, SEQ ID NO:490, SEQ ID NO:491, SEQ ID NO:492, SEQ ID NO:493, SEQ ID NO:494, SEQ ID NO:495, SEQ ID NO:496, SEQ ID NO:497, SEQ ID NO:498, SEQ ID NO:499, SEQ ID NO:500, SEQ ID NO:501, SEQ ID NO:502, SEQ ID NO:504, SEQ ID NO:505, SEQ ID NO:506, SEQ ID NO:507, SEQ ID NO:508, SEQ ID NO:509, SEQ ID NO:510, SEQ ID NO:511, SEQ ID NO:512, SEQ ID NO:513, SEQ ID NO:514, SEQ ID NO:515, SEQ ID NO:516, SEQ ID NO:517, SEQ ID NO:518, SEQ ID NO:519, SEQ ID NO:520, SEQ ID NO:521, SEQ ID NO:522, SEQ ID NO:523, and SEQ ID NO: 524. A further embodiment of this aspect includes the basal NIN/NLP orthogroup protein having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group of SEQ ID NO:637, SEQ ID NO:638, SEQ ID NO:639, SEQ ID NO:640, SEQ ID NO:641, SEQ ID NO:642, SEQ ID NO:643, SEQ ID NO:644, SEQ ID NO:645, SEQ ID NO:646, SEQ ID NO:647, SEQ ID NO:648, SEQ ID NO:649, SEQ ID NO:650, SEQ ID NO:651, SEQ ID NO:652, SEQ ID NO:653, SEQ ID NO:654, SEQ ID NO:655, SEQ ID NO:656, SEQ ID NO:657, SEQ ID NO:658, SEQ ID NO:659, SEQ ID NO:660, SEQ ID NO:661, or SEQ ID NO:662.

In yet another embodiment, which may be combined with any of the preceding embodiments, the nucleic acid encoding the NIN protein or the NLP protein is endogenous. Still another embodiment of this aspect that can be combined with any of the preceding embodiments includes the nucleic acid encoding the NIN protein or the NLP protein being heterologous. Yet another embodiment of this present aspect that can be combined with any of the preceding aspects that has the nucleic acid encoding the NIN protein or the NLP protein operably linked to a promoter includes the promoter being endogenous. Still another embodiment of this aspect that can be combined with any of the preceding aspects that has the nucleic acid encoding the NIN protein or the NLP protein operably linked to a promoter includes the promoter being heterologous.

In still another embodiment, which may be combined with any of the preceding embodiments, cytokinin signaling or induction of the cytokinin signaling pathway in a root pericycle cell layer, a root endodermis cell layer (i.e., endodermal cell layer), root cortex cell layers (i.e., cortical cell layer), and/or a root epidermis cell layer (i.e., epidermal cell layer) induces nodule organogenesis. Still another embodiment of this aspect that can be combined with any of the preceding embodiments further includes one or more CYCLOPS response elements operably linked to the nucleic acid. An additional embodiment of this aspect includes CYCLOPS expression in a root epidermis cell layer (i.e., epidermal cell layer) inducing rhizobium infection.

In a further embodiment, which may be combined with any of the preceding embodiments, the genetically altered plant is a monocot. An additional embodiment of this aspect includes the genetically altered plant being selected from the group of corn, rice, wheat, barley, sorghum, millet, oat, or rye. Still another embodiment of this aspect that can be combined with any of the preceding embodiments further includes the genetically altered plant being selected from the group of apple, pear, plum, apricot, peach, almond, walnut, cherry, strawberry, raspberry, blackberry, red currant, black currant, melon, cucumber, pumpkin, squash, grape, hemp, hops, birch, beech, jujube, cassava, poplar, chestnut, citrus, potato, tomato, sweet potato, Trema spp., and Jatropha spp. In yet another embodiment, which may be combined with any of the preceding embodiments, the WT plant is not a legume, does not form nodules for symbiosis with nitrogen fixing bacteria, or both is not a legume and does not form nodules for symbiosis with nitrogen fixing bacteria.

Still another embodiment of this aspect that can be combined with any of the preceding embodiments includes a genetically altered plant part of the genetically altered plant of any one of the preceding embodiments with respect to plant parts, wherein the plant part is a leaf, a stem, a root, a tuber, a flower, a seed, a kernel, a grain, a fruit, a cell, or a portion thereof and the genetically altered plant part includes the one or more genetic alterations. An additional embodiment of this aspect includes the plant part being a fruit, a tuber, a kernel, or a grain. Yet another embodiment of this aspect that can be combined with any of the preceding embodiments with respect to pollen grain or ovules includes a genetically altered pollen grain or a genetically altered ovule of the plant of any one of the preceding embodiments, wherein the genetically altered pollen grain or the genetically altered ovule includes the one or more genetic alterations. A further embodiment of this aspect that can be combined with any of the preceding embodiments includes a genetically altered protoplast produced from the genetically altered plant of any of the preceding embodiments, wherein the genetically altered protoplast includes the one or more genetic alterations. An additional embodiment of this aspect that can be combined with any of the preceding embodiments includes a genetically altered tissue culture produced from protoplasts or cells from the genetically altered plant of any one of the preceding embodiments, wherein the cells or protoplasts are produced from a plant part selected from the group of leaf, leaf mesophyll cell, anther, pistil, stem, petiole, root, root tip, tuber, fruit, seed, kernel, grain, flower, cotyledon, hypocotyl, embryo, or meristematic cell, wherein the genetically altered tissue culture includes the one or more genetic alterations. An additional embodiment of this aspect includes a genetically altered plant regenerated from the genetically altered tissue culture that includes the one or more genetic alterations. Still another embodiment of this aspect that can be combined with any of the preceding embodiments that has a genetically altered plant includes the genetically altered plant having all the physiological and morphological characteristics of the plant of any of the preceding embodiments. Yet another embodiment of this aspect that can be combined with any of the preceding embodiments that has a genetically altered plant includes a genetically altered plant seed produced from the genetically altered plant of any one of the preceding embodiments. A further embodiment of this aspect that can be combined with any of the preceding embodiments that has a genetically altered plant includes the seed of the plant producing a plant having all the physiological and morphological characteristics of the plant of any of the above embodiments.

An additional aspect of the disclosure includes methods of producing the genetically altered plant of any of the preceding embodiments that has a genetically altered plant, including the steps of: (a) introducing the one or more genetic alterations into a plant cell, tissue, or other explant; (b) regenerating the plant cell, tissue, or other explant into a genetically altered plantlet; and (c) growing the genetically altered plantlet into a genetically altered plant with the one or more genetic alterations that increase activity of the NIN protein or the NLP protein in response to cytokinin signaling as compared to an untransformed WT plant. An additional embodiment of this aspect further includes identifying successful introduction of the one or more genetic alterations by screening or selecting the plant cell, tissue, or other explant prior to step (b); screening or selecting plantlets between step (b) and (c); or screening or selecting plants after step (c). In yet another embodiment, which may be combined with any of the preceding embodiments, transformation is done using a transformation method selected from the group of particle bombardment (i.e., biolistics, gene gun), Agrobacterium-mediated transformation, Rhizobium-mediated transformation, or protoplast transfection or transformation.

Still another embodiment of this aspect that can be combined with any of the preceding embodiments includes genetic alterations being introduced with a vector. An additional embodiment of this aspect includes the vector including a promoter operably linked to a nucleotide encoding a NIN or NLP protein and one or more cytokinin response elements operably linked to the promoter. Yet another embodiment of this aspect includes the promoter and the one or more cytokinin response elements being selected from the group of a NIN gene promoter including a 5′-upstream sequence including a CYCLOPS response element through a transcription start site of the NIN gene operably linked to a 3C region, the NIN gene promoter comprising a 5′-upstream sequence including the CYCLOPS response element through to the transcription start site of the NIN gene operably linked to a CE region, a minimal promoter operably linked to a CYCLOPS response element operably linked to a CE region, and a minimal promoter operably linked to a CYCLOPS response element operably linked to one or more cytokinin response elements. In a further embodiment of this aspect, the vector includes one or more gene editing components that target a nuclear genome sequence operably linked to an endogenous NIN protein or NLP protein. Yet another embodiment of this aspect includes the nuclear genome sequence being edited by the one or more gene editing components to introduce a cis-regulatory element selected from the group of one or more cytokinin response elements, a 3C region, or a CE region. Yet another embodiment of this present aspect that can be combined with any of the preceding aspects that has a vector including one or more gene editing components includes one or more gene editing components being selected from the group of a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (ODN), wherein the ODN targets the nuclear genome sequence; or a vector including a CRISPR/Cas enzyme encoding sequence and a targeting sequence, wherein the targeting sequence targets the nuclear genome sequence.

A further embodiment of this present aspect that can be combined with any of the preceding aspects that has genetic alterations being introduced with a vector includes the NIN gene promoter, 3C region, CE region, CYCLOPS response element, or one or more cytokinin response elements being from a nodulating legume species. An additional embodiment of this aspect includes the nodulating legume species being selected from the group of peanut, pigeon pea, chickpea, soybean, velvet bean, bean, pea, adzuki bean, mung bean, clover, lupine, Lotus japonicus, and Medicago truncatula. Yet another embodiment of this present aspect that can be combined with any of the preceding aspects that has genetic alterations being introduced with a vector wherein the vector includes the NIN gene promoter, 3C region, CE region, CYCLOPS response element, or one or more cytokinin response elements from a nodulating legume species includes cytokinin response elements being selected from the group of SEQ ID NO:551, SEQ ID NO:552, SEQ ID NO:553, SEQ ID NO:554, SEQ ID NO:555, SEQ ID NO:556, SEQ ID NO:557, SEQ ID NO:558, SEQ ID NO:559, SEQ ID NO:560, SEQ ID NO:561, SEQ ID NO:562, SEQ ID NO:563, SEQ ID NO:564, SEQ ID NO:565, SEQ ID NO:566, SEQ ID NO:567, SEQ ID NO:568, SEQ ID NO:569, SEQ ID NO:570, SEQ ID NO:571, SEQ ID NO:572, SEQ ID NO:573, SEQ ID NO:574, SEQ ID NO:575, SEQ ID NO:576, SEQ ID NO:577, SEQ ID NO:578, SEQ ID NO:579, SEQ ID NO:580, SEQ ID NO:581, SEQ ID NO:582, SEQ ID NO:583, SEQ ID NO:584, SEQ ID NO:585, SEQ ID NO:586, SEQ ID NO:587, SEQ ID NO:588, SEQ ID NO:589, SEQ ID NO:590, SEQ ID NO:591, SEQ ID NO:592, SEQ ID NO:593, SEQ ID NO:594, SEQ ID NO:595, SEQ ID NO:596, SEQ ID NO:597, SEQ ID NO:598, SEQ ID NO:599, SEQ ID NO:600, SEQ ID NO:601, SEQ ID NO:602, SEQ ID NO:603, SEQ ID NO:604, SEQ ID NO:605, SEQ ID NO:606, SEQ ID NO:607, SEQ ID NO:608, SEQ ID NO:609, SEQ ID NO:610, SEQ ID NO:611, or SEQ ID NO:612. Still another embodiment of this present aspect that can be combined with any of the preceding aspects that has genetic alterations being introduced with a vector includes cytokinin response elements being selected from the group of SEQ ID NO:613, SEQ ID NO:614, SEQ ID NO:615, SEQ ID NO:616, SEQ ID NO:617, SEQ ID NO:618, SEQ ID NO:619, SEQ ID NO:620, SEQ ID NO:621, SEQ ID NO:622, SEQ ID NO:623, SEQ ID NO:624, SEQ ID NO:625, or SEQ ID NO:626. Yet another embodiment of this present aspect that can be combined with any of the preceding aspects that has genetic alterations being introduced with a vector includes one or more cytokinin response elements being from a non-nodulating species. In yet another embodiment of this present aspect that can be combined with any of the preceding aspects that has genetic alterations being introduced with a vector, the one or more cytokinin response elements from a non-nodulating species is SEQ ID NO:613. A further embodiment of this present aspect that can be combined with any of the preceding aspects that has genetic alterations being introduced with a vector includes the one or more cytokinin response elements being from a nodulating non-legume species. An additional embodiment of this present aspect that can be combined with any of the preceding aspects that has genetic alterations being introduced with a vector includes cytokinin response elements being selected from the group of SEQ ID NO:613, SEQ ID NO:614, SEQ ID NO:679, SEQ ID NO:680, SEQ ID NO:681, SEQ ID NO:682, SEQ ID NO:683, SEQ ID NO:684, SEQ ID NO:685, or SEQ ID NO:686.

A further embodiment of this present aspect that can be combined with any of the preceding aspects that has genetic alterations being introduced with a vector includes the NIN or NLP protein being a NIN/NLP1 orthogroup protein and having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group of SEQ ID NO:1, SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22; SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:108, SEQ ID NO:109, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:687, SEQ ID NO:688, SEQ ID NO:689, SEQ ID NO:690, SEQ ID NO:691, SEQ ID NO:692, or SEQ ID NO:693. Yet another embodiment of this present aspect that can be combined with any of the preceding aspects that has genetic alterations being introduced with a vector includes the NIN/NLP1 orthogroup protein being a NIN protein and having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group of SEQ ID NO:22 (i.e., CsaNIN; Cannabis sativa), SEQ ID NO:78 (i.e., HluNIN; Humulus lupulus), SEQ ID NO:89 (i.e., LjNIN; Lotus japonicus), SEQ ID NO:108 (i.e., MtNIN; Medicago truncatula); SEQ ID NO:136 (i.e., PanNIN; Parasponia andersonii), SEQ ID NO:139 (i.e., PriNIN; Parasponia rigida), SEQ ID NO:142 (i.e., PruNIN; Parasponia rugosa), SEQ ID NO:185 (i.e., TleNIN; Trema levigata), SEQ ID NO:187 (i.e., TorNIN; Trema orientalis), SEQ ID NO:190 (i.e., TtoNIN; Trema tomentosa), and SEQ ID NO:236 (i.e., ZjuNIN; Ziziphus jujuba), SEQ ID NO:687 (i.e., AglNIN; Alnus glutinosa), SEQ ID NO:688 (i.e., CglNIN; Casuarina glauca), SEQ ID NO:689 (i.e., DglNIN.1; Datisca glomerata), SEQ ID NO:690 (i.e., DglNIN.2; Datisca glomerata), SEQ ID NO:691 (i.e., DtrNIN; Discaria trinervis), SEQ ID NO:692 (i.e., DdrNIN; Dryas drummondii), or SEQ ID NO:693 (i.e., PtrNIN; Purshia tridentata). A further embodiment of this present aspect that can be combined with any of the preceding aspects that has genetic alterations being introduced with a vector includes the NIN/NLP1 orthogroup protein being a NIN protein and having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group of SEQ ID NO:89 (i.e., LjNIN; Lotus japonicus) or SEQ ID NO:108 (i.e., MtNIN; Medicago truncatula). Still another embodiment of this present aspect that can be combined with any of the preceding aspects that has genetic alterations being introduced with a vector includes the NIN or NLP protein being a NLP2-3 orthogroup protein and having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group of SEQ ID NO:237, SEQ ID NO:238, SEQ ID NO:239, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, SEQ ID NO:282, SEQ ID NO:283, SEQ ID NO:284, SEQ ID NO:285, SEQ ID NO:286, SEQ ID NO:287, SEQ ID NO:288, SEQ ID NO:289, SEQ ID NO:290, SEQ ID NO:291, SEQ ID NO:292, SEQ ID NO:293, SEQ ID NO:294, SEQ ID NO:295, SEQ ID NO:296, SEQ ID NO:297, SEQ ID NO:298, SEQ ID NO:299, SEQ ID NO:300, SEQ ID NO:301, SEQ ID NO:302, SEQ ID NO:303, SEQ ID NO:304, SEQ ID NO:305, SEQ ID NO:306, SEQ ID NO:307, SEQ ID NO:308, SEQ ID NO:309, SEQ ID NO:310, SEQ ID NO:311, SEQ ID NO:312, SEQ ID NO:313, SEQ ID NO:314, SEQ ID NO:315, SEQ ID NO:316, SEQ ID NO:317, SEQ ID NO:318, SEQ ID NO:319, SEQ ID NO:320, SEQ ID NO:321, SEQ ID NO:322, SEQ ID NO:323, SEQ ID NO:324, SEQ ID NO:325, SEQ ID NO:326, SEQ ID NO:327, SEQ ID NO:328, SEQ ID NO:329, SEQ ID NO:332, SEQ ID NO:333, SEQ ID NO:334, SEQ ID NO:335, SEQ ID NO:336, SEQ ID NO:337, SEQ ID NO:338, SEQ ID NO:339, SEQ ID NO:340, SEQ ID NO:341, SEQ ID NO:342, SEQ ID NO:343, SEQ ID NO:344, SEQ ID NO:345, SEQ ID NO:346, SEQ ID NO:347, SEQ ID NO:348, SEQ ID NO:349, SEQ ID NO:350, SEQ ID NO:351, SEQ ID NO:352, SEQ ID NO:353, SEQ ID NO:354, SEQ ID NO:355, SEQ ID NO:356, SEQ ID NO:357, SEQ ID NO:358, SEQ ID NO:359, SEQ ID NO:360, SEQ ID NO:361, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:371, SEQ ID NO:372, SEQ ID NO:373, SEQ ID NO:374, SEQ ID NO:375, SEQ ID NO:376, and SEQ ID NO:377. A further embodiment of this present aspect that can be combined with any of the preceding aspects that has genetic alterations being introduced with a vector includes the NIN or NLP protein being a NLP4 orthogroup protein and having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group of SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:391, SEQ ID NO:392, SEQ ID NO:393, SEQ ID NO:394, SEQ ID NO:395, SEQ ID NO:396, SEQ ID NO:397, SEQ ID NO:398, SEQ ID NO:399, SEQ ID NO:400, SEQ ID NO:401, SEQ ID NO:402, SEQ ID NO:403, SEQ ID NO:404, SEQ ID NO:405, SEQ ID NO:406, SEQ ID NO:408, SEQ ID NO:409, SEQ ID NO:410, SEQ ID NO:411, SEQ ID NO:412, SEQ ID NO:413, SEQ ID NO:414, SEQ ID NO:415, SEQ ID NO:417, SEQ ID NO:418, SEQ ID NO:419, SEQ ID NO:420, SEQ ID NO:421, SEQ ID NO:422, SEQ ID NO:423, SEQ ID NO:424, SEQ ID NO:425, SEQ ID NO:426, SEQ ID NO:427, SEQ ID NO:428, SEQ ID NO:429, SEQ ID NO:430, SEQ ID NO:431, SEQ ID NO:432, SEQ ID NO:433, SEQ ID NO:434, SEQ ID NO:435, SEQ ID NO:436, SEQ ID NO:437, SEQ ID NO:438, SEQ ID NO:439, SEQ ID NO:440, SEQ ID NO:441, SEQ ID NO:442, SEQ ID NO:443, SEQ ID NO:444, SEQ ID NO:445, SEQ ID NO:446, SEQ ID NO:447, SEQ ID NO:448, SEQ ID NO:449, SEQ ID NO:450, SEQ ID NO:451, SEQ ID NO:452, SEQ ID NO:453, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, SEQ ID NO:484, SEQ ID NO:485, SEQ ID NO:486, SEQ ID NO:487, SEQ ID NO:488, SEQ ID NO:489, SEQ ID NO:490, SEQ ID NO:491, SEQ ID NO:492, SEQ ID NO:493, SEQ ID NO:494, SEQ ID NO:495, SEQ ID NO:496, SEQ ID NO:497, SEQ ID NO:498, SEQ ID NO:499, SEQ ID NO:500, SEQ ID NO:501, SEQ ID NO:502, SEQ ID NO:504, SEQ ID NO:505, SEQ ID NO:506, SEQ ID NO:507, SEQ ID NO:508, SEQ ID NO:509, SEQ ID NO:510, SEQ ID NO:511, SEQ ID NO:512, SEQ ID NO:513, SEQ ID NO:514, SEQ ID NO:515, SEQ ID NO:516, SEQ ID NO:517, SEQ ID NO:518, SEQ ID NO:519, SEQ ID NO:520, SEQ ID NO:521, SEQ ID NO:522, SEQ ID NO:523, and SEQ ID NO:524. An additional embodiment of this present aspect that can be combined with any of the preceding aspects that has genetic alterations being introduced with a vector includes the NIN or NLP protein being a basal NIN/NLP orthogroup protein and having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group of SEQ ID NO:637, SEQ ID NO:638, SEQ ID NO:639, SEQ ID NO:640, SEQ ID NO:641, SEQ ID NO:642, SEQ ID NO:643, SEQ ID NO:644, SEQ ID NO:645, SEQ ID NO:646, SEQ ID NO:647, SEQ ID NO:648, SEQ ID NO:649, SEQ ID NO:650, SEQ ID NO:651, SEQ ID NO:652, SEQ ID NO:653, SEQ ID NO:654, SEQ ID NO:655, SEQ ID NO:656, SEQ ID NO:657, SEQ ID NO:658, SEQ ID NO:659, SEQ ID NO:660, SEQ ID NO:661, or SEQ ID NO:662.

A further aspect of the disclosure includes methods of cultivating the genetically altered plant of any of the preceding embodiments that has a genetically altered plant, including the steps of: planting a genetically altered seedling, a genetically altered plantlet, a genetically altered cutting, a genetically altered tuber, a genetically altered root, or a genetically altered seed in soil to produce the genetically altered plant or grafting the genetically altered seedling, the genetically altered plantlet, or the genetically altered cutting to a root stock or a second plant grown in soil to produce the genetically altered plant; cultivating the plant to produce harvestable seed, harvestable leaves, harvestable roots, harvestable cuttings, harvestable wood, harvestable fruit, harvestable kernels, harvestable tubers, and/or harvestable grain; and harvesting the harvestable seed, harvestable leaves, harvestable roots, harvestable cuttings, harvestable wood, harvestable fruit, harvestable kernels, harvestable tubers, and/or harvestable grain.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1I show the phenotype of Medicago truncatula daphne-like (FN8113) mutant roots and the phenotype of M. truncatula A17 wild type (WT) roots inoculated with Sinorhizobium meliloti (rhizobial bacteria) strain RCR2011.pHC60. FIG. 1A shows a stereo transmitted light macroscopy image of infection threads in an A17 WT root (scale bar 2 mm). FIG. 1B shows a stereo fluorescence macroscopy image of infection threads in an A17 WT root (scale bar 2 mm). FIG. 1D shows a stereo transmitted light macroscopy image of infection threads in a daphne-like mutant root (scale bar 2 mm). FIG. 1E shows a stereo fluorescence macroscopy image of infection threads in a daphne-like mutant root (scale bar 2 mm). The comparison of FIGS. 1A-1B with FIGS. 1D-1E shows that daphne-like mutant roots (FIGS. 1D-1E) have an excessive number of infection threads in comparison to WT (FIGS. 1A-1B). FIGS. 1C and 1F show confocal images of seven days post inoculation (dpi) WT (FIG. 1C; scale bar 10 μm) and daphne-like mutant (FIG. 1F; scale bar 10 μm) roots stained with propidium iodide, and show that bacterial colonies (arrow head) and infection threads (arrow) are comparable in daphne-like mutant roots and in WT roots. FIG. 1G shows a longitudinal section of a three weeks post inoculation (wpi) daphne-like mutant root stained with toluidine blue, which shows that the infection thread (arrow) can occasionally reach cortical cell layers and that some cell divisions are induced (arrow head) (scale bar 50 μm; ep=epidermis; C4=cortical cell layer 4; C5=cortical cell layer 5; ed=endodermis; pc=pericycle; infection threads indicated by arrow; cell divisions indicated by arrow head). FIG. 1H shows a schematic representation of the NIN locus in the daphne-like mutant, which has a 2.49 MB Chromosome 2 insert (flanking sequences shown between arrows are SEQ ID NO:633 and SEQ ID NO:634) 4120 bp upstream of the NIN gene (thick grey arrow) start codon (“ATG” on thin black arrow). From left to right, Chromosome 5 sequences are SEQ ID NO:632, SEQ ID NO:635, and SEQ ID NO:636. The images shown in FIGS. 1A-1G are representative images from multiple replicates. FIG. 1I shows mean±SD of data from confocal images of fourteen days post inoculation (dpi) roots stained with propidium iodide (representative images of these roots are shown in FIGS. 1C and 1F).

FIGS. 2A-2M show partial complementation of the infection process in M. truncatula nin-1 mutant roots by introducing the construct ProNIN_{5kb}:NIN, the construct ProNIN_{2.2kb}:NIN, or the construct ProNIN5kb(Δcyclops):NIN using Agrobacterium rhizogenes-mediated transformation. FIG. 2A shows a longitudinal section of a nin-1 mutant root transformed with ProNIN_{5kb}:NIN stained with toluidine blue displaying infection threads (arrows) that occasionally can reach cortical cell layers (scale bar 50 μm; C4=cortical cell layer 4; C5=cortical cell layer 5; ed=endodermis; pc=pericycle; infection threads indicated by arrow). FIG. 2B shows a stereo transmitted light macroscopy image of a nin-1 mutant root transformed with ProNIN_{5kb}:NIN displaying excessive infection thread formation (scale bar 2 mm). FIG. 2C shows a stereo macroscopy fluorescence image of a nin-1 mutant root transformed with ProNIN_{5kb}:NIN displaying excessive infection thread formation (scale bar 2 mm). FIG. 2D shows a confocal image of a nin-1 mutant root transformed with ProNIN_{5kb}:NIN soot stained with propidium iodide displaying infection thread formation (long white line) initiated in a curled root hair (scale bar 10 μm). FIG. 2E shows a stereo transmitted light macroscopy image of a nin-1 mutant root transformed with ProNIN_{2.2kb}:NIN displaying numerous curled root hairs (scale bar 2 mm). FIG. 2F shows a stereo macroscopy fluorescence image of nin-1 roots transformed with ProNIN_{2.2kb}:NIN displaying numerous curled root hairs (scale bar 2 mm). FIG. 2G shows a confocal image of a nin-1 mutant root transformed with ProNIN_{2.2kb}:NIN root stained with propidium iodide displaying a bacterial curl colony (compact white shape) inside a curled root hair, but no infection thread formation (scale bar 10 μm). FIG. 2H shows a stereo transmitted light macroscopy image of a nin-1 mutant root transformed with empty vector without infection threads (scale bar 2 mm). FIG. 2I shows a stereo macroscopy fluorescence image of a nin-1 mutant root transformed with empty vector without infection threads (scale bar 2 mm). FIG. 2J shows a confocal image of a nin-1 mutant root transformed with empty vector stained with propidium iodide displaying excessive root hair curling without a bacterial colony (scale bar 10 μm). FIG. 2K shows a stereo transmitted light macroscopy image of nin-1 roots transformed with ProNIN5kb(Δcyclops):NIN displaying numerous curled root hairs (scale bar 2 mm). FIG. 2L shows a stereo macroscopy fluorescence image of nin-1 roots transformed with ProNIN5kb(Δcyclops):NIN displaying numerous curled root hairs (scale bar 2 mm). FIG. 2M shows a confocal image of a nin-1 mutant root transformed with ProNIN5kb(Δcyclops):NIN root stained with propidium iodide displaying a bacterial curl colony (compact white shape) inside a curled root hair, but no infection thread formation (scale bar 10 μm). FIGS. 2A-2M show images of roots collected at 4 wpi inoculation with S. meliloti RCR2011.pHC60 constitutively expressing GFP. The images shown in FIGS. 2A-2M are representative images from multiple replicates.

FIG. 3 shows mVISTA alignment of genomic DNA sequences containing 2 kb downstream from the NIN gene start codon, and NIN 5′-upstream regions from 8 legume species. The x-axis provides the distance from the M. truncatula NIN start codon in Kb (running right to left), while the y-axis provides the percentage conservation level to the M. truncatula sequence (running bottom to top for each legume species). Peaks indicate the level of sequence identity with M. truncatula on a scale of 50%-100%, whereby identities lower than 50% were not scored. Sequences 2 kb downstream from the NIN start codon are depicted to the right of the thin black arrow labelled “ATG”, 5′-non-coding upstream DNA sequences are depicted to the left of the thin black arrow. The two dark grey rectangles indicate the locations of the 3 Conserved region (3C region; left) and the −5 kb promoter region (right), and the grey vertical arrow indicates the location of the CYCLOPS binding site within the −5 kb promoter region.

FIGS. 4A and 4B show a schematic representation of the elements in the M. truncatula NIN 5′-upstream region and experimental results demonstrating that the cytokinin response elements containing (CE) region is essential for nodule organogenesis. FIG. 4A shows the 3C region present in the NIN 5′-upstream region as boxes located −15 Kb to −20 Kb upstream of the NIN coding sequence start site (NIN gene=thick grey arrow; start codon=“ATG” on thin black arrow). The middle region of the 3C region is the 1 Kb CE region, which contains a 472 bp conserved region is divided into three parts or domains named D1, D2, and D3 (depicted by grey boxes). FIG. 4A also shows the location of the insertion in the daphne-like mutant and the location of the CYCLOPS binding site, which are shown as labelled arrows located between −2.2 Kb and −5 Kb upstream of the NIN coding sequence start site. FIG. 4B shows the number of nodules formed on A17 WT M. truncatula roots transformed with empty vector (top bar) and M. truncatula nin-1 mutant roots transformed with empty vector or constructs carrying the NIN gene driven by different parts of the NIN 5′-upstream region (bottom 6 bars; each bar is labelled with the specific construct used). The ratio of nodulated roots to total roots tested (indicated with an arrow labelled “Nodulated roots/transgenic roots) is provided on the left of the graph. The graph shows the number of nodules per nodulated root, data are mean±SD, and nodule numbers were counted at 4 wpi with S. meliloti strain 2011.pHC60.

FIGS. 5A-5D show MAFFT alignments of the conserved part of the cytokinin response elements containing (CE) region (corresponding to the second or middle region of the larger 3C region) and the CYCLOPS binding site of 8 legume species. FIGS. 5A-5C shows MAFFT alignment of the conserved part (i.e., without flanking regions) of the CE region of 8 legume species; Medicago truncatula (SEQ ID NO: 663), Trifolium pratense (SEQ ID NO: 664), Cicer arietinum (SEQ ID NO: 665), Lotus japonicus (SEQ ID NO: 666), Glycine max (SEQ ID NO: 667), Cajanus cajan (SEQ ID NO: 668), Lupinus angustifolius (SEQ ID NO: 669), and Arachis duranensis (SEQ ID NO: 670). The conserved part of the CE region contains about 10 putative B-type cytokinin signaling RESPONSE REGULATOR (RR) binding sites (SEQ ID NO:613, bold text), and one AP2 binding element (SEQ ID NO:631, surrounded by black box in FIG. 5B). The conserved part of the CE region is divided into three domains named D1, D2, and D3, whose extent and boundaries are indicated by black arrows beneath the alignment and vertical black lines through the alignment. FIG. 5A shows the alignment of the 5′ portion of the of the conserved part of the CE region, which contains all of domain D1 and part of domain D2. FIG. 5B shows the alignment of the central portion of the conserved part of the CE region, which contains part of domain D2 and part of domain D3. FIG. 5C shows the alignment of the 3′ portion of the conserved part of the CE region, which contains part of domain D3. FIG. 5D shows MAFFT alignment of the CYCLOPS binding site of 8 legume species; Medicago truncatula (SEQ ID NO: 671), Arachis duranensis (SEQ ID NO: 672), Cicer arietinum (SEQ ID NO: 673), Lotus japonicus (SEQ ID NO: 674), Glycine max (SEQ ID NO: 675), Lupinus angustifolius (SEQ ID NO: 676), Cajanus cajan (SEQ ID NO: 677), and Trifolium pratense (SEQ ID NO: 678). The two boxes outlined with a dashed line indicate the palindromic sequence of the essential cis element, which is referred to as CYC-box and is also shown by labelled black arrows above the alignment, within the CYCLOPS response element (also referred to as CYCLOPS responsive cis element or CYC-RE).

FIGS. 6A-6H show complementation of non-nodulation phenotypes of M. truncatula nin-1 and daphne-like mutant roots by introducing the construct ProNIN_{3C-5kb}:NIN, ProNIN_{CE-5kb}:NIN, or ProNIN_{CE-35Smin}:NIN using A. rhizogenes-mediated transformation (35 min=Minimal CaMV 35S promoter). FIGS. 6A and 6C show that nodules are formed on transgenic roots of nin-1 when transformed with ProNIN_{3C-5kb}:NIN (FIG. 6A; scale bar 2 mm) or when transformed with ProNIN_{CE-5kb}:NIN (FIG. 6C; scale bar 2 mm). The nodules in the images in color can be seen as pink, which indicates the nodules are actively fixing nitrogen. FIGS. 6E and 6G show that nodules are formed on transgenic roots of daphne-like when transformed with ProNIN_{CE-5kb}:NIN (FIG. 6E; scale bar 2 mm) or when transformed with ProNIN_{CE-35Smin}:NIN (FIG. 6G; scale bar 2 mm). The nodules in the images in color can be seen as pink, which indicates the nodules are actively fixing nitrogen. FIGS. 6B and 6D show longitudinal sections of the nodules that are formed on transgenic roots of nin-1 when transformed with ProNIN_{3C-5kb}:NIN (FIG. 6B; scale bar 200 μm) or when transformed with ProNIN_{CE-5kb}:NIN (FIG. 6D; scale bar 200 μm) stained with toluidine blue, which have normal nodule zonation, including meristem (M), infection zone (IF), and fixation zone (FX). FIGS. 6F and 6H show longitudinal sections of a nodule that are formed on transgenic roots of daphne-like when transformed with ProNIN_{CE-5kb}:NIN (FIG. 6F; scale bar 200 μm) or when transformed with ProNIN_{CE-35Smin}:NIN (FIG. 6H; scale bar 200 μm) stained with toluidine blue, which have normal nodule zonation, including meristem (M), infection zone (IF), and fixation zone (FX). In FIGS. 6A-6H, S. meliloti strain RCR2011 containing constitutively expressed GFP was used as inoculum, and nodules were collected at 4 wpi. The images shown in FIGS. 6A-6H are representative images from multiple replicates.

FIGS. 7A and 7B show nifH expression is induced in M. truncatula ProNIN_{CE-5kb}:NIN transgenic nin-1 root nodules when they are inoculated with S. meliloti carrying the PronifH:GFP reporter. FIG. 7A shows a confocal image of a 4 wpi transgenic nodule, which shows that nifH (light grey) is switched on in the fixation zone (FX) (IF=infection zone; scale bar 200 μm). FIG. 7B shows a close-up image of FIG. 7A showing nifH switched on in fixation zone (S=symbiosome; scale bar 50 μm). The images shown in FIGS. 7A-7B are representative images from multiple replicates.

FIGS. 8A-8B show qRT-PCR analysis of relative NIN and NF-YA1 expression in response to cytokinin induction as compared to a water control in A17 WT and daphne-like. FIG. 8A shows qRT-PCR analysis of relative expression of NIN in A17 WT and daphne-like after application of 10-7 M benzylaminopurine (BAP; indicated by the label “10⁻⁷ BAP”) for cytokinin induction or water (indicated by the label “H₂O”) as a control for 16 hours. FIG. 8B shows qRT-PCR analysis of relative expression of NF-YA1 in A17 WT and daphne-like after application of 10-7 M BAP for cytokinin induction or water as a control for 16 hours. FIGS. 8A-8B show means of three biological replicates with error bars indicating SEM.

FIGS. 9A-9C show the phenotype of M. truncatula nin-1 mutant roots transformed with ProNIN_{CE(ΔD1/D2/D3)-5kb}:NIN constructs. FIG. 9A shows a longitudinal section of inoculated nin-1 root transformed with ProNIN_{CE(ΔD1)-5kb}:NIN (scale bar 50 μm; ep=epidermis; C4=cortical cell layer 4; C5=cortical cell layer 5; ed=endodermis; pc=pericycle; infection thread indicated by arrow). FIG. 9B shows a longitudinal section of inoculated nin-1 root transformed with ProNIN_{CE(ΔD2)-5kb}:NIN (IF=infection zone; FX=fixation zone; M=meristem; scale bar 200 μm). FIG. 9C shows nodule sections of nin-1 transformed with ProNIN_{CE(ΔD3)-5kb}:NIN (IF=infection zone; FX=fixation zone; scale bar 200 μm). Sections in FIGS. 9A-9C are stained with toluidine blue. The images shown in FIGS. 9A-9C are representative images from multiple replicates.

FIGS. 10A-10E show NIN and NF-YA1 expression patterns in M. truncatula A17 WT nodule primordia and daphne-like mutant primordia inoculated with S. meliloti RCR2011. FIGS. 10A-10B show RNA in situ localization of NIN (FIG. 10A) and NF-YA1 (FIG. 10B) in A17 nodule primordia at a stage of nodule primordium development in which the pericycle cells had divided and some anticlinal divisions had occurred in the inner cortical cell layers (C4 and C5). FIGS. 10C-10D show RNA in situ localization of NIN (FIG. 10C) and NF-YA1 (FIG. 10D) in A17 nodule primordia at a later stage of nodule primordium development when cortical cells had divided more extensively. FIG. 10E shows RNA in situ localization of NIN in daphne-like mutant primordia at two days post inoculation with S. meliloti RCR2011. In FIGS. 10A-10E, hybridization signals are depicted as dark dots and indicated by arrow heads, arrows indicate infection threads (scale bar 50 μm; ep=epidermis; C4=cortical cell layer 4; C5=cortical cell layer 5; ed=endodermis; pc=pericycle). The images shown in FIGS. 10A-10E are representative images from multiple replicates.

FIGS. 11A-11F show the CE region is required for rhizobium-induced NIN expression in the pericycle 48 hours post inoculation with S. meliloti RCR2011. FIGS. 11A-11B show M. truncatula A17 WT roots transformed with ProNIN_{5kb}:GUS (FIG. 11A) and ProNIN_{CE-5kb}:GUS (FIG. 11B). In FIGS. 11A-11B, GUS expression is in the epidermis, the endodermis, and the pericycle, and is indicated by arrowheads (lower GUS expression is also in some cortical cells; not indicated by arrowheads). FIGS. 11C-11D show M. truncatula daphne-like mutant roots transformed with ProNIN_{5kb}:GUS (FIG. 11C) and ProNIN_{CE-5kb}:GUS (FIG. 11D). In FIG. 11C, GUS expression is in the epidermis and the outer cortex, and is indicated by arrowheads. In FIG. 11D, GUS expression is in the epidermis, the outer cortex, and the pericycle (weaker expression in the pericycle), and is indicated by arrowheads. FIGS. 11E-11F show M. truncatula nin-1 mutant roots transformed with ProNIN_{5kb}:GUS (FIG. 11E) and ProNIN_{CE-5kb}:GUS (FIG. 11F). In FIGS. 11E-11F, GUS expression is in the epidermis and the outer cortex, and is indicated by arrowheads. In FIGS. 11A-11F, scale bar 50 μm; ep=epidermis; C4=cortical cell layer 4; C5=cortical cell layer 5; ed=endodermis; pc=pericycle). The images shown in FIGS. 11A-11F are representative images from multiple replicates.

FIGS. 12A and 12B show CRE1 and RR1 RNA localization in non-inoculated M. truncatula A17 WT roots. FIG. 12A shows RNA in situ localization of CRE1 in non-inoculated roots, where hybridization signals are depicted as dark dots and indicated by arrow heads (scale bar 50 μm; ep=epidermis; C4=cortical cell layer 4; C5=cortical cell layer 5; ed=endodermis; pc=pericycle; vb=vascular bundle). FIG. 12B shows RNA in situ localization of RR1 in non-inoculated roots, where hybridization signals are depicted as dark dots and indicated by arrow heads (scale bar 50 μm; ep=epidermis; C4=cortical cell layer 4; C5=cortical cell layer 5; ed=endodermis; pc=pericycle; vb=vascular bundle). The images shown in FIGS. 12A-12B are representative images from multiple replicates.

FIG. 13 shows a model for NIN function during nodule primordium initiation. The bacterial colony is shown as a grey dot in the curl of the root hair, whereas the infection thread is shown as a light grey line in the shaft of the root hair (root hair is depicted as vertical protrusion from the epidermis of the cell). The annotations “−2.2 Kb” and “−5 Kb” denote portions of the NIN 5′-upstream region (ep=epidermis; C4=cortical cell layer 4; C5=cortical cell layer 5; ed=endodermis; pc=pericycle).

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

### Genetically Altered Plants and Seeds

An aspect of the disclosure includes a genetically altered plant, wherein the plant or a part thereof includes one or more genetic alterations that increase activity of a NODULE INCEPTION (NIN) protein or a NIN-like protein (NLP protein) in response to cytokinin signaling as compared to a wild type (WT) plant without the one or more genetic alterations, and wherein the plant or the part thereof includes a nucleic acid encoding the NIN protein or the NLP protein. An additional embodiment of this aspect includes the one or more genetic alterations being addition of one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, or twenty-four or more cytokinin response elements operably linked to the nucleic acid encoding the NIN protein or the NLP protein. Still another embodiment of this aspect includes the one or more genetic alterations being eight or more, sixteen or more, or twenty-four or more cytokinin response elements operably linked to the nucleic acid encoding the NIN protein or the NLP protein. Yet another embodiment of this aspect includes at least one of the cytokinin response elements being a B-type cytokinin signaling RESPONSE REGULATOR (RR) binding site. A further embodiment of this aspect includes at least one of the B-type cytokinin signaling RR binding sites having the sequence of SEQ ID NO:613 or SEQ ID NO:614. Yet another embodiment of this aspect includes at least one of the B-type cytokinin signaling RR binding sites having the sequence of SEQ ID NO:679, SEQ ID NO:680, SEQ ID NO:681, SEQ ID NO:682, SEQ ID NO:683, SEQ ID NO:684, SEQ ID NO:685, or SEQ ID NO:686. Still another embodiment of this aspect includes at least one of the B-type cytokinin signaling RR binding sites having the sequence selected from the group of SEQ ID NO:551, SEQ ID NO:552, SEQ ID NO:553, SEQ ID NO:554, SEQ ID NO:555, SEQ ID NO:556, SEQ ID NO:557, SEQ ID NO:558, SEQ ID NO:559, SEQ ID NO:560, SEQ ID NO:561, SEQ ID NO:562, SEQ ID NO:563, SEQ ID NO:564, SEQ ID NO:565, SEQ ID NO:566, SEQ ID NO:567, SEQ ID NO:568, SEQ ID NO:569, SEQ ID NO:570, SEQ ID NO:571, SEQ ID NO:572, SEQ ID NO:573, SEQ ID NO:574, SEQ ID NO:575, SEQ ID NO:576, SEQ ID NO:577, SEQ ID NO:578, SEQ ID NO:579, SEQ ID NO:580, SEQ ID NO:581, SEQ ID NO:582, SEQ ID NO:583, SEQ ID NO:584, SEQ ID NO:585, SEQ ID NO:586, SEQ ID NO:587, SEQ ID NO:588, SEQ ID NO:589, SEQ ID NO:590, SEQ ID NO:591, SEQ ID NO:592, SEQ ID NO:593, SEQ ID NO:594, SEQ ID NO:595, SEQ ID NO:596, SEQ ID NO:597, SEQ ID NO:598, SEQ ID NO:599, SEQ ID NO:600, SEQ ID NO:601, SEQ ID NO:602, SEQ ID NO:603, SEQ ID NO:604, SEQ ID NO:605, SEQ ID NO:606, SEQ ID NO:607, SEQ ID NO:608, SEQ ID NO:609, SEQ ID NO:610, SEQ ID NO:611, SEQ ID NO:612, SEQ ID NO:615, SEQ ID NO:616, SEQ ID NO:617, SEQ ID NO:618, SEQ ID NO:619, SEQ ID NO:620, SEQ ID NO:621, SEQ ID NO:622, SEQ ID NO:623, SEQ ID NO:624, SEQ ID NO:625, SEQ ID NO:626, SEQ ID NO:679, SEQ ID NO:680, SEQ ID NO:681, SEQ ID NO:682, SEQ ID NO:683, SEQ ID NO:684, SEQ ID NO:685, or SEQ ID NO:686. An additional embodiment of this aspect includes the cytokinin response elements being oriented in tandem or being oriented inversely.

In yet another embodiment, which may be combined with any of the preceding embodiments, the cytokinin response elements are within 100 nucleotides, within 95 nucleotides, within 90 nucleotides, within 85 nucleotides, within 80 nucleotides, within 75 nucleotides, within 70 nucleotides, within 65 nucleotides, within 60 nucleotides, within 59 nucleotides, within 58 nucleotides, within 57 nucleotides, within 56 nucleotides, within 55 nucleotides, within 54 nucleotides, within 53 nucleotides, within 52 nucleotides, within 51 nucleotides, within 50 nucleotides, within 49 nucleotides, within 48 nucleotides, within 47 nucleotides, within 46 nucleotides, within 45 nucleotides, within 44 nucleotides, within 43 nucleotides, within 42 nucleotides, within 41 nucleotides, within 40 nucleotides, within 39 nucleotides, within 38 nucleotides, within 37 nucleotides, within 36 nucleotides, within 35 nucleotides, within 34 nucleotides, within 33 nucleotides, within 32 nucleotides, within 31 nucleotides, within 30 nucleotides, within 29 nucleotides, within 28 nucleotides, within 27 nucleotides, within 26 nucleotides, within 25 nucleotides, within 24 nucleotides, within 23 nucleotides, within 22 nucleotides, within 21 nucleotides, within 20 nucleotides, within 19 nucleotides, within 18 nucleotides, within 17 nucleotides, within 16 nucleotides, within 15 nucleotides, within 14 nucleotides, within 13 nucleotides, within 12 nucleotides, within 11 nucleotides, within 10 nucleotides, within 9 nucleotides, within 8 nucleotides, within 7 nucleotides, within 6 nucleotides, within 5 nucleotides, within 4 nucleotides, within 3 nucleotides, within 2 nucleotides, or within 1 nucleotide of each other. In an additional embodiment of this aspect, the cytokinin response elements are within 11 nucleotides, within 10 nucleotides, within 9 nucleotides, within 8 nucleotides, within 7 nucleotides, within 6 nucleotides, within 5 nucleotides, within 4 nucleotides, within 3 nucleotides, within 2 nucleotides, or within 1 nucleotide of each other. In yet another embodiment, which may be combined with any of the preceding embodiments, the nucleic acid encoding the NIN protein or the NLP protein is operably linked to a promoter that is operably linked to the cytokinin response elements. In an additional embodiment of this aspect, the promoter and the cytokinin response elements are within 110,000 nucleotides, within 109,000 nucleotides, within 108,000 nucleotides, within 107,000 nucleotides, within 106,000 nucleotides, within 105,000 nucleotides, within 104,000 nucleotides, within 103,000 nucleotides, within 102,000 nucleotides, within 101,000 nucleotides, within 100,000 nucleotides, within 99,000 nucleotides, within 98,000 nucleotides, within 97,000 nucleotides, within 96,000 nucleotides, within 95,000 nucleotides, within 94,000 nucleotides, within 93,000 nucleotides, within 92,000 nucleotides, within 91,000 nucleotides, within 90,000 nucleotides, within 89,000 nucleotides, within 88,000 nucleotides, within 87,000 nucleotides, within 86,000 nucleotides, within 85,000 nucleotides, within 84,000 nucleotides, within 83,000 nucleotides, within 82,000 nucleotides, within 81,000 nucleotides, within 80,000 nucleotides, within 79,000 nucleotides, within 78,000 nucleotides, within 77,000 nucleotides, within 76,000 nucleotides, within 75,000 nucleotides, within 74,000 nucleotides, within 73,000 nucleotides, within 72,000 nucleotides, within 71,000 nucleotides, within 70,000 nucleotides, within 69,000 nucleotides, within 68,000 nucleotides, within 67,000 nucleotides, within 66,000 nucleotides, within 65,000 nucleotides, within 64,000 nucleotides, within 63,000 nucleotides, within 62,000 nucleotides, within 61,000 nucleotides, within 60,000 nucleotides, within 59,000 nucleotides, within 58,000 nucleotides, within 57,000 nucleotides, within 56,000 nucleotides, within 55,000 nucleotides, within 54,000 nucleotides, within 53,000 nucleotides, within 52,000 nucleotides, within 51,000 nucleotides, within 50,000 nucleotides, within 49,000 nucleotides, within 48,000 nucleotides, within 47,000 nucleotides, within 46,000 nucleotides, within 45,000 nucleotides, within 44,000 nucleotides, within 43,000 nucleotides, within 42,000 nucleotides, within 41,000 nucleotides, within 40,000 nucleotides, within 39,000 nucleotides, within 38,000 nucleotides, within 37,000 nucleotides, within 36,000 nucleotides, within 35,000 nucleotides, within 34,000 nucleotides, within 33,000 nucleotides, within 32,000 nucleotides, within 31,000 nucleotides, within 30,000 nucleotides, within 29,000 nucleotides, within 28,000 nucleotides, within 27,000 nucleotides, within 26,000 nucleotides, within 25,000 nucleotides, within 24,000 nucleotides, within 23,000 nucleotides, within 22,000 nucleotides, within 21,000 nucleotides, within 20,000 nucleotides, within 19,000 nucleotides, within 18,000 nucleotides, within 17,000 nucleotides, within 16,000 nucleotides, within 15,000 nucleotides, within 14,000 nucleotides, within 13,000 nucleotides, within 12,000 nucleotides, within 11,000 nucleotides, within 10,000 nucleotides, within 9,000 nucleotides, within 8,000 nucleotides, within 7,000 nucleotides, within 6,000 nucleotides, within 5,000 nucleotides, within 4,000 nucleotides, within 3,000 nucleotides, within 2,000 nucleotides, within 1,000 nucleotides, within 900 nucleotides, within 800 nucleotides, within 700 nucleotides, within 600 nucleotides, within 500 nucleotides, within 400 nucleotides, within 300 nucleotides, within 200 nucleotides, or within 100 nucleotides of each other. Yet another embodiment of this aspect includes the cytokinin response elements being located upstream of the nucleic acid encoding the NIN protein or the NLP protein. Still another embodiment of this aspect includes the cytokinin response elements being placed between the end of the coding sequence of a 5′-upstream located gene and the transcriptional or translational start site of the nucleic acid encoding the NIN protein or the NLP protein. A further embodiment of this aspect includes the cytokinin response elements being located within the nucleic acid encoding the NIN protein or the NLP protein (i.e., within the transcribed gene sequence). An additional embodiment of this aspect includes the cytokinin response elements being located within one or more introns of the nucleic acid encoding the NIN protein or the NLP protein.

Still another embodiment of this aspect that can be combined with any of the preceding embodiments includes the nucleic acid encoding a NIN/NLP1 orthogroup protein. An additional embodiment of this aspect includes the NIN/NLP1 orthogroup protein having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to an amino acid sequence selected from the group of SEQ ID NO:1, SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22; SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:108, SEQ ID NO:109, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:687, SEQ ID NO:688, SEQ ID NO:689, SEQ ID NO:690, SEQ ID NO:691, SEQ ID NO:692, or SEQ ID NO:693. A further embodiment of this aspect includes the NIN/NLP1 orthogroup protein being a NIN protein and having at least at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to an amino acid sequence selected from the group of SEQ ID NO:22 (i.e., CsaNIN; Cannabis sativa), SEQ ID NO:78 (i.e., HluNIN; Humulus lupulus), SEQ ID NO:89 (i.e., LjNIN; Lotus japonicus), SEQ ID NO:108 (i.e., MtNIN; Medicago truncatula); SEQ ID NO:136 (i.e., PanNIN; Parasponia andersonii), SEQ ID NO:139 (i.e., PriNIN; Parasponia rigida), SEQ ID NO:142 (i.e., PruNIN; Parasponia rugosa), SEQ ID NO:185 (i.e., TleNIN; Trema levigata; truncated version of NIN), SEQ ID NO:187 (i.e., TorNIN; Trema orientalis; truncated version of NIN), SEQ ID NO:190 (i.e., TtoNIN; Trema tomentosa; truncated version of NIN), SEQ ID NO:236 (i.e., ZjuNIN; Ziziphus jujuba), SEQ ID NO:687 (i.e., AglNIN; Alnus glutinosa), SEQ ID NO:688 (i.e., CglNIN; Casuarina glauca), SEQ ID NO:689 (i.e., DglNIN.1; Datisca glomerata), SEQ ID NO:690 (i.e., DglNIN.2; Datisca glomerata), SEQ ID NO:691 (i.e., DtrNIN; Discaria trinervis), SEQ ID NO:692 (i.e., DdrNIN; Dryas drummondii), or SEQ ID NO:693 (i.e., PtrNIN; Purshia tridentata). Another embodiment of this aspect includes the NIN/NLP1 orthogroup protein being a NIN protein and having at least at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to an amino acid sequence selected from the group of SEQ ID NO:89 (i.e., LjNIN; Lotus japonicus) or SEQ ID NO:108 (i.e., MtNIN; Medicago truncatula).

Yet another embodiment of this aspect that can be combined with any of the preceding embodiments includes the nucleic acid encoding a NLP2-3 orthogroup protein, a NLP4 orthogroup protein, or a basal NIN/NLP orthogroup protein. An additional embodiment of this aspect includes the NLP2-3 orthogroup protein having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to an amino acid sequence selected from the group of SEQ ID NO:237, SEQ ID NO:238, SEQ ID NO:239, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, SEQ ID NO:282, SEQ ID NO:283, SEQ ID NO:284, SEQ ID NO:285, SEQ ID NO:286, SEQ ID NO:287, SEQ ID NO:288, SEQ ID NO:289, SEQ ID NO:290, SEQ ID NO:291, SEQ ID NO:292, SEQ ID NO:293, SEQ ID NO:294, SEQ ID NO:295, SEQ ID NO:296, SEQ ID NO:297, SEQ ID NO:298, SEQ ID NO:299, SEQ ID NO:300, SEQ ID NO:301, SEQ ID NO:302, SEQ ID NO:303, SEQ ID NO:304, SEQ ID NO:305, SEQ ID NO:306, SEQ ID NO:307, SEQ ID NO:308, SEQ ID NO:309, SEQ ID NO:310, SEQ ID NO:311, SEQ ID NO:312, SEQ ID NO:313, SEQ ID NO:314, SEQ ID NO:315, SEQ ID NO:316, SEQ ID NO:317, SEQ ID NO:318, SEQ ID NO:319, SEQ ID NO:320, SEQ ID NO:321, SEQ ID NO:322, SEQ ID NO:323, SEQ ID NO:324, SEQ ID NO:325, SEQ ID NO:326, SEQ ID NO:327, SEQ ID NO:328, SEQ ID NO:329, SEQ ID NO:332, SEQ ID NO:333, SEQ ID NO:334, SEQ ID NO:335, SEQ ID NO:336, SEQ ID NO:337, SEQ ID NO:338, SEQ ID NO:339, SEQ ID NO:340, SEQ ID NO:341, SEQ ID NO:342, SEQ ID NO:343, SEQ ID NO:344, SEQ ID NO:345, SEQ ID NO:346, SEQ ID NO:347, SEQ ID NO:348, SEQ ID NO:349, SEQ ID NO:350, SEQ ID NO:351, SEQ ID NO:352, SEQ ID NO:353, SEQ ID NO:354, SEQ ID NO:355, SEQ ID NO:356, SEQ ID NO:357, SEQ ID NO:358, SEQ ID NO:359, SEQ ID NO:360, SEQ ID NO:361, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:371, SEQ ID NO:372, SEQ ID NO:373, SEQ ID NO:374, SEQ ID NO:375, SEQ ID NO:376, and SEQ ID NO:377. Yet another embodiment of this aspect includes the NLP4 orthogroup protein having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to an amino acid sequence selected from the group of SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:391, SEQ ID NO:392, SEQ ID NO:393, SEQ ID NO:394, SEQ ID NO:395, SEQ ID NO:396, SEQ ID NO:397, SEQ ID NO:398, SEQ ID NO:399, SEQ ID NO:400, SEQ ID NO:401, SEQ ID NO:402, SEQ ID NO:403, SEQ ID NO:404, SEQ ID NO:405, SEQ ID NO:406, SEQ ID NO:408, SEQ ID NO:409, SEQ ID NO:410, SEQ ID NO:411, SEQ ID NO:412, SEQ ID NO:413, SEQ ID NO:414, SEQ ID NO:415, SEQ ID NO:417, SEQ ID NO:418, SEQ ID NO:419, SEQ ID NO:420, SEQ ID NO:421, SEQ ID NO:422, SEQ ID NO:423, SEQ ID NO:424, SEQ ID NO:425, SEQ ID NO:426, SEQ ID NO:427, SEQ ID NO:428, SEQ ID NO:429, SEQ ID NO:430, SEQ ID NO:431, SEQ ID NO:432, SEQ ID NO:433, SEQ ID NO:434, SEQ ID NO:435, SEQ ID NO:436, SEQ ID NO:437, SEQ ID NO:438, SEQ ID NO:439, SEQ ID NO:440, SEQ ID NO:441, SEQ ID NO:442, SEQ ID NO:443, SEQ ID NO:444, SEQ ID NO:445, SEQ ID NO:446, SEQ ID NO:447, SEQ ID NO:448, SEQ ID NO:449, SEQ ID NO:450, SEQ ID NO:451, SEQ ID NO:452, SEQ ID NO:453, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, SEQ ID NO:484, SEQ ID NO:485, SEQ ID NO:486, SEQ ID NO:487, SEQ ID NO:488, SEQ ID NO:489, SEQ ID NO:490, SEQ ID NO:491, SEQ ID NO:492, SEQ ID NO:493, SEQ ID NO:494, SEQ ID NO:495, SEQ ID NO:496, SEQ ID NO:497, SEQ ID NO:498, SEQ ID NO:499, SEQ ID NO:500, SEQ ID NO:501, SEQ ID NO:502, SEQ ID NO:504, SEQ ID NO:505, SEQ ID NO:506, SEQ ID NO:507, SEQ ID NO:508, SEQ ID NO:509, SEQ ID NO:510, SEQ ID NO:511, SEQ ID NO:512, SEQ ID NO:513, SEQ ID NO:514, SEQ ID NO:515, SEQ ID NO:516, SEQ ID NO:517, SEQ ID NO:518, SEQ ID NO:519, SEQ ID NO:520, SEQ ID NO:521, SEQ ID NO:522, SEQ ID NO:523, and SEQ ID NO:524. A further embodiment of this aspect includes the basal NIN/NLP orthogroup protein having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to an amino acid sequence selected from the group of SEQ ID NO:637, SEQ ID NO:638, SEQ ID NO:639, SEQ ID NO:640, SEQ ID NO:641, SEQ ID NO:642, SEQ ID NO:643, SEQ ID NO:644, SEQ ID NO:645, SEQ ID NO:646, SEQ ID NO:647, SEQ ID NO:648, SEQ ID NO:649, SEQ ID NO:650, SEQ ID NO:651, SEQ ID NO:652, SEQ ID NO:653, SEQ ID NO:654, SEQ ID NO:655, SEQ ID NO:656, SEQ ID NO:657, SEQ ID NO:658, SEQ ID NO:659, SEQ ID NO:660, SEQ ID NO:661, or SEQ ID NO:662.

In yet another embodiment, which may be combined with any of the preceding embodiments, the nucleic acid encoding the NIN protein or the NLP protein is endogenous. Still another embodiment of this aspect that can be combined with any of the preceding embodiments includes the nucleic acid encoding the NIN protein or the NLP protein being heterologous. Yet another embodiment of this present aspect that can be combined with any of the preceding aspects that has the nucleic acid encoding the NIN protein or the NLP protein operably linked to a promoter includes the promoter being endogenous. Still another embodiment of this aspect that can be combined with any of the preceding aspects that has the nucleic acid encoding the NIN protein or the NLP protein operably linked to a promoter includes the promoter being heterologous.

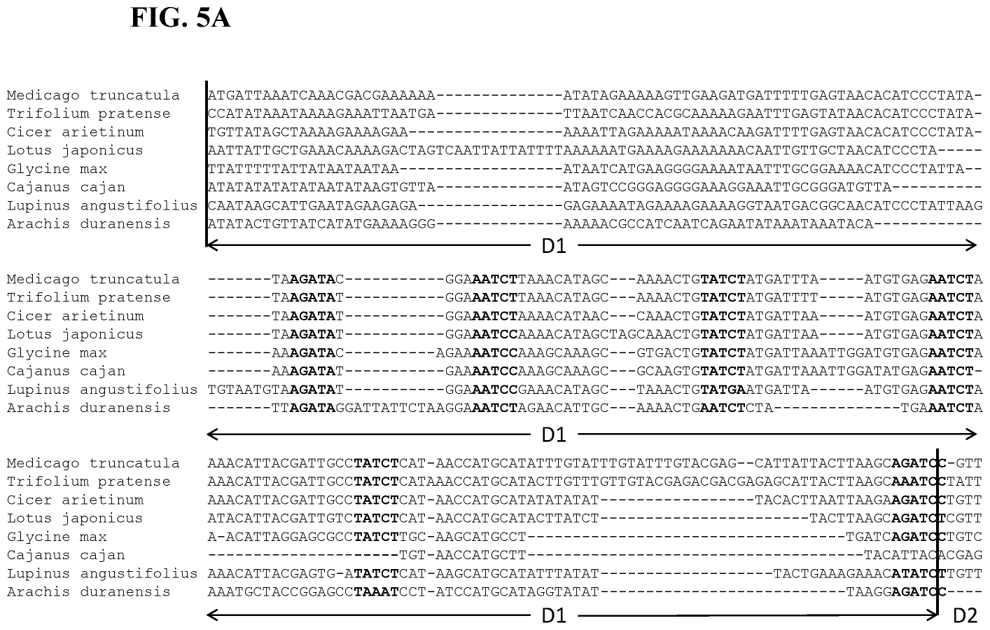

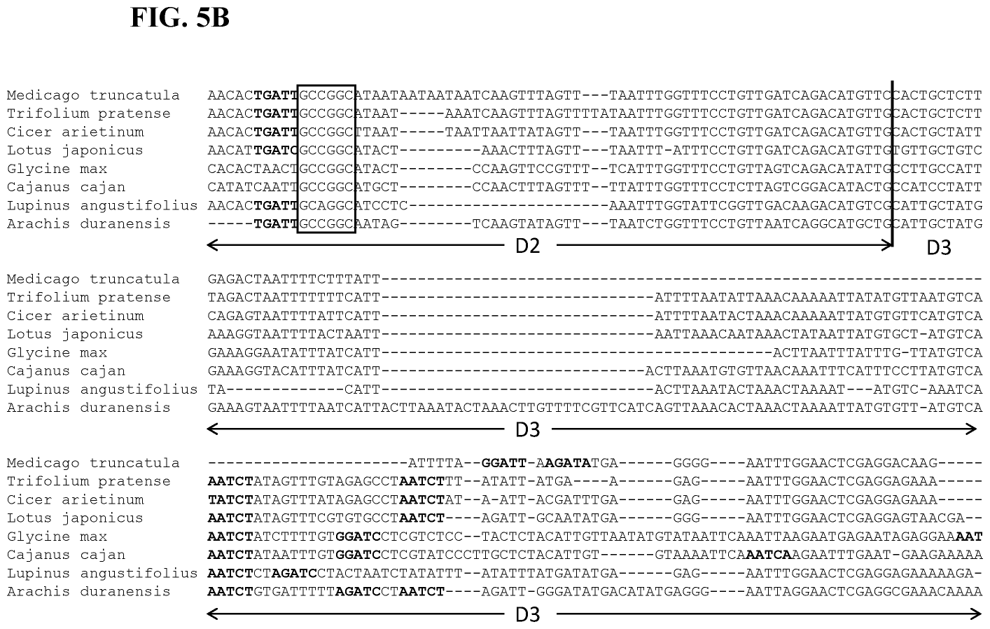

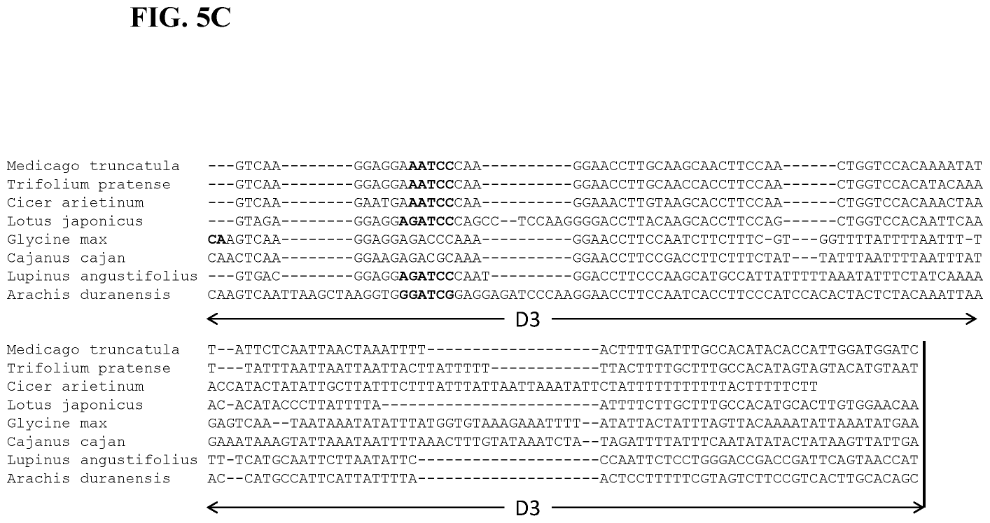

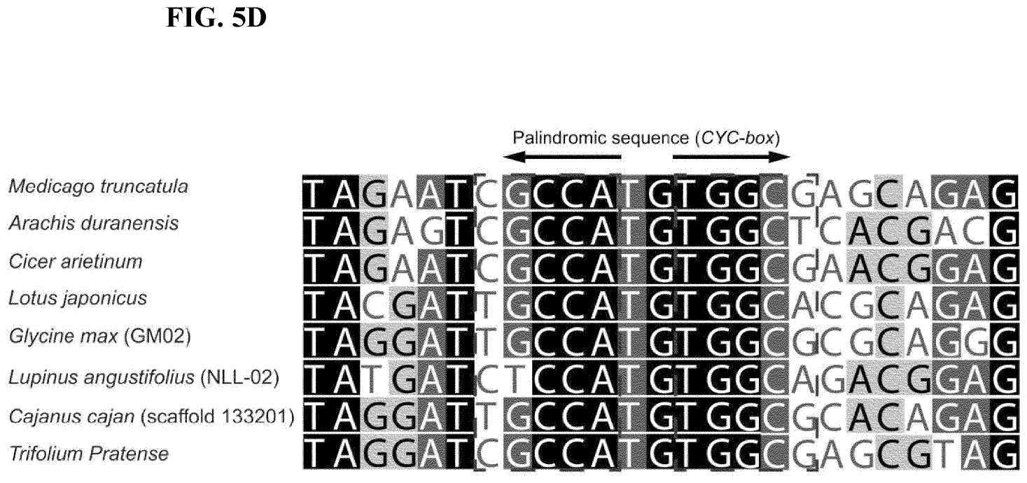

In still another embodiment, which may be combined with any of the preceding embodiments, cytokinin signaling or induction of the cytokinin signaling pathway in a root pericycle cell layer, an endodermis cell layer (i.e., endodermal cell layer), cortex cell layers (i.e., cortical cell layers), and/or an epidermis cell layer (i.e., epidermal cell layer) induces nodule organogenesis. An additional embodiment of this aspect includes the cytokinin signaling pathways being induced by cytokinin analogs that are exogenously applied or secreted by microbes. Still another embodiment of this aspect that can be combined with any of the preceding embodiments further includes one or more CYCLOPS response elements operably linked to the nucleic acid. CYCLOPS response elements of the present disclosure may be a full CYCLOPS response element or an essential CYCLOPS response element (CYC-box) as shown in FIG. 5D. An additional embodiment of this aspect includes CYCLOPS expression in a root epidermis cell layer (i.e., epidermal cell layer) inducing rhizobium infection.

In a further embodiment, which may be combined with any of the preceding embodiments, the genetically altered plant is a monocot. An additional embodiment of this aspect includes the genetically altered plant being selected from the group of corn (e.g., maize, Zea mays), rice (e.g., indica rice, japonica rice, aromatic rice, glutinous rice, Oryza sativa, Oryza glaberrima), wild rice (e.g., Zizania spp., Porteresia spp.), wheat (e.g., common wheat, spelt, durum, einkom, emmer, kamut, Triticum aestivum, Triticum spelta, Triticum durum, Triticum urartu, Triticum monococcum, Triticum turanicum, Triticum spp.), barley (e.g., Hordeum vulgare), sorghum (e.g., Sorghum bicolor), millet (e.g., finger millet, fonio millet, foxtail millet, pearl millet, barnyard millets, Eleusine coracana, Panicum sumatrense, Panicum milaceum, Setaria italica, Pennisetum glaucum, Digitaria spp., Echinocloa spp.), teff (e.g., Eragrostis tef), oat (e.g., Avena sativa), triticale (e.g., X Triticosecale Wittmack, Triticosecale schlanstedtense Triticosecale neoblaringhemii A. Camus, Triticosecale neoblaringhemii A. Camus), rye (e.g., Secale cereale, Secale cereanum), or sugar cane (e.g., Saccharum officinarum, Saccharum spp.). Still another embodiment of this aspect that can be combined with any of the preceding embodiments further includes the genetically altered plant being selected from the group of apple (e.g., Malus pumila, Malus x domestica, Pyrus malus), pear (e.g., Pyrus communis, Pyrus x bretschneideri, Pyrus pyrifolia, Pyrus sinkiangensis, Pyrus pashia, Pyrus spp.), plum (e.g., Mirabelle, greengage, damson, Prunus domestica, Prunus salicina, Prunus mume), apricot (e.g., Prunus armeniaca, Prunus brigantine, Prunus mandshurica), peach (e.g., Prunus persica), almond (e.g., Prunus dulcis, Prunus amygdalus), walnut (e.g., Persian walnut, English walnut, black walnut, Juglans regia, Juglans nigra, Juglans cinerea, Juglans californica), cherry (e.g., Prunus avium, Prunus cerasus, Prunus yedoensis var. nudiflora), strawberry (e.g., Fragaria x ananassa, Fragaria chiloensis, Fragaria virginiana, Fragaria vesca), raspberry (e.g., European red raspberry, black raspberry, Rubus idaeus L., Rubus occidentalis, Rubus strigosus), blackberry (e.g., evergreen blackberry, Himalayan blackberry, Rubus fruticosus, Rubus ursinus, Rubus laciniatus, Rubus argutus, Rubus armeniacus, Rubus plicatus, Rubus ulmifolius, Rubus allegheniensis, Rubus subgenus Eubatus sect. Moriferi & Ursini), red currant (e.g., white currant, Ribes rubrum), black currant (e.g., cassis, Ribes nigrum), gooseberry (e.g., Ribes uva-crispa, Ribes grossulari, Ribes hirtellum), melon (e.g., watermelon, winter melon, casabas, cantaloupe, honeydew, muskmelon, Citrullus lanatus, Benincasa hispida, Cucumis melo, Cucumis melo cantalupensis, Cucumis melo inodorus, Cucumis melo reticulatus), cucumber (e.g., slicing cucumbers, pickling cucumbers, English cucumber, Cucumis sativus), pumpkin (e.g., Cucurbita pepo, Cucurbita maxima), squash (e.g., gourd, Cucurbita argyrosperma, Cucurbita ficifolia, Cucurbita maxima, Cucurbita moschata), grape (e.g., Vitis vinifera, Vitis amurensis, Vitis labrusca, Vitis mustangensis, Vitis riparia, Vitis rotundifolia), hemp (e.g., cannabis, Cannabis sativa), hops (e.g., Humulus lupulus), birch (e.g., Betula spp.), beech (e.g., Fagus sylvatica, Fagus grandifolia, Fagus spp.), jujube (e.g., red date, Ziziphus jujube), cassava (e.g., manioc, yucca, Manihot esculenta), poplar (e.g., hybrid poplar, Populus trichocarpa, Populus tremula, Populus alba, Populus spp.), chestnut (e.g., Castanea mollissima, Castanea crenata, Castanea dentata, Castanea spp.), swamp oak (e.g., Casuarina glauca), rose gum (e.g., Eucalyptus grandis), oak (e.g., cork oak, Quercus suber, Quercus spp.), citrus (e.g., lemon, lime, orange, grapefruit, pomelo, citron, trifoliate orange, bergamot orange, bitter orange, blood orange, satsuma, clementine, mandarin, yuzu, finger lime, kaffir lime, kumquat, Citrus clementina, Citrus sinensis, Citrus trifoliata, Citrus japonica, Citrus maxima, Citrus australasica, Citrus reticulata, Citus aurantifolia, Citrus hystrix, Citrus x paradisi, Citrus x clementina, Citrus spp.), potato (e.g., russet potatoes, yellow potatoes, red potatoes, Solanum tuberosum), tomato (e.g., Solanum lycopersicum), sweet potato (e.g., Ipomoea batatas), yam (e.g., Diascorea spp., Oxalis tuberosa), Arabidopsis (e.g., Arabidopsis thaliana), Trema spp. (e.g., Trema cannabina, Trema cubense, Trema discolor, Trema domingensis, Trema integerrima, Trema lamarckiana, Trema micrantha, Trema orientalis, Trema philippinensis, Trema strigilosa, Trema tomentosa, Trema levigata), and Jatropha spp. (e.g., Jatropha curcas). In yet another embodiment, which may be combined with any of the preceding embodiments, the WT plant is not a legume, does not form nodules for symbiosis with nitrogen fixing bacteria, or both is not a legume and does not form nodules for symbiosis with nitrogen fixing bacteria.

Still another embodiment of this aspect that can be combined with any of the preceding embodiments includes a genetically altered plant part of the genetically altered plant of any one of the preceding embodiments, wherein the plant part is a leaf, a stem, a root, a tuber, a flower, a seed, a kernel, a grain, a fruit, a cell, or a portion thereof and the genetically altered plant part includes the one or more genetic alterations. An additional embodiment of this aspect includes the plant part being a fruit, a tuber, a kernel, or a grain. Yet another embodiment of this aspect that can be combined with any of the preceding embodiments includes a genetically altered pollen grain or a genetically altered ovule of the plant of any one of the preceding embodiments, wherein the genetically altered pollen grain or the genetically altered ovule includes the one or more genetic alterations. A further embodiment of this aspect that can be combined with any of the preceding embodiments includes a genetically altered protoplast produced from the genetically altered plant of any of the preceding embodiments, wherein the genetically altered protoplast includes the one or more genetic alterations. An additional embodiment of this aspect that can be combined with any of the preceding embodiments includes a genetically altered tissue culture produced from protoplasts or cells from the genetically altered plant of any one of the preceding embodiments, wherein the cells or protoplasts are produced from a plant part selected from the group of leaf, leaf mesophyll cell, anther, pistil, stem, petiole, root, root tip, tuber, fruit, seed, kernel, grain, flower, cotyledon, hypocotyl, embryo, or meristematic cell, wherein the genetically altered tissue culture includes the one or more genetic alterations. An additional embodiment of this aspect includes a genetically altered plant regenerated from the genetically altered tissue culture that includes the one or more genetic alterations. Still another embodiment of this aspect that can be combined with any of the preceding embodiments that has a genetically altered plant includes the genetically altered plant having all the physiological and morphological characteristics of the plant of any of the preceding embodiments. Yet another embodiment of this aspect that can be combined with any of the preceding embodiments that has a genetically altered plant includes a genetically altered plant seed produced from the genetically altered plant of any one of the preceding embodiments. A further embodiment of this aspect that can be combined with any of the preceding embodiments that has a genetically altered plant includes the seed of the plant producing a plant having all the physiological and morphological characteristics of the plant of any of the above embodiments.

### Methods of Producing and Cultivating Genetically Altered Plants

An additional aspect of the disclosure includes methods of producing the genetically altered plant of any of the preceding embodiments that has a genetically altered plant, including the steps of: (a) introducing the one or more genetic alterations into a plant cell, tissue, or other explant; (b) regenerating the plant cell, tissue, or other explant into a genetically altered plantlet; and (c) growing the genetically altered plantlet into a genetically altered plant with the one or more genetic alterations that increase activity of the NIN protein or the NLP protein in response to cytokinin signaling as compared to an untransformed WT plant. An additional embodiment of this aspect further includes identifying successful introduction of the one or more genetic alterations by screening or selecting the plant cell, tissue, or other explant prior to step (b); screening or selecting plantlets between step (b) and (c); or screening or selecting plants after step (c). In yet another embodiment, which may be combined with any of the preceding embodiments, transformation is done using a transformation method selected from the group of particle bombardment (i.e., biolistics, gene gun), Agrobacterium-mediated transformation, Rhizobium-mediated transformation, or protoplast transfection or transformation.

Still another embodiment of this aspect that can be combined with any of the preceding embodiments includes genetic alterations being introduced with a vector. An additional embodiment of this aspect includes the vector including a promoter operably linked to a nucleotide encoding a NIN or NLP protein and one or more cytokinin response elements operably linked to the promoter. Yet another embodiment of this aspect includes the promoter and the one or more cytokinin response elements being selected from the group of a NIN gene promoter including a 5′-upstream sequence including a CYCLOPS response element through a transcription start site of the NIN gene operably linked to a 3C region, the NIN gene promoter including a 5′-upstream sequence including the CYCLOPS response element through to the transcription start site of the NIN gene operably linked to a CE region, a minimal promoter operably linked to a CYCLOPS response element operably linked to a CE region, and a minimal promoter operably linked to a CYCLOPS response element operably linked to one or more cytokinin response elements. CYCLOPS response elements of the present disclosure may be a full CYCLOPS response element or an essential CYCLOPS response element (CYC-box) as shown in FIG. 5D. In a further embodiment of this aspect, the vector includes one or more gene editing components that target a nuclear genome sequence operably linked to an endogenous NIN protein or NLP protein. Yet another embodiment of this aspect includes the nuclear genome sequence being edited by the one or more gene editing components to introduce a cis-regulatory element selected from the group of one or more cytokinin response elements, a 3C region, or a CE region. Yet another embodiment of this present aspect that can be combined with any of the preceding aspects that has a vector including one or more gene editing components includes one or more gene editing components being selected from the group of a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (ODN), wherein the ODN targets the nuclear genome sequence; or a vector including a CRISPR/Cas enzyme encoding sequence and a targeting sequence, wherein the targeting sequence targets the nuclear genome sequence.

A further embodiment of this present aspect that can be combined with any of the preceding aspects that has genetic alterations being introduced with a vector includes the NIN gene promoter, 3C region, CE region, CYCLOPS response element, or one or more cytokinin response elements being from a nodulating legume species. An additional embodiment of this aspect includes the nodulating legume species being selected from the group of peanut (e.g., Arachis duranensis, Arachis hypogaea, Arachis ipaensis), pigeon pea (e.g., Cajanus cajan), chickpea (e.g., Cicer arietinum), soybean (e.g., Glycine max, Glycine soja), velvet bean (e.g., Mucuna pruriens), bean (e.g., Phaseolus vulgaris), pea (e.g., Pisum sativum), adzuki bean (e.g., Vigna angularis, Vigna angularis var. angularis), mung bean (e.g., Vigna radiata var. radiata), clover (e.g., Trifolium pratense, Trifolium subterraneum), lupine (e.g., lupin, Lupinus angustifolius), Sesbania spp. (e.g., Sesbania rostrata), Lotus japonicus, and Medicago truncatula. Yet another embodiment of this present aspect that can be combined with any of the preceding aspects that has genetic alterations being introduced with a vector wherein the vector includes 5′-upstream NIN sequence, 3C region, CE region, CYCLOPS response element, or one or more cytokinin response elements from a nodulating legume species includes cytokinin response elements being selected from the group of SEQ ID NO:551, SEQ ID NO:552, SEQ ID NO:553, SEQ ID NO:554, SEQ ID NO:555, SEQ ID NO:556, SEQ ID NO:557, SEQ ID NO:558, SEQ ID NO:559, SEQ ID NO:560, SEQ ID NO:561, SEQ ID NO:562, SEQ ID NO:563, SEQ ID NO:564, SEQ ID NO:565, SEQ ID NO:566, SEQ ID NO:567, SEQ ID NO:568, SEQ ID NO:569, SEQ ID NO:570, SEQ ID NO:571, SEQ ID NO:572, SEQ ID NO:573, SEQ ID NO:574, SEQ ID NO:575, SEQ ID NO:576, SEQ ID NO:577, SEQ ID NO:578, SEQ ID NO:579, SEQ ID NO:580, SEQ ID NO:581, SEQ ID NO:582, SEQ ID NO:583, SEQ ID NO:584, SEQ ID NO:585, SEQ ID NO:586, SEQ ID NO:587, SEQ ID NO:588, SEQ ID NO:589, SEQ ID NO:590, SEQ ID NO:591, SEQ ID NO:592, SEQ ID NO:593, SEQ ID NO:594, SEQ ID NO:595, SEQ ID NO:596, SEQ ID NO:597, SEQ ID NO:598, SEQ ID NO:599, SEQ ID NO:600, SEQ ID NO:601, SEQ ID NO:602, SEQ ID NO:603, SEQ ID NO:604, SEQ ID NO:605, SEQ ID NO:606, SEQ ID NO:607, SEQ ID NO:608, SEQ ID NO:609, SEQ ID NO:610, SEQ ID NO:611, or SEQ ID NO:612. Still another embodiment of this present aspect that can be combined with any of the preceding aspects that has genetic alterations being introduced with a vector includes cytokinin response elements being selected from the group of SEQ ID NO:613, SEQ ID NO:614, SEQ ID NO:615, SEQ ID NO:616, SEQ ID NO:617, SEQ ID NO:618, SEQ ID NO:619, SEQ ID NO:620, SEQ ID NO:621, SEQ ID NO:622, SEQ ID NO:623, SEQ ID NO:624, SEQ ID NO:625, and SEQ ID NO:626. Yet another embodiment of this present aspect that can be combined with any of the preceding aspects that has genetic alterations being introduced with a vector includes one or more cytokinin response elements being from a non-nodulating species. In yet another embodiment of this present aspect that can be combined with any of the preceding aspects that has genetic alterations being introduced with a vector, the one or more cytokinin response elements from a non-nodulating species is SEQ ID NO:613. A further embodiment of this present aspect that can be combined with any of the preceding aspects that has genetic alterations being introduced with a vector includes the one or more cytokinin response elements being from a nodulating non-legume species. An additional embodiment of this present aspect that can be combined with any of the preceding aspects that has genetic alterations being introduced with a vector includes cytokinin response elements being selected from the group of SEQ ID NO:613, SEQ ID NO:614, SEQ ID NO:679, SEQ ID NO:680, SEQ ID NO:681, SEQ ID NO:682, SEQ ID NO:683, SEQ ID NO:684, SEQ ID NO:685, or SEQ ID NO:686.

A further embodiment of this present aspect that can be combined with any of the preceding aspects that has genetic alterations being introduced with a vector includes the NIN or NLP protein being a NIN/NLP1 orthogroup protein and having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to an amino acid sequence selected from the group of SEQ ID NO:1, SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22; SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:108, SEQ ID NO:109, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:192, SEQ ID NO:193, SEQ ID NO:194, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:687, SEQ ID NO:688, SEQ ID NO:689, SEQ ID NO:690, SEQ ID NO:691, SEQ ID NO:692, or SEQ ID NO:693. Yet another embodiment of this present aspect that can be combined with any of the preceding aspects that has genetic alterations being introduced with a vector includes the NIN/NLP1 orthogroup protein being a NIN protein and having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to an amino acid sequence selected from the group of SEQ ID NO:22 (i.e., CsaNIN; Cannabis sativa), SEQ ID NO:78 (i.e., HluNIN; Humulus lupulus), SEQ ID NO:89 (i.e., LjNIN; Lotus japonicus), SEQ ID NO:108 (i.e., MtNIN; Medicago truncatula); SEQ ID NO:136 (i.e., PanNIN; Parasponia andersonii), SEQ ID NO:139 (i.e., PriNIN; Parasponia rigida), SEQ ID NO:142 (i.e., PruNIN; Parasponia rugosa), SEQ ID NO:185 (i.e., TleNIN; Trema levigata; truncated version of NIN), SEQ ID NO:187 (i.e., TorNIN; Trema orientalis; truncated version of NIN), SEQ ID NO:190 (i.e., TtoNIN; Trema tomentosa; truncated version of NIN), SEQ ID NO:236 (i.e., ZjuNIN; Ziziphus jujuba), SEQ ID NO:687 (i.e., AglNIN; Alnus glutinosa), SEQ ID NO:688 (i.e., CglNIN; Casuarina glauca), SEQ ID NO:689 (i.e., DglNIN.1; Datisca glomerata), SEQ ID NO:690 (i.e., DglNIN.2; Datisca glomerata), SEQ ID NO:691 (i.e., DtrNIN; Discaria trinervis), SEQ ID NO:692 (i.e., DdrNIN; Dryas drummondii), or SEQ ID NO:693 (i.e., PtrNIN; Purshia tridentata). Another embodiment of this present aspect that can be combined with any of the preceding aspects that has genetic alterations being introduced with a vector includes the NIN/NLP1 orthogroup protein being a NIN protein and having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to an amino acid sequence selected from the group of SEQ ID NO:89 (i.e., LjNIN; Lotus japonicus) or SEQ ID NO:108 (i.e., MtNIN; Medicago truncatula). Still another embodiment of this present aspect that can be combined with any of the preceding aspects that has genetic alterations being introduced with a vector includes the NIN or NLP protein being a NLP2-3 orthogroup protein and having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to an amino acid sequence selected from the group of SEQ ID NO:237, SEQ ID NO:238, SEQ ID NO:239, SEQ ID NO:241, SEQ ID NO:242, SEQ ID NO:243, SEQ ID NO:244, SEQ ID NO:245, SEQ ID NO:246, SEQ ID NO:247, SEQ ID NO:248, SEQ ID NO:250, SEQ ID NO:251, SEQ ID NO:252, SEQ ID NO:253, SEQ ID NO:254, SEQ ID NO:255, SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, SEQ ID NO:267, SEQ ID NO:268, SEQ ID NO:269, SEQ ID NO:270, SEQ ID NO:271, SEQ ID NO:273, SEQ ID NO:274, SEQ ID NO:275, SEQ ID NO:276, SEQ ID NO:277, SEQ ID NO:278, SEQ ID NO:279, SEQ ID NO:280, SEQ ID NO:281, SEQ ID NO:282, SEQ ID NO:283, SEQ ID NO:284, SEQ ID NO:285, SEQ ID NO:286, SEQ ID NO:287, SEQ ID NO:288, SEQ ID NO:289, SEQ ID NO:290, SEQ ID NO:291, SEQ ID NO:292, SEQ ID NO:293, SEQ ID NO:294, SEQ ID NO:295, SEQ ID NO:296, SEQ ID NO:297, SEQ ID NO:298, SEQ ID NO:299, SEQ ID NO:300, SEQ ID NO:301, SEQ ID NO:302, SEQ ID NO:303, SEQ ID NO:304, SEQ ID NO:305, SEQ ID NO:306, SEQ ID NO:307, SEQ ID NO:308, SEQ ID NO:309, SEQ ID NO:310, SEQ ID NO:311, SEQ ID NO:312, SEQ ID NO:313, SEQ ID NO:314, SEQ ID NO:315, SEQ ID NO:316, SEQ ID NO:317, SEQ ID NO:318, SEQ ID NO:319, SEQ ID NO:320, SEQ ID NO:321, SEQ ID NO:322, SEQ ID NO:323, SEQ ID NO:324, SEQ ID NO:325, SEQ ID NO:326, SEQ ID NO:327, SEQ ID NO:328, SEQ ID NO:329, SEQ ID NO:332, SEQ ID NO:333, SEQ ID NO:334, SEQ ID NO:335, SEQ ID NO:336, SEQ ID NO:337, SEQ ID NO:338, SEQ ID NO:339, SEQ ID NO:340, SEQ ID NO:341, SEQ ID NO:342, SEQ ID NO:343, SEQ ID NO:344, SEQ ID NO:345, SEQ ID NO:346, SEQ ID NO:347, SEQ ID NO:348, SEQ ID NO:349, SEQ ID NO:350, SEQ ID NO:351, SEQ ID NO:352, SEQ ID NO:353, SEQ ID NO:354, SEQ ID NO:355, SEQ ID NO:356, SEQ ID NO:357, SEQ ID NO:358, SEQ ID NO:359, SEQ ID NO:360, SEQ ID NO:361, SEQ ID NO:362, SEQ ID NO:363, SEQ ID NO:364, SEQ ID NO:365, SEQ ID NO:366, SEQ ID NO:367, SEQ ID NO:368, SEQ ID NO:369, SEQ ID NO:371, SEQ ID NO:372, SEQ ID NO:373, SEQ ID NO:374, SEQ ID NO:375, SEQ ID NO:376, and SEQ ID NO:377. A further embodiment of this present aspect that can be combined with any of the preceding aspects that has genetic alterations being introduced with a vector includes the NIN or NLP protein being a NLP4 orthogroup protein and having at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to an amino acid sequence selected from the group of SEQ ID NO:378, SEQ ID NO:379, SEQ ID NO:380, SEQ ID NO:381, SEQ ID NO:382, SEQ ID NO:383, SEQ ID NO:384, SEQ ID NO:385, SEQ ID NO:386, SEQ ID NO:387, SEQ ID NO:388, SEQ ID NO:389, SEQ ID NO:390, SEQ ID NO:391, SEQ ID NO:392, SEQ ID NO:393, SEQ ID NO:394, SEQ ID NO:395, SEQ ID NO:396, SEQ ID NO:397, SEQ ID NO:398, SEQ ID NO:399, SEQ ID NO:400, SEQ ID NO:401, SEQ ID NO:402, SEQ ID NO:403, SEQ ID NO:404, SEQ ID NO:405, SEQ ID NO:406, SEQ ID NO:408, SEQ ID NO:409, SEQ ID NO:410, SEQ ID NO:411, SEQ ID NO:412, SEQ ID NO:413, SEQ ID NO:414, SEQ ID NO:415, SEQ ID NO:417, SEQ ID NO:418, SEQ ID NO:419, SEQ ID NO:420, SEQ ID NO:421, SEQ ID NO:422, SEQ ID NO:423, SEQ ID NO:424, SEQ ID NO:425, SEQ ID NO:426, SEQ ID NO:427, SEQ ID NO:428, SEQ ID NO:429, SEQ ID NO:430, SEQ ID NO:431, SEQ ID NO:432, SEQ ID NO:433, SEQ ID NO:434, SEQ ID NO:435, SEQ ID NO:436, SEQ ID NO:437, SEQ ID NO:438, SEQ ID NO:439, SEQ ID NO:440, SEQ ID NO:441, SEQ ID NO:442, SEQ ID NO:443, SEQ ID NO:444, SEQ ID NO:445, SEQ ID NO:446, SEQ ID NO:447, SEQ ID NO:448, SEQ ID NO:449, SEQ ID NO:450, SEQ ID NO:451, SEQ ID NO:452, SEQ ID NO:453, SEQ ID NO:455, SEQ ID NO:456, SEQ ID NO:457, SEQ ID NO:458, SEQ ID NO:459, SEQ ID NO:460, SEQ ID NO:461, SEQ ID NO:462, SEQ ID NO:463, SEQ ID NO:464, SEQ ID NO:465, SEQ ID NO:466, SEQ ID NO:467, SEQ ID NO:468, SEQ ID NO:469, SEQ ID NO:470, SEQ ID NO:471, SEQ ID NO:472, SEQ ID NO:473, SEQ ID NO:474, SEQ ID NO:475, SEQ ID NO:476, SEQ ID NO:477, SEQ ID NO:478, SEQ ID NO:479, SEQ ID NO:480, SEQ ID NO:481, SEQ ID NO:482, SEQ ID NO:483, SEQ ID NO:484, SEQ ID NO:485, SEQ ID NO:486, SEQ ID NO:487, SEQ ID NO:488, SEQ ID NO:489, SEQ ID NO:490, SEQ ID NO:491, SEQ ID NO:492, SEQ ID NO:493, SEQ ID NO:494, SEQ ID NO:495, SEQ ID NO:496, SEQ ID NO:497, SEQ ID NO:498, SEQ ID NO:499, SEQ ID NO:500, SEQ ID NO:501, SEQ ID NO:502, SEQ ID NO:504, SEQ ID NO:505, SEQ ID NO:506, SEQ ID NO:507, SEQ ID NO:508, SEQ ID NO:509, SEQ ID NO:510, SEQ ID NO:511, SEQ ID NO:512, SEQ ID NO:513, SEQ ID NO:514, SEQ ID NO:515, SEQ ID NO:516, SEQ ID NO:517, SEQ ID NO:518, SEQ ID NO:519, SEQ ID NO:520, SEQ ID NO:521, SEQ ID NO:522, SEQ ID NO:523, and SEQ ID NO: 524. An additional embodiment of this present aspect that can be combined with any of the preceding aspects that has genetic alterations being introduced with a vector includes the NIN or NLP protein being a basal NIN/NLP orthogroup protein and having at least at least 70% sequence identity, at least 71% sequence identity, at least 72% sequence identity, at least 73% sequence identity, at least 74% sequence identity, at least 75% sequence identity, at least 76% sequence identity, at least 77% sequence identity, at least 78% sequence identity, at least 79% sequence identity, at least 80% sequence identity, at least 81% sequence identity, at least 82% sequence identity, at least 83% sequence identity, at least 84% sequence identity, at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to an amino acid sequence selected from the group of SEQ ID NO:637, SEQ ID NO:638, SEQ ID NO:639, SEQ ID NO:640, SEQ ID NO:641, SEQ ID NO:642, SEQ ID NO:643, SEQ ID NO:644, SEQ ID NO:645, SEQ ID NO:646, SEQ ID NO:647, SEQ ID NO:648, SEQ ID NO:649, SEQ ID NO:650, SEQ ID NO:651, SEQ ID NO:652, SEQ ID NO:653, SEQ ID NO:654, SEQ ID NO:655, SEQ ID NO:656, SEQ ID NO:657, SEQ ID NO:658, SEQ ID NO:659, SEQ ID NO:660, SEQ ID NO:661, or SEQ ID NO:662.

A further embodiment of the present aspect, which may be combined with any of the preceding embodiments, includes knocking out an endogenous NIN or NLP gene to generate a nin knockout mutant before step (a) and identifying successful complementation of nin knockout mutant by any one of the constructs including a nucleotide encoding a NIN or NLP protein of the preceding embodiments by screening or selecting the plant cell, tissue, or other explant prior to step (b); screening or selecting plantlets between step (b) and (c); or screening or selecting plants after step (c).

A further aspect of the disclosure includes methods of cultivating the genetically altered plant of any of the preceding embodiments that has a genetically altered plant, including the steps of: planting a genetically altered seedling, a genetically altered plantlet, a genetically altered cutting, a genetically altered tuber, a genetically altered root, or a genetically altered seed in soil to produce the genetically altered plant or grafting the genetically altered seedling, the genetically altered plantlet, or the genetically altered cutting to a root stock or a second plant grown in soil to produce the genetically altered plant; cultivating the plant to produce harvestable seed, harvestable leaves, harvestable roots, harvestable cuttings, harvestable wood, harvestable fruit, harvestable kernels, harvestable tubers, and/or harvestable grain; and harvesting the harvestable seed, harvestable leaves, harvestable roots, harvestable cuttings, harvestable wood, harvestable fruit, harvestable kernels, harvestable tubers, and/or harvestable grain.

### Molecular Biological Methods to Produce Genetically Altered Plants and Plant Cells

One embodiment of the present invention provides a genetically altered plant or plant cell including one or more modified cis-regulatory elements and/or introduced cis-regulatory elements. For example, the present disclosure provides genetically altered plants with the addition of one or more cytokinin response elements operably linked to a nucleic acid encoding the NIN protein or the NLP protein where the one or more cytokinin response elements have been introduced by genetic alteration of the plant, the nucleic acid encoding the NIN protein or the NLP protein have been introduced by genetic alteration of the plant, or both the one or more cytokinin response elements and the nucleic acid encoding the NIN protein or the NLP protein have been introduced by genetic alteration of the plant.

Transformation and generation of genetically altered monocotyledonous and dicotyledonous plant cells is well known in the art. See, e.g., Weising, et al., Ann. Rev. Genet. 22:421-477 (1988); U.S. Pat. No. 5,679,558; Agrobacterium Protocols, ed: Gartland, Humana Press Inc. (1995); Wang, et al. Acta Hort. 461:401-408 (1998), and Broothaerts, et al. Nature 433:629-633 (2005). The choice of method varies with the type of plant to be transformed, the particular application and/or the desired result. The appropriate transformation technique is readily chosen by the skilled practitioner.

Any methodology known in the art to delete, insert or otherwise modify the cellular DNA (e.g., genomic DNA and organelle DNA) can be used in practicing the inventions disclosed herein. As an example, the CRISPR/Cas-9 system and related systems (e.g., TALEN, ZFN, ODN, etc.) may be used to insert a heterologous gene to a targeted site in the genomic DNA or substantially edit an endogenous gene to express the heterologous gene. For example, a disarmed Ti plasmid, containing a genetic construct for deletion or insertion of a target gene, in Agrobacterium tumefaciens can be used to transform a plant cell, and thereafter, a transformed plant can be regenerated from the transformed plant cell using procedures described in the art, for example, in EP 0116718, EP 0270822, PCT publication WO 84/02913 and published European Patent application (“EP”) 0242246. Ti-plasmid vectors each contain the gene between the border sequences, or at least located to the left of the right border sequence, of the T-DNA of the Ti-plasmid. Of course, other types of vectors can be used to transform the plant cell, using procedures such as direct gene transfer (as described, for example in EP 0233247), pollen mediated transformation (as described, for example in EP 0270356, PCT publication WO 85/01856, and U.S. Pat. No. 4,684,611), plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm et al., Bio/Technology (1990) 8, 833-839); Gordon-Kamm et al., The Plant Cell, (1990) 2, 603-618), rice (Shimamoto et al., Nature, (1989) 338, 274-276; Datta et al., Bio/Technology, (1990) 8, 736-740), and the method for transforming monocots generally (PCT publication WO 92/09696). For cotton transformation, the method described in PCT patent publication WO 00/71733 can be used. For soybean transformation, reference is made to methods known in the art, e.g., Hinchee et al. (Bio/Technology, (1988) 6, 915) and Christou et al. (Trends Biotech, (1990) 8, 145) or the method of WO 00/42207. Heterologous genes may be from closely related plant species, distantly related plant species, or basal plants (e.g., Physcomitrella spp.) (Possart et al., The Plant Cell, (2017) 29, 310-330; Frangedakis et al., New Phytol, (2017) 216, 591-604).

Genetically altered plants of the present invention can be used in a conventional plant breeding scheme to produce more genetically altered plants with the same characteristics, or to introduce the genetic alteration(s) in other varieties of the same or related plant species. Seeds, which are obtained from the altered plants, preferably contain the genetic alteration(s) as a stable insert in chromosomal DNA or as modifications to an endogenous gene or promoter. Plants including the genetic alteration(s) in accordance with the invention include plants including, or derived from, root stocks of plants including the genetic alteration(s) of the invention, e.g., fruit trees or ornamental plants. Hence, any non-transgenic grafted plant parts inserted on a transformed plant or plant part are included in the invention.

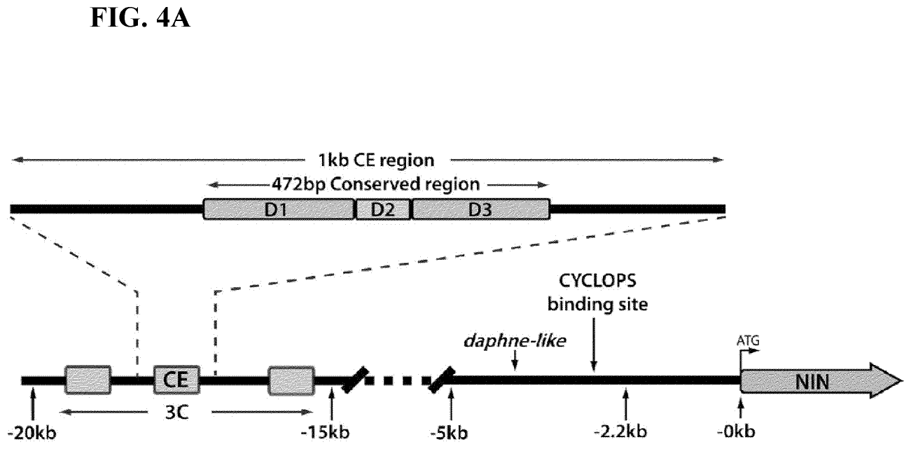

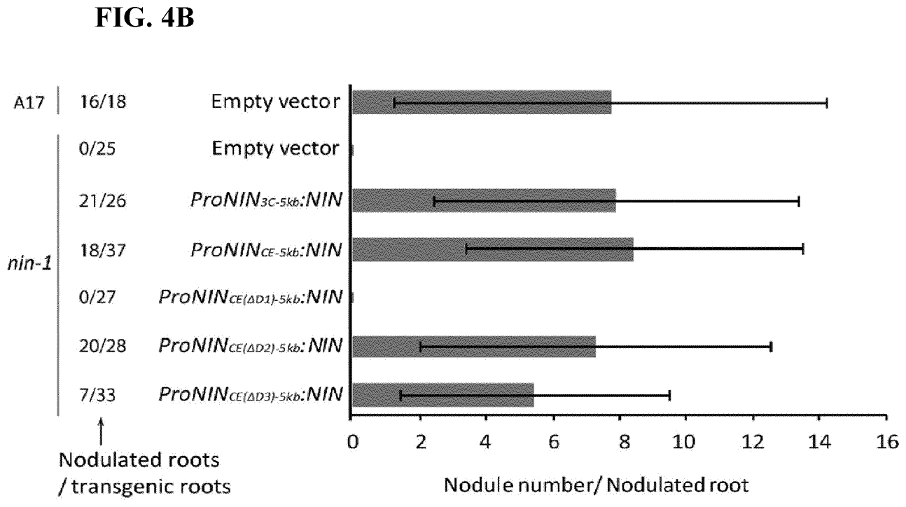

Cis-regulatory elements responsive to cytokinin signaling of the present invention contain B-type cytokinin RESPONSE REGULATOR (RR) binding sites (i.e., cytokinin responsive elements). These cytokinin responsive elements were identified and experimentally characterized in Arabidopsis thaliana in vitro and in vivo studies (Sakai et al., Science, (2001) 294, 1519-1521; Hosoda et al., Plant Cell, (2002) 14, 2015-2029; Imamura et al., Plant Cell Phys, (2003), 22, 122-131, Zhao et al., Nature Letters (2010), 465, 1089-1093), and have also been shown to be conserved in rice (Ross et al., J. Exp. Bot., (2004) 55, 1721-1731). The core conserved element in type-B RR binding sites is the nucleic acid sequence GAT, which is flanked by 5′-(A/G) and 3′-(C/T). The cytokinin responsive elements of the present invention (i.e., cytokinin response element) can be isolated from a plant or synthetic. The cytokinin response elements isolated from a plant can be isolated from 5′-upstream regions of a NIN gene from a nodulating legume species, and can include larger regions (e.g., 3C region, CE region) as shown in FIGS. 4A and 5A-5C. The design of synthetic cytokinin response elements is described in Zürcher et al., Plant Phys, (2013) 161, 1066-1075, which is hereby incorporated by reference.

An introduced cytokinin response element of the present invention may be inserted in host cell DNA so that the inserted cytokinin response element part is upstream (i.e., 5′) of suitable 3′ end transcription regulation signals (e.g., transcript formation and polyadenylation signals). This is preferably accomplished by inserting the cytokinin response element in the plant cell genome (nuclear or chloroplast). In some embodiments, one or more of the introduced cytokinin response elements are stably integrated into the nuclear genome. Stable integration is present when the nucleic acid sequence remains integrated into the nuclear genome and continues to be expressed (e.g., detectable mRNA transcript or protein is produced) throughout subsequent plant generations. Stable integration into and/or editing of the nuclear genome can be accomplished by any known method in the art (e.g., microparticle bombardment, Agrobacterium-mediated transformation, CRISPR/Cas9, electroporation of protoplasts, microinjection, etc.). In some embodiments, a cytokinin response element of the present invention is inserted into host cell DNA along with a NIN or NLP gene. Preferred polyadenylation and transcript formation signals include those of the nopaline synthase gene (Depicker et al., J. Molec Appl Gen, (1982) 1, 561-573), the octopine synthase gene (Gielen et al., EMBO J, (1984) 3:835 845), the SCSV or the Malic enzyme terminators (Schunmann et al., Plant Funct Biol, (2003) 30:453-460), and the T DNA gene 7 (Velten and Schell, Nucleic Acids Res, (1985) 13, 6981 6998), which act as 3′ untranslated DNA sequences in transformed plant cells

Introduced cytokinin response elements are preferably operably linked to a plant-expressible promoter. A ‘plant-expressible promoter’ as used herein refers to a promoter that ensures expression of the genetic alteration(s) of the invention in a plant cell. A plant-expressible promoter can be a 5′-upstream region of a plant gene, such a 5′-upstream region of a NIN gene from a nodulating legume species, which can include 3C regions, CE regions, and/or a CYCLOPS response element. CYCLOPS response elements of the present disclosure may be a full CYCLOPS response element or an essential CYCLOPS response element (CYC-box) as shown in FIG. 5D. Further, a plant-expressible promoter can be a constitutive promoter. In addition, a plant-expressible promoter can be a tissue-specific promoter, e.g., a promoter directing a higher level of expression in some cells or tissues of the plant, e.g., in root pericycle cells.

In preferred embodiments, promoters and other components derived from 5′-upstream regions of NIN genes (i.e., NIN gene promoters) from nodulating legume species will be used. Non-limiting examples include a NIN gene promoter containing a 5′-upstream sequence including a CYCLOPS response element through to the transcription start site of the NIN gene operably linked to a 3C region, a NIN gene promoter containing a 5′-upstream sequence including a CYCLOPS response element through to the transcription start site of the NIN gene operably linked to a CE region, a NIN gene promoter containing a 5′-upstream sequence including a CYCLOPS response element through to the transcription start site of the NIN gene operably linked to one or more cytokinin response elements, a NIN gene promoter operably linked to a 3C region, a NIN gene promoter operably linked to a CE region, and a NIN gene promoter operably linked to one or more cytokinin response elements.

Examples of constitutive promoters that are often used in plant cells are the cauliflower mosaic (CaMV) 35S promoter (KAY et al. Science, 236, 4805, 1987), the minimal CaMV 35S promoter (Benfey & Chua, Science, (1990) 250, 959-966), various other derivatives of the CaMV 35S promoter, the maize ubiquitin promoter (CHRISTENSEN & QUAIL, Transgenic Res, 5, 213-8, 1996), the trefoil promoter (Ljubql, MAEKAWA et al. Mol Plant Microbe Interact. 21, 375-82, 2008), the vein mosaic cassava virus promoter (International Application WO 97/48819), and the Arabidopsis UBQ10 promoter, Norris et al. Plant Mol. Biol. 21, 895-906, 1993). In preferred embodiments, minimal CaMV 35S promoters will be used that contain cytokinin responsive elements. Non-limiting examples include a minimal CaMV 35S promoter operably linked to a CYCLOPS response element operably linked to a CE region, a minimal CaMV 35S promoter operably linked to a CYCLOPS response element operably linked to one or more cytokinin response elements, a minimal CaMV 35S promoter operably linked to a CE region, and a minimal CaMV 35S promoter operably linked to one or more cytokinin response elements.

Additional examples of promoters directing constitutive expression in plants are known in the art and include: the strong constitutive 35S promoters (the “35S promoters”) of the cauliflower mosaic virus (CaMV), e.g., of isolates CM 1841 (Gardner et al., Nucleic Acids Res, (1981) 9, 2871-2887), CabbB S (Franck et al., Cell (1980) 21, 285 294) and CabbB JI (Hull and Howell, Virology, (1987) 86, 482 493); promoters from the ubiquitin family (e.g., the maize ubiquitin promoter of Christensen et al., Plant Mol Biol, (1992) 18, 675-689), the gos2 promoter (de Pater et al., The Plant J (1992) 2, 834-844), the emu promoter (Last et al., Theor Appl Genet, (1990) 81, 581-588), actin promoters such as the promoter described by An et al. (The Plant J, (1996) 10, 107), the rice actin promoter described by Zhang et al. (The Plant Cell, (1991) 3, 1155-1165); promoters of the Cassava vein mosaic virus (WO 97/48819, Verdaguer et al. (Plant Mol Biol, (1998) 37, 1055-1067), the pPLEX series of promoters from Subterranean Clover Stunt Virus (WO 96/06932, particularly the S4 or S7 promoter), an alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the 1′ and 2′ genes, respectively, of the T DNA (Velten et al., EMBO J, (1984) 3, 2723 2730).

Non-limiting examples of tissue-specific promoters include a NFR1 or NFR5/NFP promoter, particularly the Lotus NFR5 promoter (SEQ ID NO: 24) and the Lotus NFR1 promoters (SEQ ID NO: 25) the maize allothioneine promoter (DE FRAMOND et al, FEBS 290, 103-106, 1991 Application EP 452269), the chitinase promoter (SAMAC et al. Plant Physiol 93, 907-914, 1990), the maize ZRP2 promoter (U.S. Pat. No. 5,633,363), the tomato LeExtl promoter (Bucher et al. Plant Physiol. 128, 911-923, 2002), the glutamine synthetase soybean root promoter (HIREL et al. Plant Mol. Biol. 20, 207-218, 1992), the RCC3 promoter (PCT Application WO 2009/016104), the rice antiquitine promoter (PCT Application WO 2007/076115), the LRR receptor kinase promoter (PCT application WO 02/46439), and the Arabidopsis pCO2 promoter (HEIDSTRA et al, Genes Dev. 18, 1964-1969, 2004). These plant promoters can be combined with enhancer elements, they can be combined with minimal promoter elements, or can comprise repeated elements to ensure the expression profile desired.

In some embodiments, genetic elements to increase expression in plant cells can be utilized. For example, an intron at the 5′ end or 3′ end of an introduced gene, or in the coding sequence of the introduced gene, e.g., the hsp70 intron. Other such genetic elements can include, but are not limited to, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

The term recombinant or modified nucleic acids refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

As used herein, the term “upregulation” refers to increased expression (e.g., of mRNA, polypeptides, etc.) relative to expression in a wild type organism (e.g., plant) as a result of genetic modification with a particular emphasis on upregulation in response to a stimulus such as cytokinin signaling. In some embodiments, the increase in expression is a slight increase of about 10% more than expression in wild type. In some embodiments, the increase in expression is an increase of 50% or more (e.g., 60%, 70%, 80%, 100%, etc.) relative to expression in wild type. In some embodiments, an endogenous gene is upregulated. In some embodiments, an exogenous gene is upregulated by virtue of being expressed. Upregulation of a gene in plants can be achieved through any known method in the art, including but not limited to, the use of constitutive promoters with inducible response elements added, inducible promoters, high expression promoters (e.g., PsaD promoter) with inducible response elements added, enhancers, transcriptional and/or translational regulatory sequences, codon optimization, modified transcription factors, and/or mutant or modified genes that control expression of the gene to be upregulated in response to a stimulus such as cytokinin signaling.

Where a recombinant nucleic acid is intended for expression, cloning, or replication of a particular sequence, DNA constructs prepared for introduction into a host cell will typically comprise a replication system (e.g., vector) recognized by the host, including the intended DNA fragment encoding a desired polypeptide, and can also include transcription and translational initiation regulatory sequences operably linked to the polypeptide-encoding segment. Additionally, such constructs can include cellular localization signals (e.g., plasma membrane localization signals). In preferred embodiments, such DNA constructs are introduced into a host cell's genomic DNA, chloroplast DNA or mitochondrial DNA.

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies of the invention disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present invention. One of skill in the art will recognize that any relevant markers available can be utilized in practicing the inventions disclosed herein.

Screening and molecular analysis of recombinant strains of the present invention can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to the subject invention. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. See, e.g., Sambrook et al. (1989) vide infra or Ausubel et al. (1995) Current Protocols in Molecular Biology, John Wiley & Sons, NY, N.Y., for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Because the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

Nucleic acids and proteins of the present invention can also encompass homologues of the specifically disclosed sequences for NIN proteins and NLP proteins. Homology (e.g., sequence identity) can be 50%-100%. In some instances, such homology is greater than 80%, greater than 85%, greater than 90%, or greater than 95%. The degree of homology or identity needed for any intended use of the sequence(s) is readily identified by one of skill in the art. As used herein percent sequence identity of two nucleic acids is determined using an algorithm known in the art, such as that disclosed by Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:402-410. BLAST nucleotide searches are performed with the NBLAST program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (NBLAST and XBLAST) are used. See www.ncbi.nih.gov.

Preferred host cells are plant cells. Recombinant host cells, in the present context, are those which have been genetically modified to contain an isolated nucleic molecule, contain one or more deleted or otherwise non-functional genes normally present and functional in the host cell, or contain one or more genes to produce at least one recombinant protein. The nucleic acid(s) encoding the protein(s) of the present invention can be introduced by any means known to the art which is appropriate for the particular type of cell, including without limitation, transformation, lipofection, electroporation or any other methodology known by those skilled in the art.

Having generally described this invention, the same will be better understood by reference to certain specific examples, which are included herein to further illustrate the invention and are not intended to limit the scope of the invention as defined by the claims.

### EXAMPLES

The present disclosure is described in further detail in the following examples which are not in any way intended to limit the scope of the disclosure as claimed. The attached figures are meant to be considered as integral parts of the specification and description of the disclosure. The following example is offered to illustrate, but not to limit the claimed disclosure.

### Example 1: Upstream Cytokinin Responsive Cis-Elements are Required for NIN Expression in the Pericycle to Allow Full Complementation

The following example describes the identification of cis-regulatory cytokinin responsive elements located in the NIN 5′-upstream region, which are required for nodule primordium formation in M. truncatula. The importance of this region was demonstrated by complementing the M. truncatula nin-1 mutant.

### Materials and Methods

Plant material and growth, hairy root transformation and inoculation with rhizobia: M. truncatula ecotype Jemalong A17 was used as the wild type plant. Agrobacterium rhizogenes (A. rhizogenes) msu440 mediated hairy root transformation was performed as described in Limpens et al., 2004 (Limpens et al., J. Exp. Bot., (2004) 55, 983-992). M. truncatula plants were grown in perlite saturated with low nitrate [0.25 mM Ca(NO₃)₂] containing Färhaeus (Fä) medium at 21° C. and 16 h light/8 h dark regime. Following one week of growth, plants were inoculated with Sinorhizobium meliloti (S. meliloti) RCR2011 constitutively expressing GFP (strain RCR2011.pHC60) or carrying the PronifH:GFP reporter, whereby these reporters were used for experimental purposes but were not required for complementation (OD₆₀₀=0.1, 2 mL per plant). Plants growing on Fä plates were spot-inoculated with 0.5 μL of rhizobium suspension per root.

Constructs: Various segments of the DNA of the NIN gene (including the 3′UTR) and promoter regions were generated with PCR using M. truncatula genomic DNA as a template, Phusion high-fidelity DNA polymerase (Finnzymes) and the primers listed in Table 1. The DNA segments used for pENTR-D-TOPO cloning (Invitrogen) were amplified with a forward primer containing an extra 5′-CACC sequence. Forward primers containing an attB4 site (GGGGACAACTTTGTATAGAAAAGTTGNN, SEQ ID NO:627) and reverse primers with an attB1 site (GGGGACTGCTTTTTTGTACAAACTTGN, SEQ ID NO:628) were used to generate DNA segments for cloning into the vector pDONOR P4-P1 by BP recombination (Invitrogen). The forward primers with attB2 (GGGGACAGCTTTCTTGTACAAAGTGGAA, SEQ ID NO:629) and reverse primers with attB3 (GGGGACAACTTTGTATAATAAAGTTGC, SEQ ID NO:630) were used to amplify DNA fragments for cloning into the vector pDONOR P2-P3. Two rounds of PCR were used to generate three deletions corresponding to the three domains (D1, D2, and D3) in the cytokinin response elements containing (CE) region, as well as deletion of the putative CYCLOPS binding site in the −5 kb region (see, e.g., FIGS. 5A-5C). In the first round of PCR, the two DNA fragments separated by the deletion were amplified with specific primers to introduce 15 bp overhang. The following specific primers were used in the first round of PCR: ProNIN-ΔD1-R and ProNIN-ΔD1-F (to generate a deletion in the D1 domain), ProNIN-ΔD2-R and ProNIN-ΔD2-F (to generate a deletion in the D2 domain), ProNIN-ΔD3-R and ProNIN-ΔD3-F (to generate a deletion in the D3 domain), and ProNIN-ΔCYCLOPS-R and ProNIN-ΔCYCLOPS-F (to generate a deletion in the CYCLOPS binding site) (Table 1). Subsequently, the PCR products were purified and mixed, and 5 μL, of the mixture generated in the first round of PCR was used as a template for the second round of PCR. In the second round of PCR, the primers ProNIN-CE-F and ProNIN-CE-R, ProNIN-5 kb-F and ProNIN-0kb-R, or ProNIN-2.2 kb-F and ProNIN-0kb-R (Table 1) were used to generate a single amplicon with a deletion in the CE, −2.2 kb, or −5 kb region. The Entry vectors were recombined into the modified Gateway binary vector pKGW-RR-MGW (Ovchinnikova et al., Spp. Mol. Plant. Microbe. Interact., (2011) 24, 1333-1344) using Multisite LR recombination (Invitrogen). The constructs generated using the primers listed in Table 1 were ProNIN_{2.2kb}:NIN, ProNIN_{5kb}:NIN, ProNIN_{5kb(Δcyclops)}:NIN, ProNIN_{3C-5kb}:NIN, ProNIN_{CE-5kb}:NIN, ProNIN_{CE-3.5Smin}:NIN, ProNIN_{CE(ΔD1)-5kb}:NIN, ProNIN_{CE(ΔD2)-5kb}:NIN, and ProNIN_{CE(ΔD3)-5kb}:NIN.

TABLE 1
Primer sequences

| Name | Sequence (5′ → 3′) |
|---|---|
| JH5.17-F | GACATCTTTCGTTGGTGGCAA |
| | (SEQ ID NO: 526) |
| JH5.17-R | TCGATGTTTTTCGGGGGTGT |
| | (SEQ ID NO: 527) |
| NINg-F | GGGGACAGCTTTCTTGTACAAAGTGG |
| | AAATGGAATATGGTGGTGGGTTAGTG |
| | (SEQ ID NO: 528) |
| NINg-R | GGGGACAACTTTGTATAATAAAGTTGCGA |
| | ACAAAATAGTTTATGTAATCACAAAGAC |
| | (SEQ ID NO: 529) |
| ProNIN- | CACCGTGGTACCCACTCAATGGTA |
| 2.2kb-F | (SEQ ID NO: 530) |
| ProNIN- | CACCTACTCTATTAGTGCTACCTT |
| 5kb-F | (SEQ ID NO: 531) |
| ProNIN- | CCTTATAATTAAAGTTGTTTCTCAGATC |
| 0kb-R | (SEQ ID NO: 532) |
| ProNIN- | GGGGACAACTTTGTATAGAAAAGTTGGTC |
| 3C-F | ATGGCTTGTCCAACAAC |
| | (SEQ ID NO: 533) |
| ProNIN- | GGGGACTGCTTTTTTGTACAAACTTGCTT |
| 3C-R | TCCCGCATGATACTCAACG |
| | (SEQ ID NO: 534) |
| ProNIN- | GGGGACAACTTTGTATAGAAAAGTTGGCA |
| CE-F | ACAACGCACAACTCGTAC |
| | (SEQ ID NO: 535) |
| ProNIN- | GGGGACTGCTTTTTTGTACAAACTTGGTT |
| CE-R | GCTAACGAGTGCCTTCATG |
| | (SEQ ID NO: 536) |
| ProNIN- | CAATCAGTGTTAACGTTCTATTATACTATA |
| ΔD1-R | (SEQ ID NO: 537) |
| ProNIN- | TATAGTATAATAGAACGTTAACACTGATTG |
| ΔD1-F | (SEQ ID NO: 538) |
| ProNIN- | GTCTCAAGAGCAGTGGATCTGCTTAAGTAA |
| ΔD2-R | (SEQ ID NO: 539) |
| ProNIN- | TTACTTAAGCAGATCCACTGCTCTTGAGAC |
| ΔD2-F | (SEQ ID NO: 540) |
| ProNIN- | TTTTAGTTATAATAGGAACATGTCTGATCA |
| ΔD3-R | (SEQ ID NO: 541) |
| ProNIN- | TGATCAGACATGTTCCTATTATAACTAAAA |
| ΔD3-F | (SEQ ID NO: 542) |
| ProNIN- | GGGCCATCTCTCTGCTTCTACAAATTTTCT |
| ΔCYCLOPS-R | (SEQ ID NO: 543) |
| ProNIN- | AGAAAATTTGTAGAAGCAGAGAGATGGCCC |
| ΔCYCLOPS-F | (SEQ ID NO: 544) |
| NIN- | ATTGCAAGGCGATTTAACCTAACA |
| qPCR-F | (SEQ ID NO: 545) |
| NIN- | GAGAGGGGAAGCTTGAAAAAGAGA |
| qPCR-R | (SEQ ID NO: 546) |
| NF-YA1- | TATGGAGGAGACTCTTGTGG |
| qPCR-F | (SEQ ID NO: 547) |
| NF-YA1- | GGTTGCTTGATGATTTGGTG |
| qPCR-R | (SEQ ID NO: 548) |
| ACTIN2- | TGGCATCACTCAGTACCTTTCAACAG |
| qPCR-F | (SEQ ID NO: 549) |
| ACTIN2- | ACCCAAAGCATCAAATAATAAGTCAACC |
| qPCR-R | (SEQ ID NO: 550) |

Sequences designated in bold were added to primers
for TOPO cloning or BP recombination.

Histological analysis and microscopy: The transgenic roots carrying ProNIN:GUS constructs were incubated in GUS buffer [3% sucrose, 10 mM EDTA, 2 mM k-ferrocyanide, 2 mM k-ferricyanide, 0.5 mg/mL X-Gluc in 0.1M phosphate buffer (pH=7)] at 37° C. for 1-2 hours. Plant tissue embedding in plastic, sectioning and staining were performed as described in Xiao et al. (Xiao et al., Development, (2014) 141, 3517-3528). Sections were analyzed using a DM5500B microscope equipped with a DFC425C camera (Leica). Bright-field and fluorescence images of transgenic roots and nodules were taken using a stereo macroscope (M165 FC, Leica). Confocal images were taken using a SP8 (Leica) microscope. 488 nm and 543 nm excitation wavelengths were used for GFP and propidium iodide respectively.

RNA isolation and qRT-PCR: RNA was isolated from one week old M. truncatula A17 and daphne-like roots using the EZNA Plant RNA mini kit (Omega Bio-tek, Norcross, Ga., USA). 1 μg of isolated RNA was used for cDNA synthesis with the iScript cDNA synthesis kit (Bio-Rad). Real-time qPCR was performed in 10 μl reactions using SYBR Green Supermix (Bio-Rad) and a CFX real-time system (Bio-Rad). Gene expression levels were determined using the primers listed in Table 1 designated with “qPCR” in the primer name. The gene expression levels were normalized using ACTIN2 as a reference gene.

Expression induction using cytokinin: To determine whether gene expression was induced by cytokinin, roots of M. truncatula A17 (WT) and daphne-like were treated with either 10⁻⁷M benzylaminopurine (BAP) or water for 16 hours. Then, qRT-PCR analysis of the genes NIN and NF-YA1 was performed using the primers listed in Table 1. This experiment was repeated for a total of three biological replicates.

Quantification of colonies, infection threads, and nodules: To quantify the number of curled root hairs containing colonies or infection threads, more than 20 transgenic roots (5-10 cm long) were cut into fragments of ˜1 cm and randomly selected for counting. To quantify the nodule number per root, 5-10 cm long transgenic roots were selected.

RNA in situ hybridization: M. truncatula roots were fixed with 4% paraformaldehyde mixed with 3% glutaraldehyde in 50 mM phosphate buffer (pH=7.4) and embedded in paraffin (Paraplast X-tra, McCormick Scientific). Root sections of 7 μm were prepared by RJ2035 microtome (Leica). RNA in situ hybridization was conducted using Invitrogen ViewRNA™ ISH Tissue 1-Plex Assay kits (ThermoFisher Scientific) according to directions in the user manual (Available at cdn.panomics.com under the directory downloads/17400RevF %20140822_ViewRNA%20ISH%20Tissue%201-Plex.pdf). RNA ISH probe sets were designed and synthesized by request at ThermoFisher Scientific. Catalog numbers of probes were VF1-20312 for Mt NIN, VF1-6000865 for Mt CRE1, VF1-6000866 for Mt RR1, and VF-20311 for Mt NF-YA1. A typical probe set consisted of ˜20 pairs of oligonucleotide probes (20 nt long) that hybridized to specific regions across the target mRNA. Each probe was composed of a region of ˜20 nucleotides, a short linker region and a tail sequence. The two tail sequences (double Z) together formed a site for signal amplification. Such design ensured increased background control by reducing the chance of a nonspecific hybridization event being amplified. For the nodulation specific genes, non-inoculated roots were used as a negative control. For ISH with CRE1 and RR1 performed on non-inoculated roots, an ENOD2 (nodule specific gene) probe set was used as a negative control. Images were taken with an AU5500B microscope equipped with a DFC425c camera (Leica).

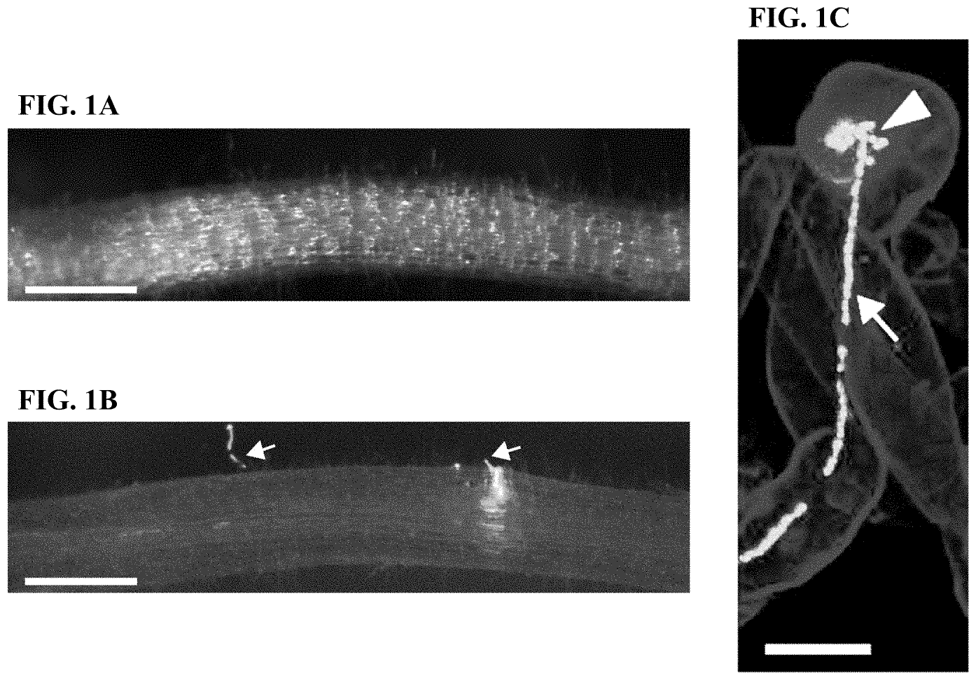

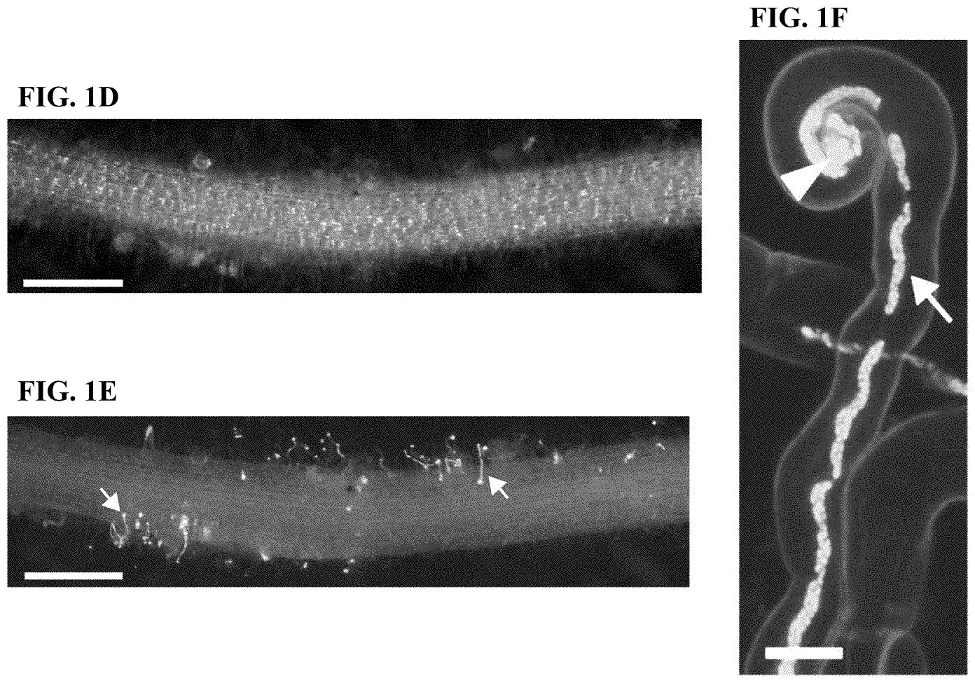

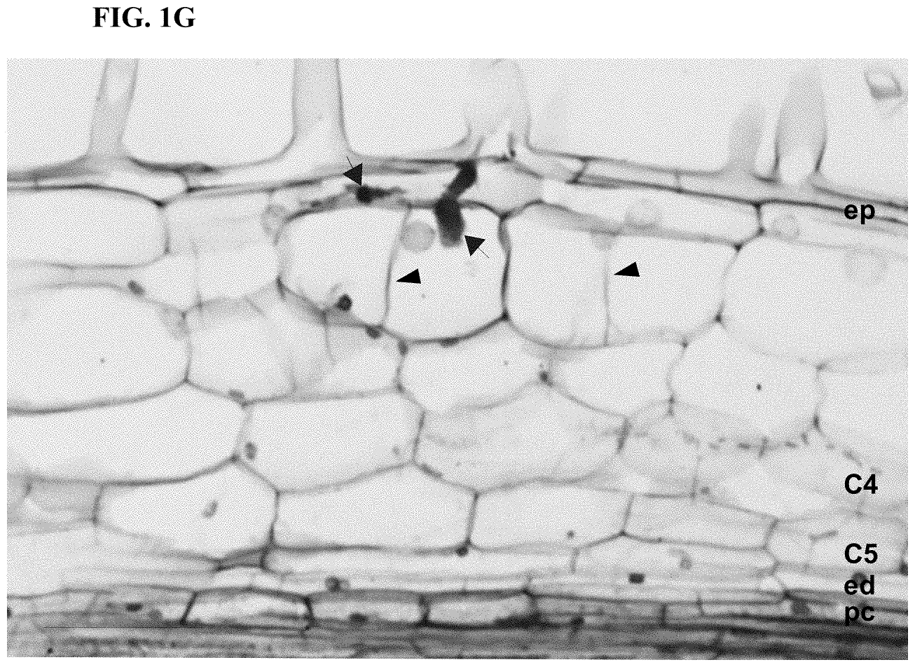

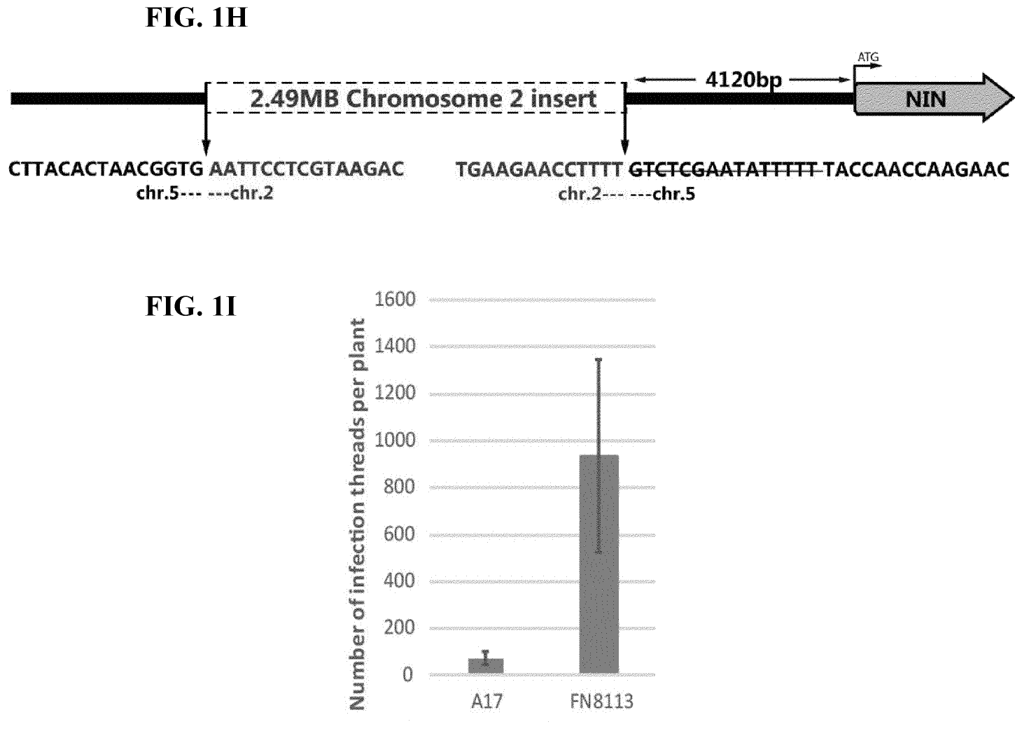

Map-based cloning of daphne-like: A segregating F2 population was made from a cross between M. truncatula FN8113 (cv Jemalong A17) and M. truncatula Jemalong A20 (118 plants). This population showed an approximate 3:1 ratio of Nod+:Nod− plants (118 F2 plants; 84 Nod+: 34 Nod−). The 3:1 ratio indicated that FN8113 had a single recessive mutation responsible for its Nod− phenotype. DNA was extracted using the standard CTAB DNA miniprep method. Simple sequence repeat markers (SSR) based on Mun et al. (Mun et al., Genetics, (2006) 172, 2541-2555) were first used to determine the global chromosomal location of the FN8113 locus. The mutation was shown to be located at the end of chromosome 5, where NIN is located. Subsequently, additional SSR markers were developed for the FN8113 locus on chromosome 5, and used for chromosome walking. PCR was performed using 100 ng of genomic DNA, and analyzed on 2.5% agarose gels. The SSR marker JH5.17 (Table 1) on BAC clone CU424494 showed the closest linkage to the FN8113 locus. No cross-overs were found at the distal end of chromosome 5. Next, whole genome sequencing (Illumina Hiseq2000, paired-end) was used to identify mutations in the genomic region identified from the genetic mapping. This revealed a translocation of a ˜2.49 Mbp region from chromosome 2 into chromosome 5, inserted 4120 bp upstream of the NIN start codon (−4120). In addition, a small deletion of 15 bp between −4121 and −4135 was detected (FIG. 1H). No mutations were found in the NIN coding sequence. Genomic sequence of the mutated region is provided as SEQ ID NO:525. Cleaned DNA sequence reads were mapped against the M. truncatula genome published by Young et al. (Young et al., Nature, (2011) 480, 520-524) using the bwa mem algorithm (Li and Durbin, Bioinformatics, (2010) 26, 589-595). Clipped reads and mismapped mate pairs revealed an inter-chromosomal translocation, which was further confirmed by aligning reads spanning the mutation to the genome using BLASTN.

Alignment of the upstream regions of NIN: Most of this work was carried out using Geneious v8.1.9 (https://www.geneious.com) (Kearse et al., Bioinformatics, (2012) 28, 1647-1649). The M. truncatula NIN protein sequence was BLASTed against custom BLAST databases using Geneious v8.1.9 (Altschul et al., J. Mol. Biol., (1990) 215, 403-410; Kearse et al., Bioinformatics, (2012) 28, 1647-1649). A diverse selection of legume species with a good quality of publicly available genomic sequences were used: Medicago truncatula (Young et al., Nature, (2011) 480, 520-524), Lotus japonicus (Sato et al., DNA Res., (2008) 15, 227-239), Arachis duranensis (Bertioli et al., Nat. Genet., (2016) 48, 438-446), Cicer arietinum (Varshney et al., Nat. Biotechnol., (2013) 31, 240-246), Glycine max (Schmutz et al., Nature, (2010) 463, 178-183), Lupinus angustifolius (Hane et al., Plant Biotechnol. J., (2017) 15, 318-330), Cajanus cajan (Varshney et al., Nat. Biotechnol., (2012) 30, 83-89), and Trifolium pratense (De Vega et al., Sci. Rep., (2015) 5). Selected NIN scaffolds (Table 2) and up to 80 kb upstream and 10 kb downstream of NIN were extracted from the genomes of these legume species. Selected sequences were custom aligned using mVISTAs web based alignment tool (http://genome.lbl.gov/vista/mvista; Frazer et al., Nucleic Acids Res., (2004) 32, 273-279). The alignment program selected was the shuffle-lagan global alignment program which detects rearrangements (Brudno et al., Bioinformatics, (2003) 19, i54-i62). Apart from this larger scale alignment, individual alignments were made using MAUVE as a Geneious plugin (Darling et al., Genome Res., (2004) 14, 1394-1403), which allowed for more precise determination of conserved sequences relative to the NIN start codon in all species. A complete overview of detected conserved regions can be found in Table 2.

TABLE 2
Sequence information of aligned species

| | | | | Putative | | | |
|---|---|---|---|---|---|---|---|
| | | Pseudo- | | CYCLOPS | Location of three conserved regions (3C) (−bp) † | | |
| | Genome | chromosome/ | NIN gene | binding site | | Region 2/ | |
| Species | version | scaffold id | annotation | (−bp) † | Region 1 | CE Region | Region 3 |
| Arachis | Aradu_v1.0 | Aradu.A07 | Aradu.46M2Y | 1102 . . . 1114 | 12755 . . . 12939 | 13822 . . . 14280 | ND‡ |
| duranensis | | | | | | | |
| Cicer | Cicer_ | Ca2 | Ca_09832 | 1467 . . . 1481 | 12144 . . . 12314 | 12503 . . . 13011 | 14593 . . . 15207 |
| arietinum | arietinum_ | | | | | | |
| | GA_v1.0 | | | | | | |
| Lotus | Lj3.0 | Chr2 | Lj2g3v3373100 | 945 . . . 957 | 42126 . . . 42392 | 44723 . . . 45273 | 48459 . . . 48904 |
| japonicus | | | | | | | |
| Glycine max* | Wm82.a2.v1 | Gm02 | Glyma.02g311000. | 640 . . . 652 | 9258 . . . 9544 | 11271 . . . 11783 | ND‡ |
| | Wm82.a2.v1 | Gm04 | Glyma.04g000600 | 2241 . . . 2257 | 8536 . . . 8814 | 10738 . . . 11278 | ND‡ |
| Lupinus | Lupinus_ | NLL-02 | Lup019181.1 | 886 . . . 900 | 4800 . . . 5162 | 6439 . . . 6774 | ND‡ |
| angustifolius* | angustifolius_ | | | | | | |
| | pschrom_v1.0 | | | | | | |
| | | NLL-011 | Lup029716.1 | 590 . . . 614 | 4111 . . . 4507 | 4617 . . . 4874 | ND‡ |
| Cajanus | PigeonPea. | Scaffold133201 | C.cajan_37712 | 1396 . . . 1410 | 18677 . . . 18772 | 23796 . . . 24273 | ND‡ |
| cajan* | scafSeq. | | | | | | |
| | LG_V5.0 | | | | | | |
| | | Scaffold132542 | C.cajan_33924 | 7032 . . . 7046 | 17957 . . . 19089 | 23574 . . . 24225 | 26.361 . . . 26599 |
| Trifolium | redclover_v2.1 | Tp57577_ | TP57577_TGAC_ | 2216 . . . 2230 | 12254 . . . 12463 | 15497 . . . 16132 | 17392 . . . 18136 |
| pratense | | TGAC_v2_LG4 | v2_gene_18624 | | | | |
| Medicago | JCVI.Medtr. | Chr5 | Medtr5g099060 | 3026 . . . 3040 | 15769 . . . 15985 | 17611 . . . 18083 | 18654 . . . 19153 |
| truncatula | v4.20130313 | | | | | | |

*Glycine max, Lupinus angustifolius and Cajanus cajan have two NIN genes. Glycine max Gm04, Lupinus angustifolius NLL-011, and Cajanus cajan Scaffold132542 were not used for alignment (below).
† The relative position of conserved regions compared to M. truncatula was scored for each scaffold based on MAUVE alignments. Regions are annotated relative to NIN start codon.
‡ND, not detected.

Alignment of CE regions and prediction of binding sites: Detected conserved sequences of CE regions for selected scaffolds (Table 2) were aligned using MAFFTv7.017 as Geneious plugin (Katoh, Nucleic Acids Res., (2002) 30, 3059-3066). Conserved binding sites were predicted using PlantPAN2.0 (Chow et al., Nucleic Acids Res., (2016) 44, D1154-D1164). Some sites were manually added based on homology with previously published putative B-type RR binding sequences (Heyl and Schmülling, Curr. Opin. Plant Biol., (2003) 6, 480-488; Hosoda et al., Plant Cell, (2002) 14, 2015-2029; Imamura et al., Plant Cell Physiol., (2003) 44, 122-131).

### Results

Isolation of a novel M. truncatula nin mutant in which infection and nodule organogenesis are uncoupled: Nod-mutant FN8113 was identified by screening a plant population obtained from fast neutron bombardment mutagenized M. truncatula seeds (Noble Research Institute, LLC., Ardmore USA). This mutant was named daphne-like because its phenotype was strikingly similar to that of the L. japonicus daphne mutant. As shown in FIGS. 1D and 1E, three weeks after inoculation with S. meliloti, daphne-like showed excessive infection thread formation relative to WT (FIGS. 1A and 1B), but nodulation was strongly impaired relative to wild type. The root hair curling of daphne-like (FIG. 1F) resembled that of WT (FIG. 1C), in that entrapped bacteria formed colonies and infection threads were formed. The infection thread numbers in WT roots and daphne-like roots were quantified two weeks after inoculation; this showed that infection thread number was more than ten-fold higher in daphne-like than in WT (FIG. 1I). The majority of infection threads were arrested in daphne-like root hairs, but longitudinal sections of roots showed that a few infection threads (indicated by arrows) could reach cortical cell layers (FIG. 1G). FIG. 1G also shows that occasionally some cortical cells divided locally around infection threads (indicated by arrow heads). However, cell divisions were not induced in the inner root cell layers where nodule primordia are initiated in WT plants.

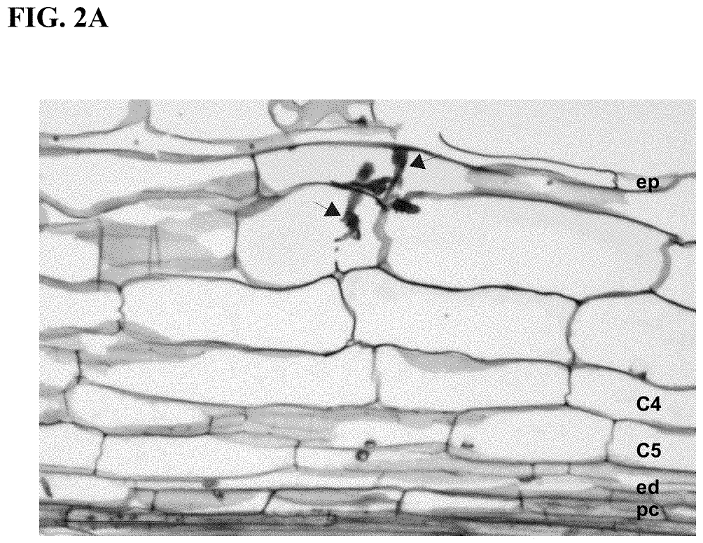

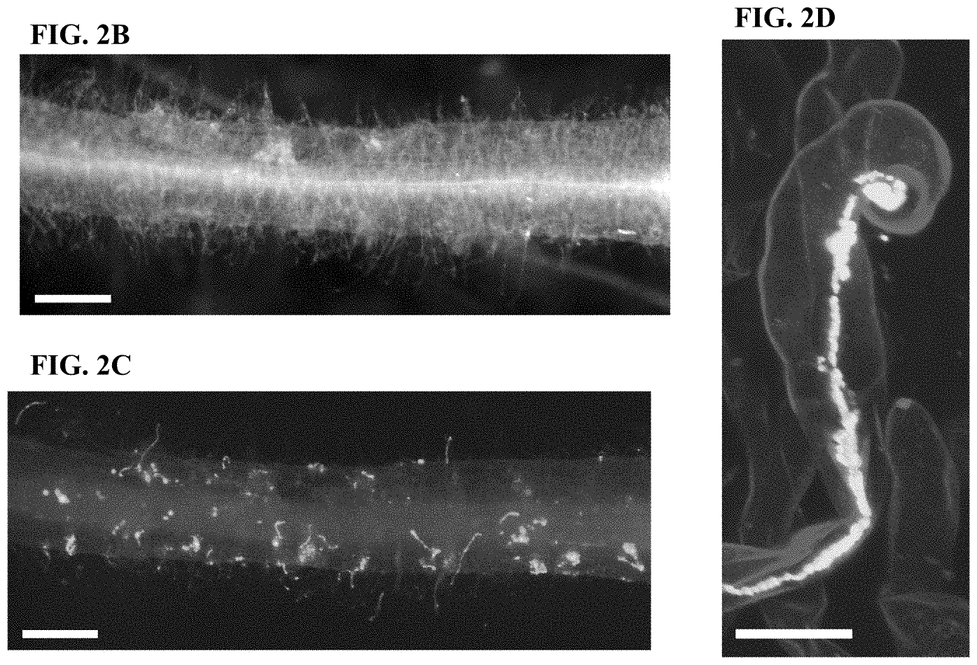

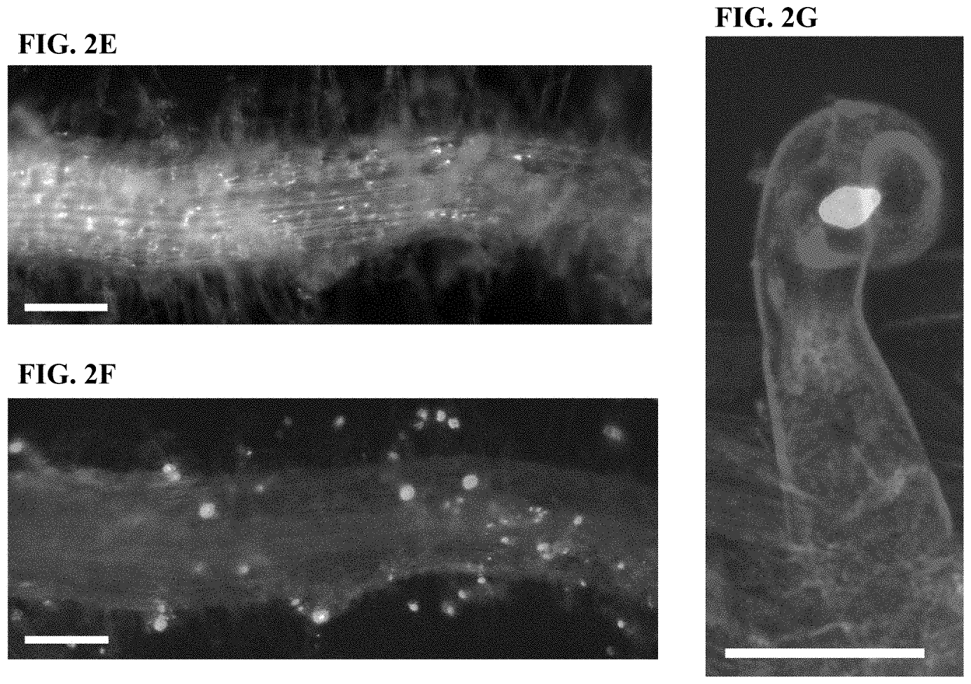

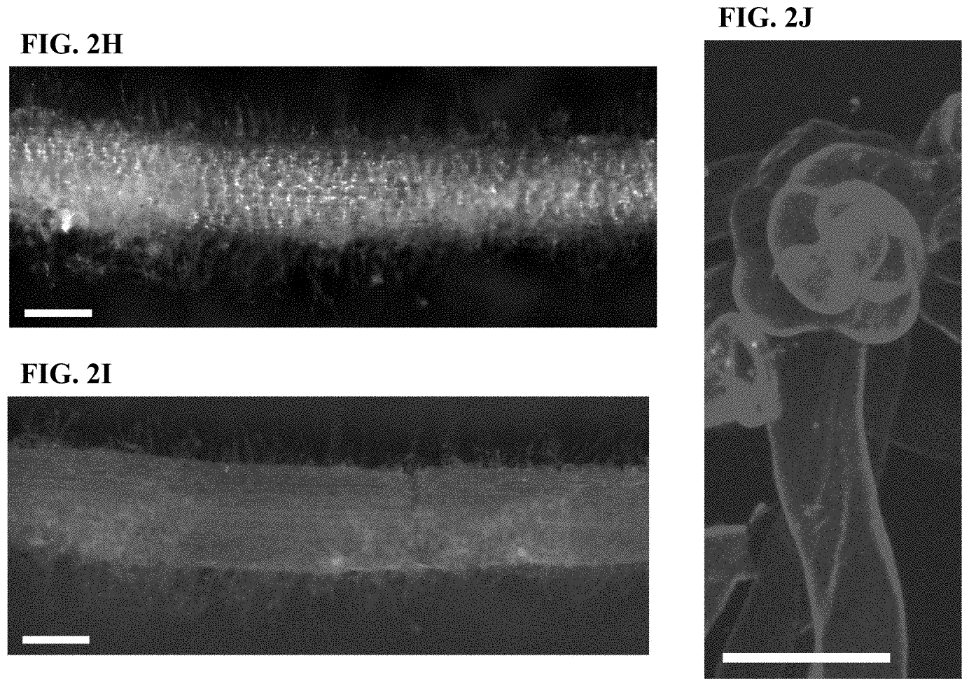

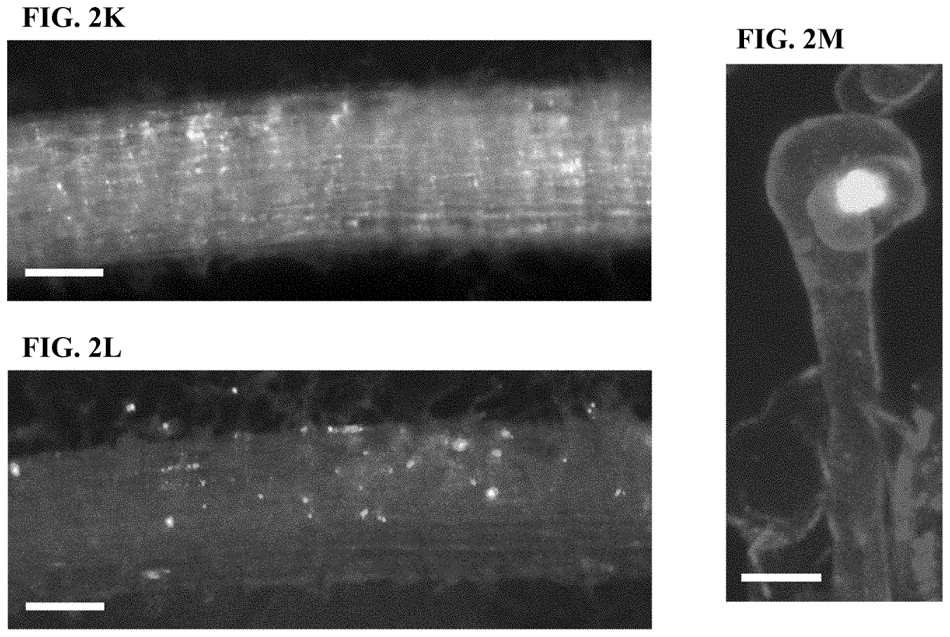

The 5 kb upstream region of Mt NIN contains discrete regulatory sequences for root hair curling and infection: The phenotype of daphne-like indicated that NIN regulatory sequences required for primordium formation were located more than 4120 bp upstream of its start codon. In addition, the phenotype indicated that the regulatory sequences located within this 4120 bp region were sufficient for proper root hair curling and infection thread formation. To confirm this, the 5 kb region upstream of the start codon was used to drive expression of NIN. ProNIN_{5kb}:NIN was introduced into M. truncatula nin-1 (null mutant) (Marsh et al., Plant Physiol., (2007) 144, 324-335) roots by A. rhizogenes-mediated root transformation. Four weeks post-inoculation (4 wpi), 41 out of 44 analyzed transgenic roots showed excessive infection thread formation (FIGS. 2B-2D). Despite the numerous infections, these roots did not form nodules, except one root on which 4 nodules were observed (FIGS. 2B-2D). Longitudinal sections of infected transgenic roots confirmed that cell divisions were not induced in pericycle, endodermis, and inner cortical cell layers (FIG. 2A). FIG. 2A also shows that infection threads were arrested in the epidermis, but occasionally reached the cortex. These data demonstrate that the 5 kb promoter region is sufficient for infection thread formation, but it lacks regulatory sequences for primordium formation.

It was previously known that a single putative CYCLOPS/Mt IPD3 binding site was located about −3 kb upstream of the start codon (FIGS. 4A and 5D, Table 2) (Singh et al., Cell Host Microbe, (2014) 15, 139-152). Therefore, a construct was developed where the −2.2 kb upstream region was used to drive NIN expression (the ProNIN_{2.2kb}:NIN construct) to determine whether the function of NIN in the epidermis fully depended on the putative CYCLOPS binding site (FIGS. 5A-5C). This construct was then compared to the ProNIN_{5kb}:NIN construct and empty vector by transforming nin-1 using A. rhizogenes mediated hairy root transformation. As shown in FIGS. 21I-2J, the nin-1 null mutant transformed with empty vector had excessive root hair curling but failed to form infection threads and micro-colonies. All 37 analyzed roots transformed with the ProNIN_{2.2kb}:NIN construct showed tight root hair curls entrapping bacterial colonies, but infection threads were rare (FIGS. 2E-2G). In ProNIN_{2.2kb}:NIN transgenic roots, 298 curled root hairs containing a bacterial colony were analyzed, but only ˜3% had an infection thread. These data show that root hair curling and establishment of infection chambers do not rely on the putative CYCLOPS binding site. In contrast, ˜70% of curled root hairs formed infection threads in ProNIN_{5kb}:NIN transgenic roots (n=324). These results indicated that the −5 kb to −2.2 kb region contained regulatory sequences important for infection thread formation. The observed phenotype was reminiscent of that of L. japonicus and M. truncatula cyclops-3lipd3-2 mutants (Yano et al., Proc. Natl. Acad. Sci. U.S.A., (2008) 105, 20540-20545; Horvath et al., Mol. Plant-Microbe Interact., (2011) 24, 1345-1358). The L. japonicus and M. truncatula cyclops-3lipd3-2 mutants have a phenotype where bacterial colonies are formed in tightly curled root hairs, but infection threads are not formed. Taken together, these results indicate that the −2.2 kb upstream region is sufficient for activating NIN expression in the epidermis at expression levels that result in tight root hair curling. This tight curling allows rhizobia to form a colony inside the pocket of the curl. However, additional regulatory sequences located between −5 kb and −2.2 kb upstream, probably involving the putative CYCLOPS binding site, are required for efficient infection thread formation. To test this, nin-1 roots transformed with NIN driven by the −5 kb promoter in which the putative CYCLOPS binding site was deleted (ProNIN5kb(Δcyclops):NIN) were tested (FIGS. 2K-2M). By deleting the putative CYCLOPS binding site, the number of curled root hairs with a microcolony that formed an infection thread dropped from 70% to 7% (n=434). This result showed that the putative CYCLOPS binding site within the NIN promoter was essential for efficient infection thread formation.

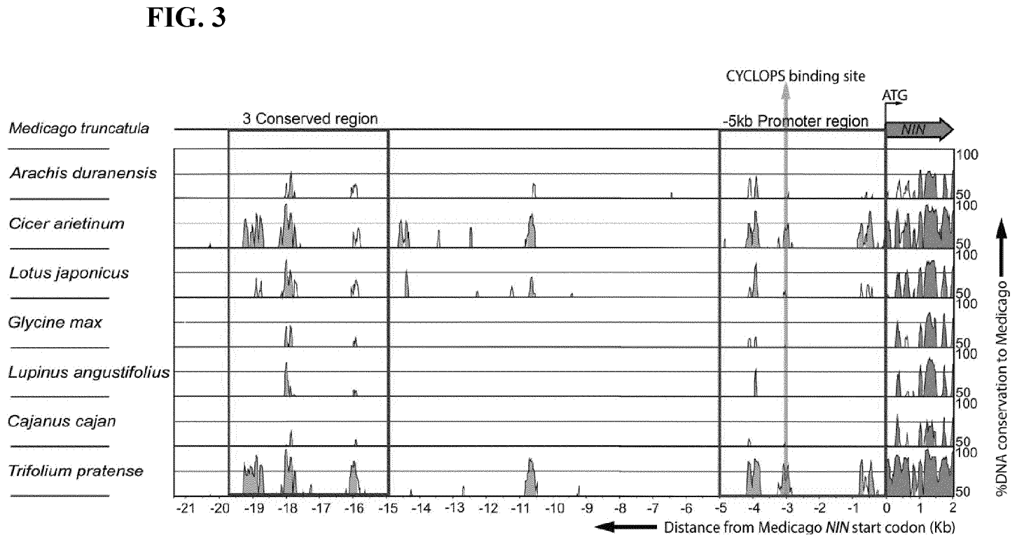

A conserved region with putative cytokinin response elements is located ˜18 kb upstream of the Mt NIN coding region: As described above, daphne-like as well as nin-1 transformed with ProNIN_{5kb}:NIN were able to induce infection thread formation but not nodule primordium formation. In order to identify remote regulatory regions located upstream of the −5 kb region, the genomic DNA sequences spanning from the start of the NIN coding region to the first upstream gene, of 8 legume species (M. truncatula, L. japonicus, Arachis duranensis, Cicer arietinum, Glycine max, Lupinus angustifolius, Cajanus cajan and Trifolium pratense) were compared. DNA sequences with 3 conserved regions (3C) far upstream of the NIN start codon were identified (FIGS. 3 and 4A, Table 2). In M. truncatula, 3C was located 15-20 kb upstream of the NIN start codon, and in L. japonicus it was located between 42-49 kb upstream (Table 2). The level of similarity observed in conserved parts of 3C were similar to those observed in the NIN coding region (FIG. 3). The second region in 3C was the most conserved and included about 10 putative B-type cytokinin signaling RESPONSE REGULATOR (RR) binding sites (FIGS. 3, 4A, and 5A-5C) (Sheen, Science, (2002) 296, 1650-1652; Heyl and Schmülling, Curr. Opin. Plant Biol., (2003) 6, 480-488; Hosoda et al., Plant Cell, (2002) 14, 2015-2029; Imamura et al., Plant Cell Physiol., (2003) 44, 122-131). This region was therefore named the cytokinin response elements containing (CE) region.

The CE region contains regulatory elements required for nodule organogenesis: To determine whether the 3C region (˜4 kb) contained regulatory sequences for nodule primordium formation, 3C was fused to the (upstream) −5 kb region (ProNIN_{3C-5kb}:NIN), as the latter was found to be sufficient for infection. ProNIN_{3C-5kb}:NIN was introduced into nin-1 by A. rhizogenes mediated hairy root transformation. As shown in FIG. 4B, 21 out of 26 analyzed transgenic roots formed ˜8 nodules per root. As described above, the CE region (˜1 kb) was found to contain several putative cytokinin response elements. In order to determine whether the CE region was sufficient to trigger primordium formation, nin-1 was transformed with the CE region fused to the −5 kb region driving NIN (ProNIN_{CE-5kb}:NIN). As shown in FIG. 4B, 18 out of 37 transgenic roots formed ˜8 nodules per root. This data demonstrates that the CE region contains regulatory sequences required for primordium formation. Further, the number of nodules formed on ProNIN_{CE-5kb}:NIN expressing roots was similar to that of wild type roots transformed with an empty vector (control) (FIG. 4B). Thus, the data further demonstrates that the autoregulation of nodulation (AON) mechanism is also activated (Soyano et al., Proc. Natl. Acad. Sci. U.S.A., (2014) 111, 14607-14612).

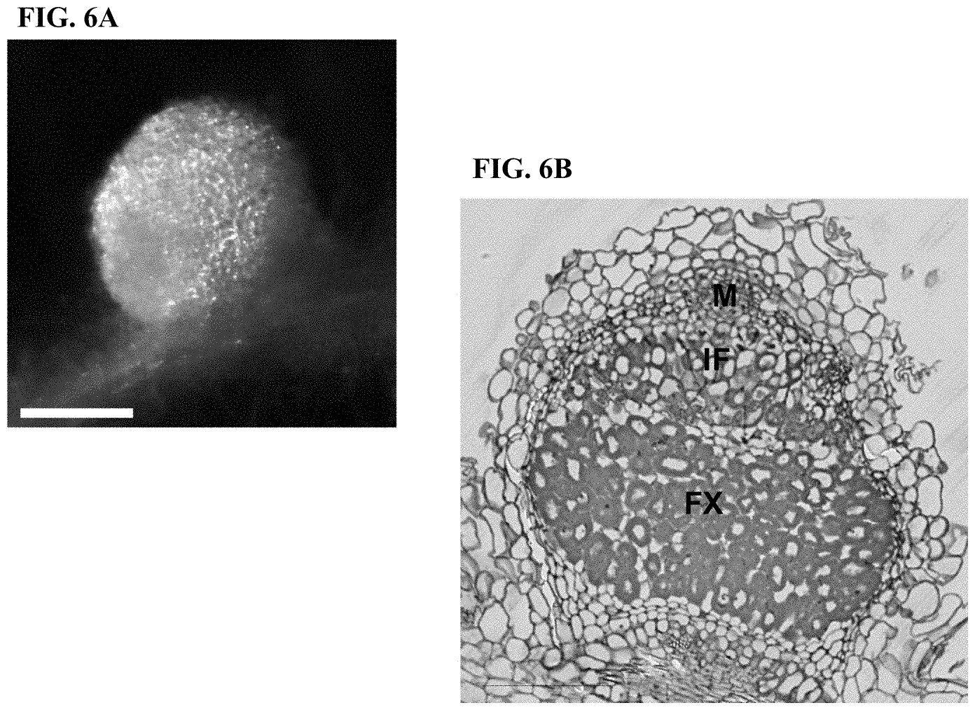

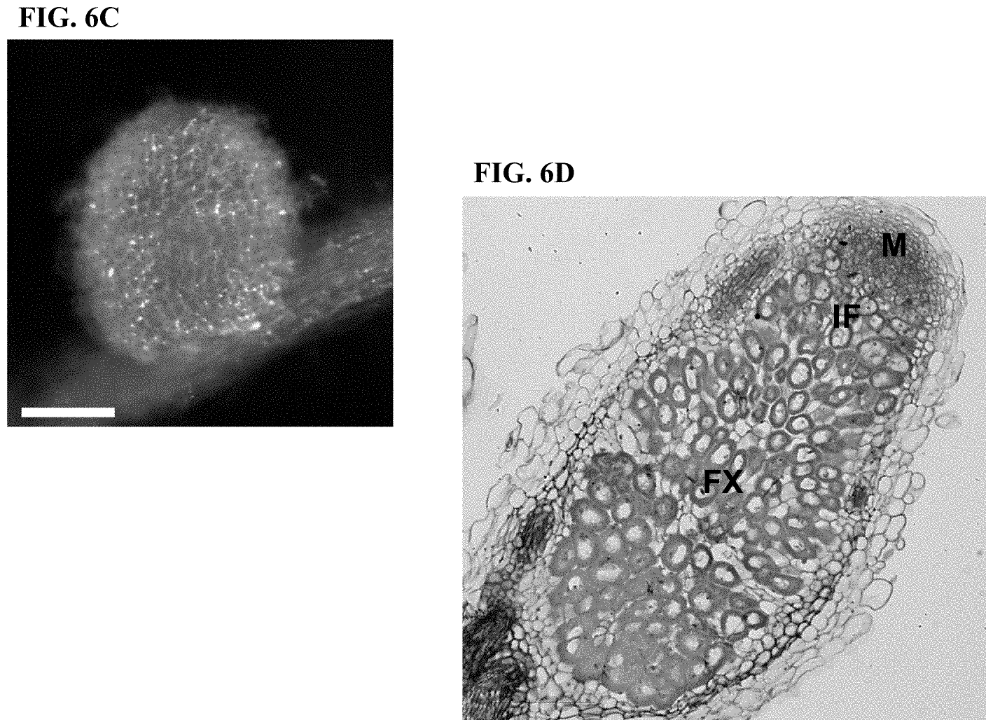

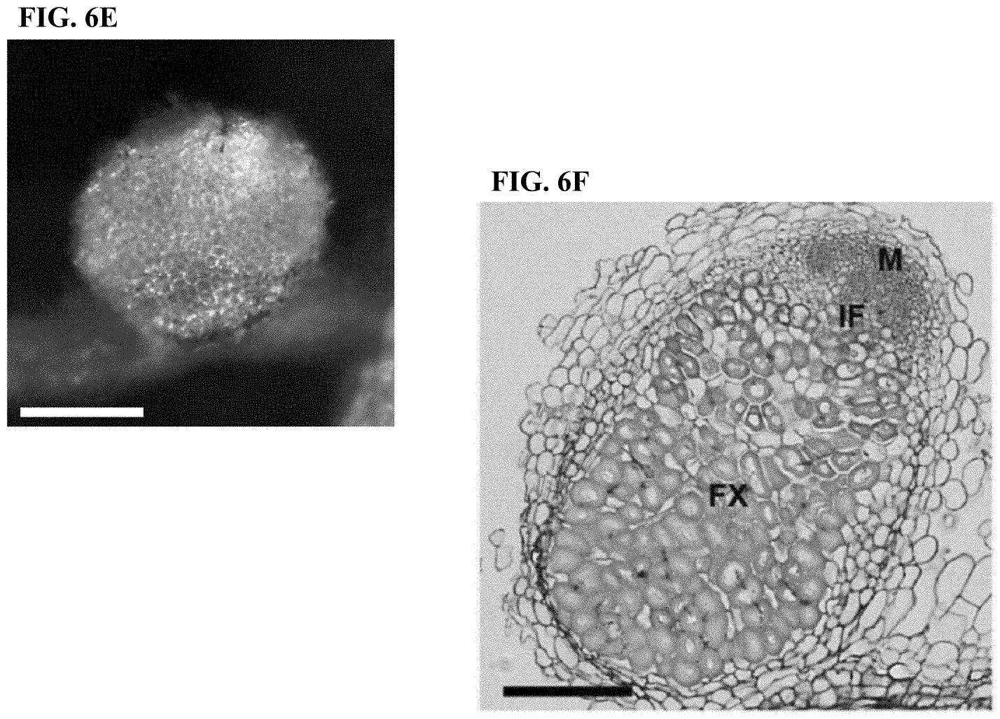

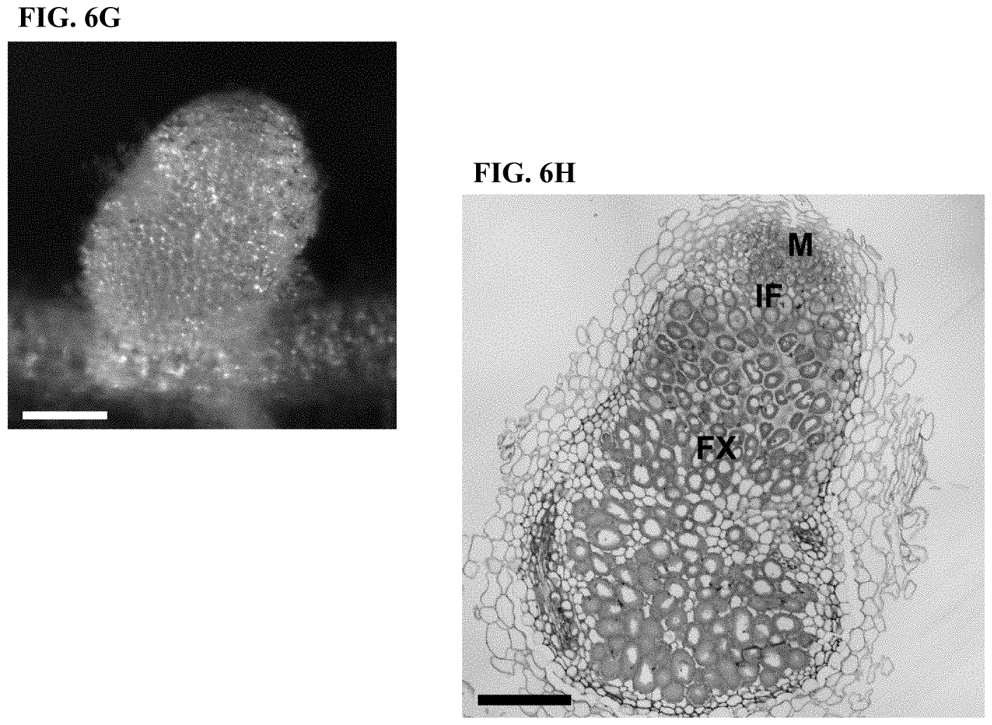

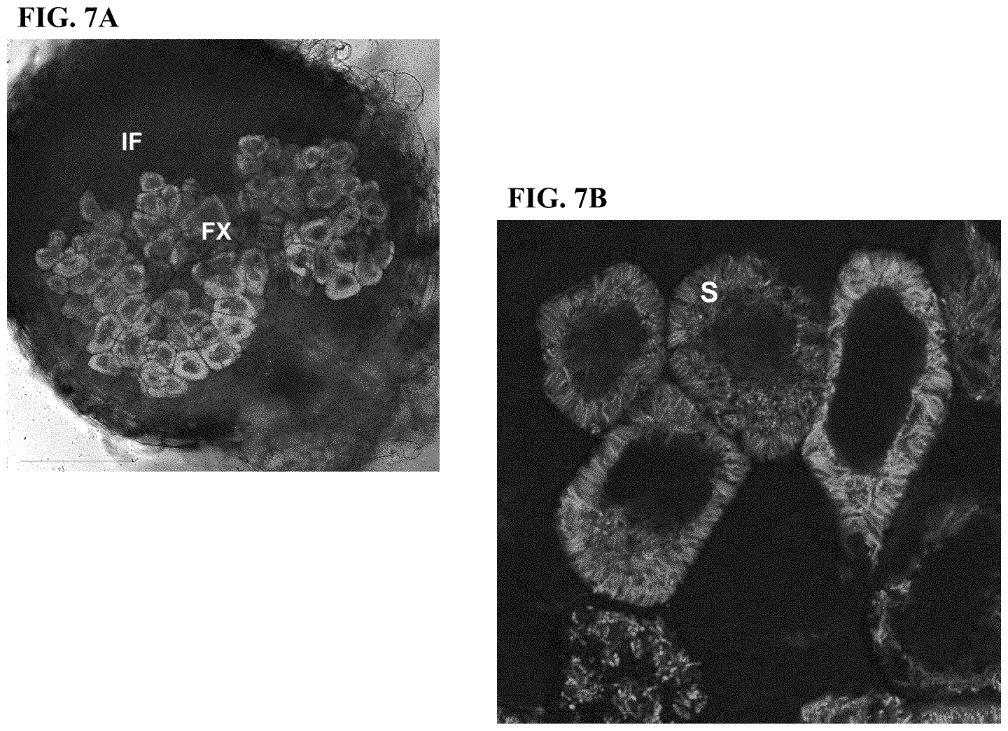

Pink nodules were formed on nin-1 roots transformed with either ProNIN_{3C-5kb}:NIN or ProNIN_{CE-5kb}:NIN. FIGS. 6A and 6C demonstrate that pink nodules were formed on transgenic roots of nin-1. The pink coloration is from leghemoglobin, and is a hallmark of nodules that are actively fixing nitrogen. FIGS. 6B and 6D show longitudinal sections of those same nodules, which displayed normal nodule zonation, including meristem (M), infection zone (IF), and fixation zone (FX). Nodules induced on ProNIN_{CE-5kb}:NIN transgenic nin-1 roots by inoculation with S. meliloti carrying the PronifH:GFP reporter showed that nifH was expressed in the fixation zone (FIGS. 7A and 7B). Thus, CE in combination with the −5 kb region was sufficient to induce wild type-like nodule organogenesis.

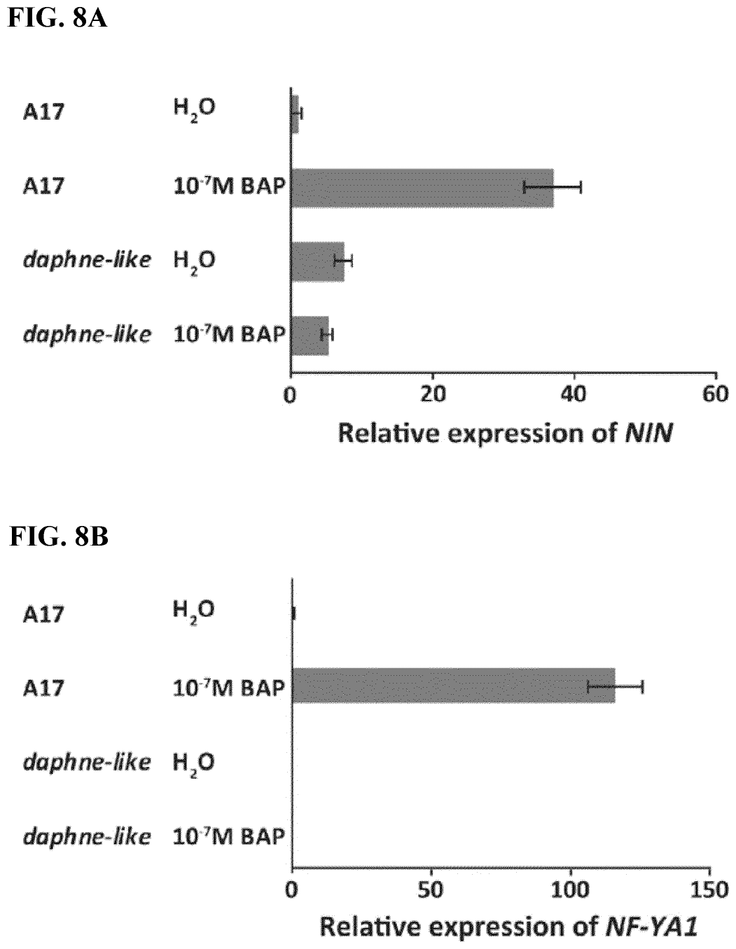

The daphne-like CE region containing a 2.49 Mbp insertion was unable to contribute to the correct expression of NIN, further indicating the importance of the cytokinin-responsive CE region: Pink nodules were formed on daphne-like roots transformed with ProNIN_{CE-5kb}:NIN (FIG. 6E). 15 of 17 analyzed transgenic roots at 4 wpi formed on average of about seven nodules per root, and the excessive infection phenotype in the daphne-like background was rescued in 11 of these 17 transgenic roots. FIG. 6F shows a longitudinal section of those same nodules, which displayed normal nodule zonation, including meristem (M), infection zone (IF), and fixation zone (FX). This result showed that the daphne-like phenotype was likely caused by the 2.49 Mbp insertion that interfered with the function of the CE region. Thus, it was tested whether the CE region was sufficient to complement nodule organogenesis in daphne-like. To do this, a minimal CaMV 35S promoter (−46 bp) (Benfey and Chua, Science, (1990), 250:959-966) fused to the CE region (ProNINCE-35Smin:NIN) was used to transform daphne-like roots. FIG. 6G demonstrates that pink nodules were formed on the transgenic roots, while FIG. 6H shows longitudinal sections of these same nodules, which displayed normal nodule zonation. 37 out of 45 transgenic daphne-like roots formed on average four nodules per root at 4 wpi. This indicated that the CE region was sufficient to induce nodule organogenesis, but in combination with the −5 kb region more nodules (about seven per root) could be formed. Because the ability to form nodules could be rescued in daphne-like by the CE region driving NIN expression, it seemed likely that the CE region in daphne-like could not control the expression of NIN and further could not be induced by cytokinin. Therefore, the induction of NIN expression by cytokinin and water (as control) in wild type (Jemalong A17) and daphne-like was compared. Compared with the control, 16 hours after 10-7M benzylaminopurine (BAP) application, NIN expression level increased 37 folds and NF-YA1 expression level increased 116 folds in wild-type, while both NIN and NF-YA1 expression levels in daphne-like were not changed (FIGS. 8A-8B). This showed that the CE region was required for the induction of NIN expression by cytokinin, and that the CE region in daphne-like was unable to contribute to the correct expression of NIN.

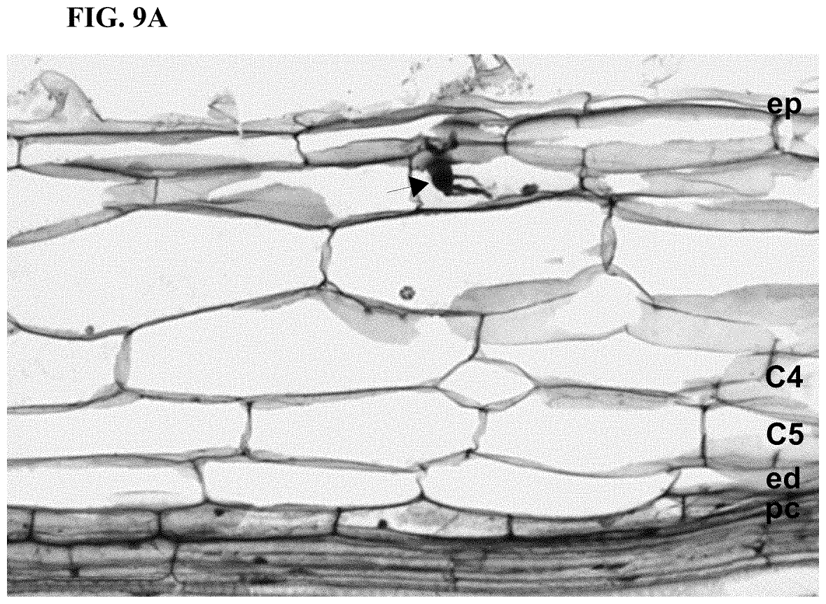

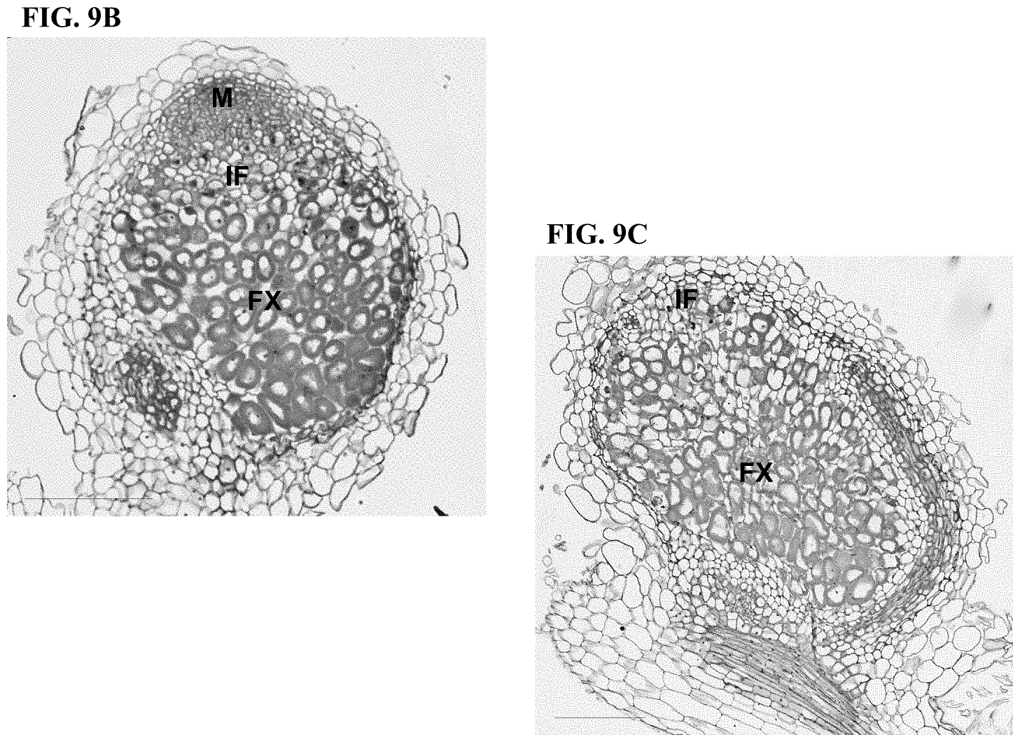

A domain with 6 putative cytokinin response elements is essential for nodule primordium formation: Cytokinin was known to be a positive regulator of nodule primordium formation (Suzaki et al., Front. Plant Sci., (2013) 4, 1-6). Therefore, experiments to determine whether the putative cytokinin response elements within the CE region were essential for primordium formation were conducted. To this end, several deletions in the CE region were made. The CE region contains a 472 bp region that is highly conserved in all 8 studied legume species (FIGS. 3, 4A, and 5A-5C). The 472 bp region was divided into 3 parts: domain 1 to 3 (D1 to D3). D1 and D3 were found to contain six and three putative cytokinin response elements respectively, whereas D2 contained a putative AP2 binding site as well as a single cytokinin response element (FIGS. 4A and 5A-5C). Transcription factors of the AP2 family, including ERN (Ethylene response factor Required for Nodulation) are involved in regulating nodulation (Andriankaja et al., Plant Cell, (2007) 19, 2866-2885; Middleton et al., Plant Cell Online, (2007) 19, 1221-1234; Wang et al., Plant Cell, (2014) 26, 4782-4801). To investigate their respective contribution to nodule primordium formation, D1, D2 or D3 were separately deleted from the 1 kb CE region (FIGS. 4A and 5A-5C), and these modified CE regions were fused to the −5 kb region to drive NIN expression. The 3 constructs were introduced into nin-1 by A. rhizogenes mediated root transformation. As shown in FIGS. 4B and 9A, deletion of D1 eliminated the ability to form nodules, whereas deletion of D2 had no significant effect on nodulation (FIGS. 4B and 9B). Deletion of D3 caused a reduction of the relative number of roots with nodules from 49% to 21% and also reduced the average nodule number per root from 8 to 5.4 (FIGS. 4B and 9C). These results showed that at least regulatory sequences in D1 were essential for NIN-controlled nodule primordium formation. Further, the data indicated that the putative cytokinin response elements within D1 were responsible for NIN controlled nodule primordium formation. In contrast, the putative AP2 binding site in D2 was not essential for nodule organogenesis.

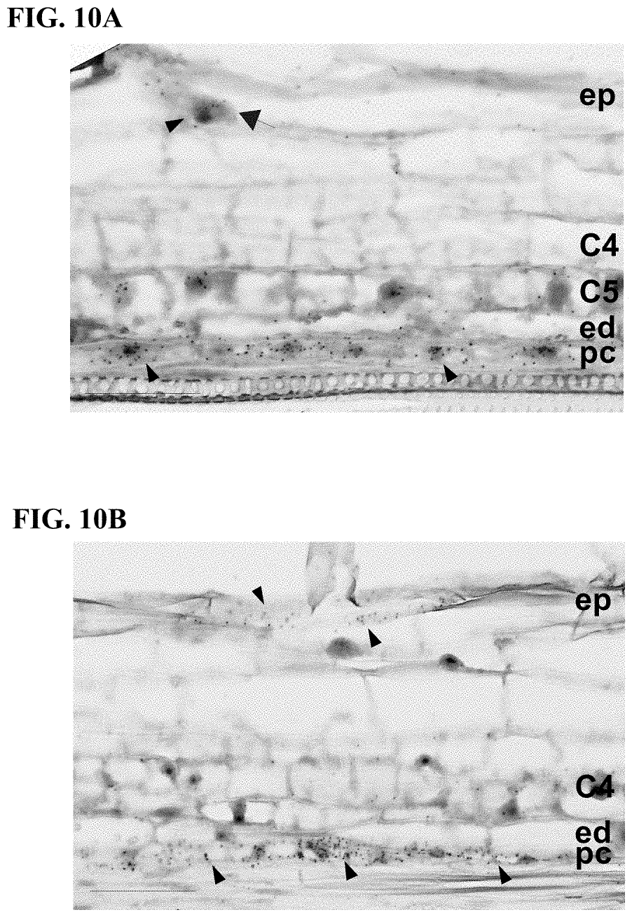

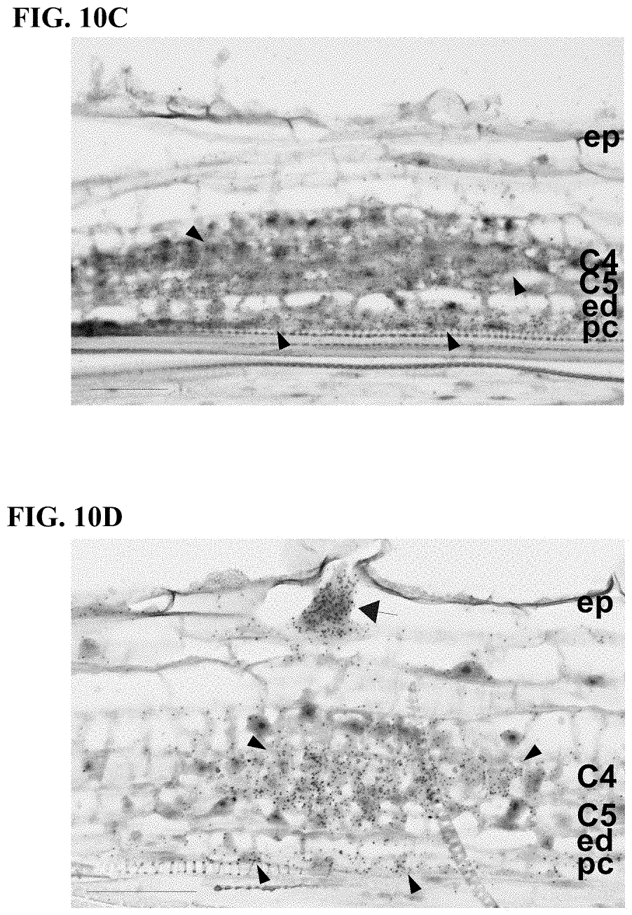

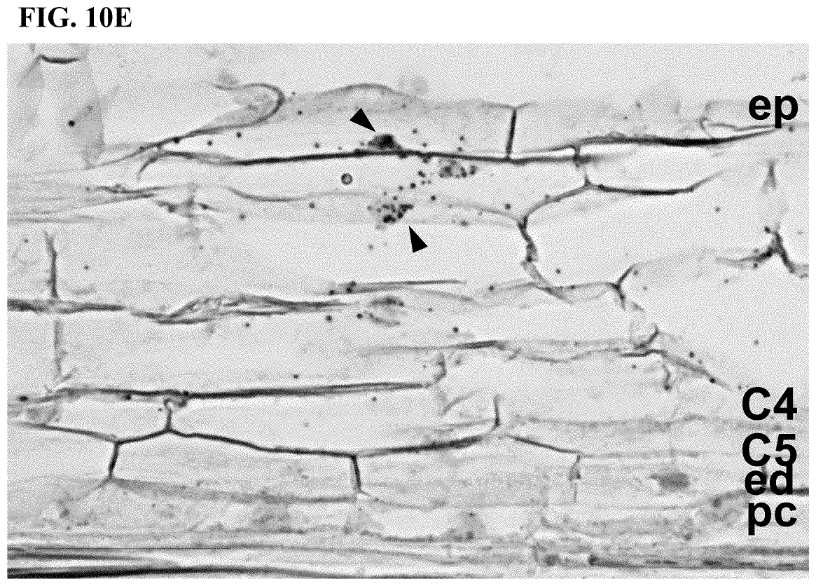

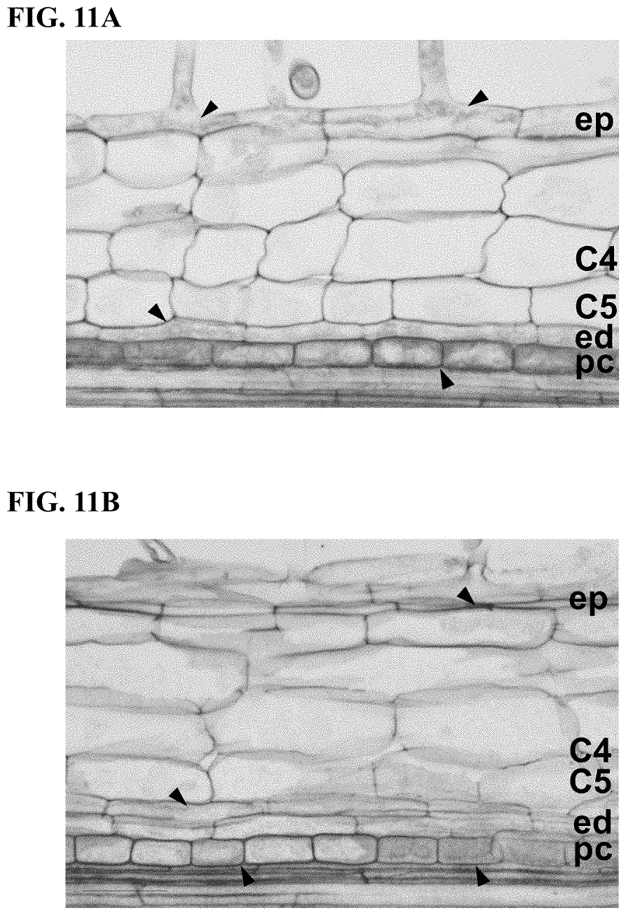

NIN expression is induced in inner root cell layers in a non-cell-autonomous way: The 2.2 kb upstream region of Mt NIN was known to be activated in the epidermis 24 hours after Nod factor application (Verne et al., Plant Cell, (2015) 27, 3410-3424). This promoter region, however, lacked the regulatory sequences shown to be required for nodule organogenesis (see above). Therefore, the expression of NIN in inner root cell layers during primordium formation was assessed via in situ hybridization. Analysis was conducted using the primordial stage in which the pericycle cells had divided and some anticlinal divisions had occurred in the inner cortical cell layers (C4 and C5) (stage used in FIGS. 10A and 10B). Analysis was also conducted on a slightly later stage when cortical cells had divided more extensively (stage used in FIGS. 10C and 10D). At both stages, the infection thread had not yet reached the primordia. As shown in FIG. 10A, at the younger stage, NIN mRNA occurred in pericycle and epidermis, but was hardly detectable in the divided cortical cells. The highest expression level occurred in the pericycle derived cells. As shown in FIG. 10C, at the older stage when cortical cells had divided more extensively, the expression level of NIN in cortex derived cells was similar to that in the pericycle. The data show that expression of NIN was first strongly induced in the pericycle, and then extended to the other inner cell layers.

NF-YA1 is a direct target of NIN (Soyano et al., PLos Genet. (2013) 9). Like NIN, NF-YA1 is expressed in the epidermis where it controls rhizobial infection (Laporte et al., J. Exp. Bot., (2014) 65, 481-494). To test whether NIN also controlled NF-YA1 expression in the primordia, RNA in situ hybridization was performed using NF-YA1 as a probe. The results demonstrated that NF-YA1 and NIN had similar expression because NF-YA1 was first induced in pericycle (FIGS. 10B and 10D). These similar expression patterns indicate that NF-YA1 may be regulated by NIN in pericycle and other nodule primordium cells. Further, the results indicate that rhizobia present in the epidermis induce NIN and NF-YA1 expression in the pericycle derived cells.

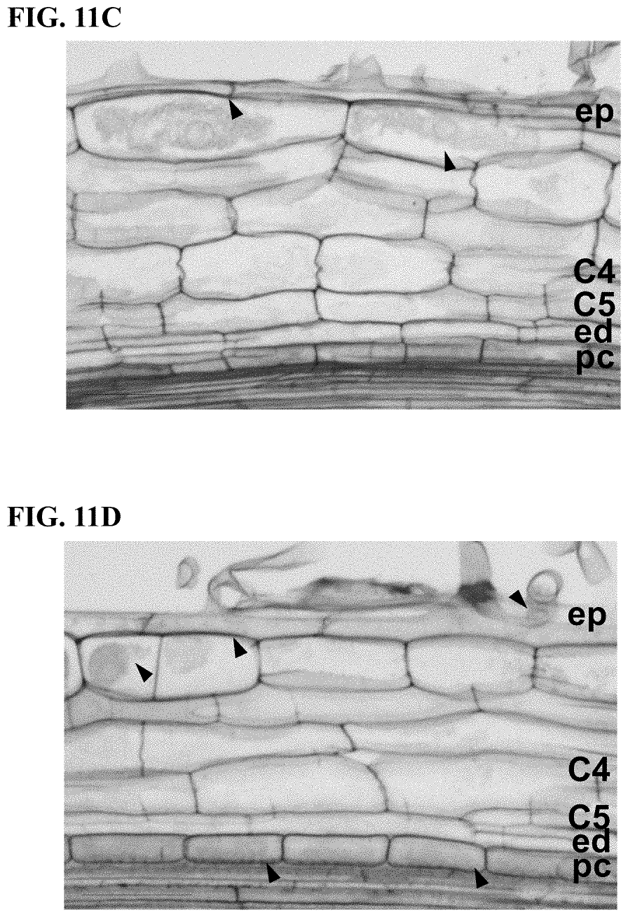

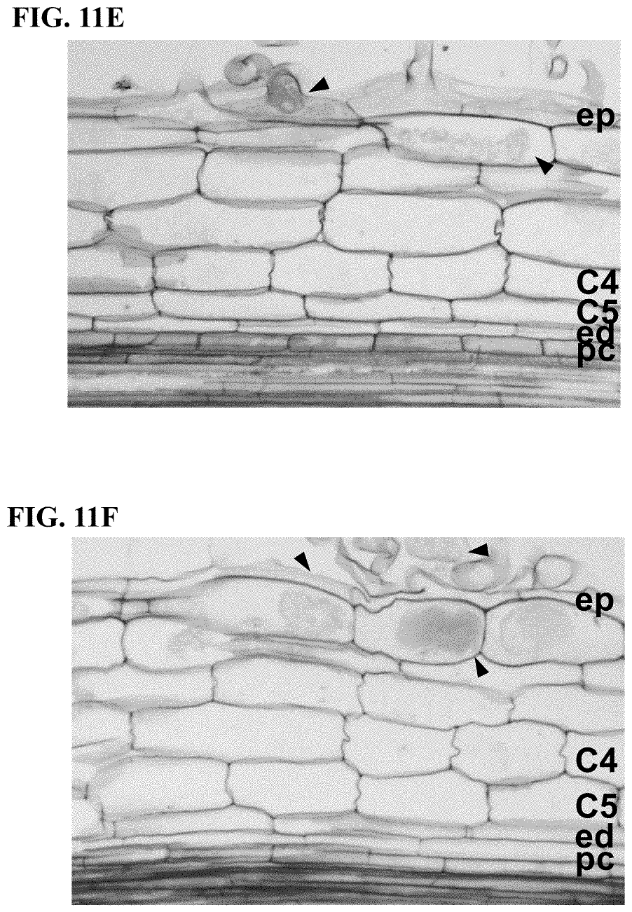

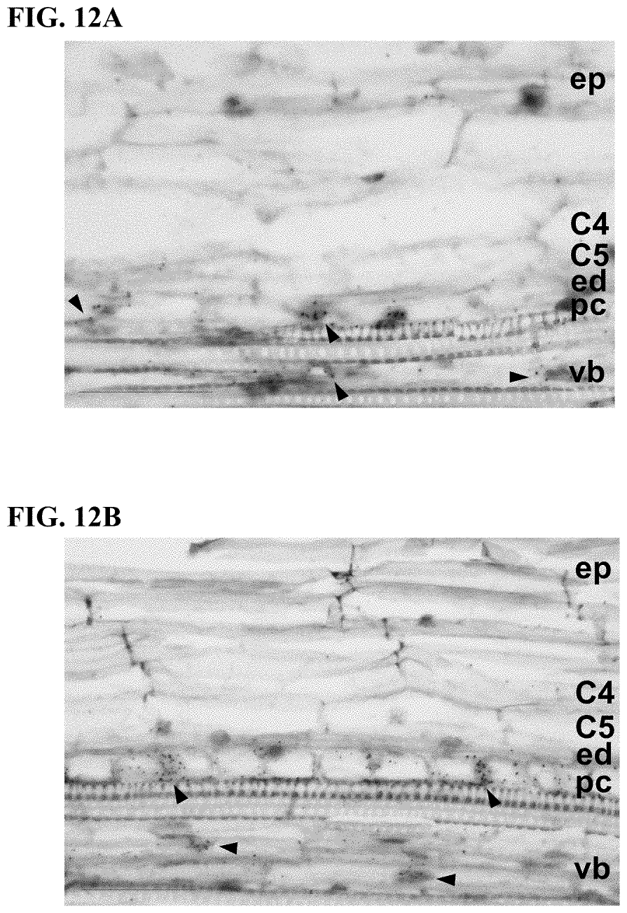

CE region is required for induction of NIN expression in pericycle: To determine whether the CE region is required for NIN expression in the inner cell layers, the expression patterns of ProNIN_{CE-5kb}:GUS and ProNIN_{5kb}:GUS were compared. Initially, both ProNIN_{CE-5kb}:GUS and ProNIN_{5kb}:GUS were introduced into A17 WT M. truncatula. Analysis was conducted on an early stage of primordium development when pericycle cells had divided and some anticlinal divisions had occurred in the inner cortical cell layers. As shown in FIGS. 11A and 11B, both constructs were expressed in the epidermis, pericycle, and endodermis while a lower signal was detected in some cortical cells. This result was unexpected given that ProNIN_{5kb}:NIN was shown to not be sufficient for primordium formation in the nin-1 background. The results indicate that expression of ProNIN_{5kb}:GUS in inner cell layers was induced by endogenous NIN that was produced in the WT background. The results further indicate that NIN expression in the inner layers is regulated by a positive feedback loop involving NIN itself, and that the essential cis-regulatory elements required for this were located in the −5 kb promoter region.

To confirm the above, ProNIN_{CE-5kb}:GUS and ProNIN_{5kb}:GUS were introduced into daphne-like by A. rhizogenes mediated transformation. In daphne-like, infection threads were formed indicating that NIN was induced in the epidermis and that the production of the mobile signal was not affected. However, nodule primordium formation was impaired, indicating there was no NIN production in the inner cell layers. Indeed, ProNIN_{5kb}:GUS transgenic roots showed GUS expression only in the epidermis and outer cortex (FIG. 11C), whereas no expression was observed in the pericycle cells. In contrast, ProNIN_{CE-5kb}:GUS transgenic roots showed GUS expression in the epidermis, outer cortex and in the pericycle (FIG. 11D). In addition, the expression of ProNIN_{CE-5kb}:GUS in the pericycle of daphne-like was weak which is consistent with the involvement of NIN in a feedback loop that positively regulates its own expression. In this case, cell division was not induced in the pericycle, due to the absence of NIN. To further demonstrate that the CE region was required for NIN expression in the pericycle, NIN expression in daphne-like primordia was examined using RNA in situ hybridization at 2 days post inoculation (dpi) with rhizobia (FIG. 10E). This showed that, unlike in WT (FIG. 10A), NIN was expressed in the epidermis and outer cortex but not in the pericycle (FIG. 10E). This result indicated that the CE region was required for NIN expression in the pericycle. Taken together, the results demonstrate that CE-controlled NIN expression in the pericycle precedes cell division in WT roots. The results indicate that the CE region is required for the initial induction of NIN expression in the pericycle.

Induction of NIN in the pericycle depends on NIN expression in the epidermis: The results above indicated that a mobile signal generated by Nod factor signaling in the epidermis induces NIN expression in the pericycle. In this case, NIN expression in the pericycle would depend on NIN induction in the epidermis. To test this, ProNIN_{CE-5kb}:GUS and ProNIN_{5kb}:GUS were introduced into nin-1 by hairy root transformation. In both cases, GUS was only present in the epidermis and outer cortex, but not in the pericycle 3 dpi (FIGS. 11E and 11F). Therefore, the data suggest that NIN was required in the epidermis to induce NIN expression in pericycle cells.

CRE1 and RR1 are expressed in the pericycle of non-inoculated roots: The occurrence of multiple B-type RR response regulatory elements in the CE region indicated that the cytokinin signaling machinery was important for NIN transcriptional activation in the pericycle. To determine whether this was in fact the case, the expression pattern of the cytokinin receptor CRE1 and its putative target the B-type RESPONSE REGULATOR RR1, which is expressed during nodule formation (Gonzalez-Rizzo et al., Plant Cell Online, (2006) 18, 2680-2693), were assessed. Using RNA in situ hybridization, it was found that CRE1 was actively transcribed in pericycle and vasculature cells of the non-inoculated roots, but not in endodermis or cortical cells (FIG. 12A). Also, mRNA of the B-type RR1 was present at the highest level in pericycle, and to a lower extent in root vasculature cells (FIG. 12B). Therefore, both CRE1 and RR1 were already expressed in the pericycle before rhizobial signaling started, indicating that initially only the pericycle layer was responsive to cytokinin.

Summary: The data presented above shows that a remote upstream regulatory region (CE) is required for the regulation of NIN expression leading to M. truncatula nodule organogenesis. The data further show that regulatory sequences for the infection process are located within a 5 kb region directly upstream of the start codon. The CE region contains several cytokinin response elements and domain 1 (D1), which contains six cytokinin response elements, is essential for nodule primordia formation. The CE region is furthermore important for the cytokinin-induced expression of NIN as the daphne-like mutant, which has an insertion disrupting CE function, has lost this ability. Nodule primordium formation starts with the induction of NIN in the pericycle and subsequently extends to the cortical cells. Further, the data demonstrate that cytokinin-linked genes CRE1 and RR1 are expressed in the pericycle. Taken together, the results indicate that cytokinin perception is involved in the induction of NIN at the start of primordium formation.

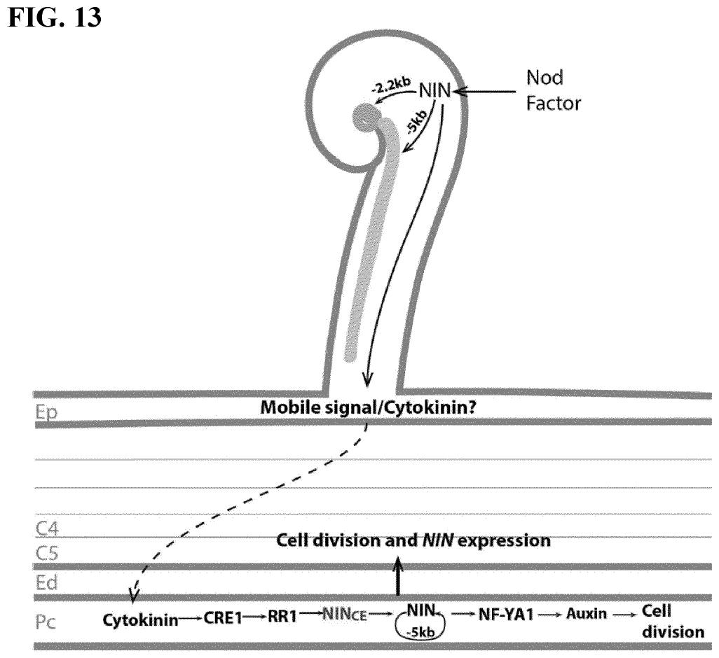

During the infection process, NIN is involved in a mechanism by which root hair growth stops when a proper curl is formed. Regulatory sequences required for this process are located within the −2.2 kb promoter region, which lacks the putative CYCLOPS binding site. Therefore, the data indicate that in addition to CYCLOPS (IPD3 in M. truncatula) another transcription factor(s) is involved in regulating NIN expression in the epidermis. Further, given that induced expression of the −2.2 kb region is not sufficient for efficient infection thread formation, the data indicate that the expression level of NIN in the epidermis remains below the threshold level required for infection thread formation, whereas the threshold level of NIN expression can be reached by induced expression of the −5 kb promoter region which includes the putative CYCLOPS binding site (FIG. 13).

A model for NIN function during nodule primordium initiation is depicted in FIG. 13. After perception of Nod factor, NIN is rapidly induced in the epidermis. The −5 kb regulatory region of the NIN promoter is sufficient for both, tight root hair curling and infection thread formation, whereas expression driven by the −2.2 kb region is only sufficient for tight root hair curling and the formation of bacterial colonies inside curl. A mobile signal is generated in the epidermis in a NIN-dependent manner and it translocates to the pericycle, where it causes cytokinin accumulation in the inner root cell layers. The CRE1 receptor in the pericycle perceives cytokinin and activates the B-type RR1, which further activates NIN expression. The induction of NIN in the pericycle requires the presence of the CE region and involves a positive feedback loop which includes NIN itself. The conclusion that the induction of NIN in the pericycle involves a positive feedback loop which includes NIN itself is supported by the observation that expression of ProNIN_{5kb}:GUS, in M. truncatula wild type background, is induced in nodule primordia although this promoter region is not sufficient to trigger primordium formation. This result is similar to a study in L. japonicus in which a promoter region of NIN that does not trigger primordium formation is sufficient to drive expression of GUS in primordia (Yoro et al., Plant Physiol., (2014) 165, 747-758; Heckmann et al., Mol. Plant-Microbe Interact, (2011) 24, 1385-1395; Kosuta et al., Plant J., (2011) 67, 929-940). NIN then directly activates NF-YA1 expression, and further stimulates cell divisions. The data indicate that NIN induction in the pericycle precedes the mitotic activation of pericycle cells. Later, the NIN-induced response in the pericycle contributes to cell division and NIN expression in the endodermis and pericycle cells.

The conclusion that nodule primordium formation requires the induction of NIN expression in inner root layers is consistent with the observation that nodule organogenesis is restored in the L. japonicus daphne mutant by NIN driven by a heterologous Arabidopsis enhancer that is active in endodermis and cortex (Yoro et al., Plany Physiol., (2014) 165, 747-758). The results above demonstrate that deletion of a region within CE containing six cytokinin response elements blocks primordium formation. This shows that cytokinin signaling in the pericycle induces NIN expression. This is further supported by the expression of the cytokinin receptor (CRE1) as well as B-type response regulator (RR1) in the pericycle before rhizobial signaling is initiated. These findings are in line with a previous study showing that a CRE1 promoter region driving GUS expression is specifically expressed in endodermis/pericycle cells opposite the protoxylem poles (Boivin et al., Plant Cell Environ., (2016) 39, 2198-2209), the sites where nodule primordia are formed (Heidstra et al., Development, (1997) 124, 1781-1787). Moreover, the importance of the CE region for cytokinin induced NIN expression is indicated by the daphne-like mutant which has lost this ability.

The CE region is conserved in the eight studied legume species. They belong to different clades of the legume Papilionoideae subfamily, representing the Genistoids, IRLC, Robinioids, Milletioids, and Dalbergioids clades. Therefore, the data indicate that regulation of NIN expression by cytokinin is conserved in this subfamily.

The results above show that after the induction of NIN in the pericycle, its expression extends to the endodermis and inner cortex. In young nodule primordia in which cortical cells have divided anticlinally (FIGS. 10A and 10B), expression of NIN as well as NF-YA1 are highest in pericycle, and it is hardly detectable in the divided cortical and endodermal cells. The results indicate that NIN induced responses in the pericycle contribute to cell division in endodermis and cortical cells (FIG. 13). At a later stage of development NIN is expressed in the dividing cortical cells (FIGS. 10C and 10D).

Cell division in nodule primordia correlates with auxin accumulation, which occurs before the first cell division (Mathesius et al., Plant J., (1998) 14, 23-34; Suzaki et al., Development, (2012) 4006, 3997-4006). The auxin accumulation (DR5 expression) depends on NIN, as it does not occur in a nin null mutant (Suzaki et al., Development, (2012) 4006, 3997-4006). Further, ectopic expression of both NIN and NF-YA1 are sufficient to induce abnormal cell division during lateral root development (Soyano et al., PLos Genet. (2013) 9), which indicates that their expression causes the local accumulation of auxin. The data presented above indicate that cytokinin signaling in the pericycle triggers NIN expression leading to the local accumulation of auxin, which subsequently triggers mitotic activity (FIG. 13). This conclusion is supported by a previous study showing that STY genes are targets of NF-YA1 (Hossain et al., Mpmi, (2016) 29, 950-964). STY genes encode transcription factors that have been shown to regulate YUCCA auxin biosynthesis genes in Arabidopsis (Eklund et al., Plant Cell, (2010) 22, 349-363; Eklund et al., Development, (2010) 137, 1275-1284; Sohlberg et al., Plant J., (2006) 47, 112-123).

1. A genetically altered plant, wherein the plant or a part thereof comprises one or more genetic alterations that increase activity of a NODULE INCEPTION (NIN) protein or a NIN-like protein (NLP protein) in response to cytokinin signaling as compared to a wild type (WT) plant without the one or more genetic alterations, and wherein the plant or the part thereof comprises a nucleic acid encoding the NIN protein or the NLP protein.

2. The genetically altered plant of claim 1, wherein the one or more genetic alterations comprise addition of one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, or twenty-four or more cytokinin response elements operably linked to the nucleic acid encoding the NIN protein or the NLP protein.

3. The genetically altered plant of claim 2, wherein at least one of the cytokinin response elements is a B-type cytokinin signaling RESPONSE REGULATOR (RR) binding site.

4. The genetically altered plant of claim 2, wherein the cytokinin response elements are within 100 nucleotides, within 90 nucleotides, within 86 nucleotides, within 80 nucleotides, within 70 nucleotides, within 60 nucleotides, within 50 nucleotides, within 40 nucleotides, within 30 nucleotides, within 25 nucleotides, within 20 nucleotides, within 19 nucleotides, within 18 nucleotides, within 17 nucleotides, within 16 nucleotides, within 15 nucleotides, within 14 nucleotides, within 13 nucleotides, within 12 nucleotides, within 11 nucleotides, within 10 nucleotides, within 9 nucleotides, within 8 nucleotides, within 7 nucleotides, within 6 nucleotides, within 5 nucleotides, within 4 nucleotides, within 3 nucleotides, within 2 nucleotides, or within 1 nucleotide of each other.

5. The genetically altered plant of claim 1, wherein the nucleic acid encoding the NIN protein or the NLP protein is operably linked to a promoter that is operably linked to the cytokinin response elements.

6. The genetically altered plant of claim 5, wherein the promoter and the cytokinin response elements are within 110,000 nucleotides, within 105,000 nucleotides, within 100,000 nucleotides, within 95,000 nucleotides, within 90,000 nucleotides, within 85,000 nucleotides, within 80,000 nucleotides, within 75,000 nucleotides, within 70,000 nucleotides, within 65,000 nucleotides, within 60,000 nucleotides, within 55,000 nucleotides, within 50,000 nucleotides, within 45,000 nucleotides, within 42,000 nucleotides, within 40,000 nucleotides, within 35,000 nucleotides, within 30,000 nucleotides, within 25,000 nucleotides, within 20,000 nucleotides, within 15,000 nucleotides, within 10,000 nucleotides, within 9,000 nucleotides, within 8,000 nucleotides, within 7,000 nucleotides, within 6,000 nucleotides, within 5,000 nucleotides, within 4,000 nucleotides, within 3,000 nucleotides, within 2,000 nucleotides, within 1,000 nucleotides, within 500 nucleotides, within 400 nucleotides, within 300 nucleotides, within 200 nucleotides, or within 100 nucleotides of each other.

7. The genetically altered plant of claim 1, wherein the nucleic acid encodes a NIN/NLP1 orthogroup protein, a NLP2-3 orthogroup protein, a NLP4 orthogroup protein, or a basal NIN/NLP orthogroup protein.

8. The genetically altered plant of claim 1, wherein the nucleic acid encoding the NIN protein or the NLP protein is endogenous.

9. The genetically altered plant of claim 1, wherein the nucleic acid encoding the NIN protein or the NLP protein is heterologous.

10. The genetically altered plant of claim 5, wherein the promoter is endogenous.

11. The genetically altered plant of claim 5, wherein the promoter is heterologous.

12. The genetically altered plant of claim 1, wherein cytokinin signaling or induction of the cytokinin signaling pathway in a root pericycle cell layer, a root endodermis cell layer (i.e., endodermal cell layer), root cortex cell layers (i.e., cortical cell layers), and/or a root epidermis cell layer (i.e., epidermal cell layer) induces nodule organogenesis.

13. The genetically altered plant of claim 1, further comprising one or more CYCLOPS response elements operably linked to the nucleic acid, wherein CYCLOPS expression in a root epidermis cell layer (i.e., epidermal cell layer) induces rhizobium infection.

14. The genetically altered plant of claim 1, wherein the genetically altered plant is a monocot, and wherein the genetically altered plant is selected from the group consisting of corn, rice, wheat, barley, sorghum, millet, oat, and rye.

15. The genetically altered plant of claim 1, wherein the genetically altered plant is selected from the group consisting of apple, pear, plum, apricot, peach, almond, walnut, cherry, strawberry, raspberry, blackberry, red currant, black currant, melon, cucumber, pumpkin, squash, grape, hemp, hops, birch, beech, jujube, cassava, poplar, chestnut, citrus, potato, tomato, sweet potato, Trema spp., and Jatropha spp.

16. A method of producing the genetically altered plant of claim 1, comprising:
a) introducing the one or more genetic alterations into a plant cell, tissue, or other explant;
b) regenerating the plant cell, tissue, or other explant into a genetically altered plantlet; and
c) growing the genetically altered plantlet into a genetically altered plant with the one or more genetic alterations that increase activity of the NIN protein or the NLP protein in response to cytokinin signaling as compared to an untransformed WT plant.

17. The method of claim 16, further comprising identifying successful introduction of the one or more genetic alterations by screening or selecting the plant cell, tissue, or other explant prior to step (b); screening or selecting plantlets between step (b) and (c); or screening or selecting plants after step (c).

18. The method of claim 16, wherein transformation is done using a transformation method selected from the group consisting of particle bombardment, Agrobacterium-mediated transformation, Rhizobium-mediated transformation, and protoplast transfection or transformation.

19. The method of claim 16, where the genetic alterations are introduced with a vector.

20. The method of claim 19, wherein the vector comprises a promoter operably linked to a nucleotide encoding a NIN or NLP protein and one or more cytokinin response elements operably linked to the promoter, and wherein the promoter and the one or more cytokinin response elements are selected from the group consisting of a NIN gene promoter comprising a 5′-upstream sequence comprising a CYCLOPS response element through a transcription start site of the NIN gene operably linked to a 3C region, the NIN gene promoter comprising a 5′-upstream sequence comprising the CYCLOPS response element through to the transcription start site of the NIN gene operably linked to a CE region, a minimal promoter operably linked to a CYCLOPS response element operably linked to a CE region, and a minimal promoter operably linked to a CYCLOPS response element operably linked to one or more cytokinin response elements.

21. The method of claim 19, wherein the vector comprises one or more gene editing components that target a nuclear genome sequence operably linked to an endogenous NIN protein or NLP protein, and wherein the nuclear genome sequence is edited by the one or more gene editing components to introduce a cis-regulatory element selected from the group consisting of one or more cytokinin response elements, a 3C region, and a CE region.

22. The method of claim 21, wherein one or more gene editing components are selected from the group consisting of a ribonucleoprotein complex that targets the nuclear genome sequence; a vector comprising a TALEN protein encoding sequence, wherein the TALEN protein targets the nuclear genome sequence; a vector comprising a ZFN protein encoding sequence, wherein the ZFN protein targets the nuclear genome sequence; an oligonucleotide donor (ODN), wherein the ODN targets the nuclear genome sequence; and a vector comprising a CRISPR/Cas enzyme encoding sequence and a targeting sequence, wherein the targeting sequence targets the nuclear genome sequence.

23. The method of claim 20, wherein the NIN gene promoter, 3C region, CE region, CYCLOPS response element, or one or more cytokinin response elements are from a nodulating legume species, and wherein the nodulating legume species is selected from the group consisting of peanut, pigeon pea, chickpea, soybean, velvet bean, bean, pea, adzuki bean, mung bean, clover, lupine, Lotus japonicus, and Medicago truncatula.
